(19) United States
(12) Patent Application Publication
(10) Pub. No.: US 2024/0299553 A1
(43) Pub. Date: Sep. 12, 2024

(54) INDOCARBOCYANINE LIPID DERIVATIVES FOR IN VIVO CARGO DELIVERY

(71) Applicants: THE REGENTS OF THE UNIVERSITY OF COLORADO, A BODY CORPORATE, DENVER, CO (US); NORTHWESTERN UNIVERSITY, EVANSTON, IL (US)

(72) Inventors: Dmitri Simberg, Centennial, CO (US); Hanmant Gaikwad, Aurora, CO (US); Irina Balyasnikova, Evanston, IL (US)

(21) Appl. No.: 18/572,447

(22) PCT Filed: Jun. 21, 2022
(86) PCT No.: PCT/US22/34302

§ 371 Date: Dec. 20, 2023

Related U.S. Application Data
(60) Provisional application No. 63/213,053, filed on Jun. 21, 2021.

(51) Int. Cl. A61K 47/54; A61K 9/127; A61K 9/51; A61K 47/69

(52) U.S. Cl. CPC A61K 47/543; A61K 9/127; A61K 9/5123; A61K 47/6911; A61K 47/6929

(57) ABSTRACT

The present disclosure relates in one aspect to constructs comprising a lipophilic membrane dye covalently linked through a linker to a cargo. In another aspect, the present disclosure relates to a method of delivering a cargo to a tumor in a subject in need thereof, the method comprising administering a construct of the disclosure to the subject. In certain embodiments, the tumor is a brain tumor.

### CROSS-REFERENCE TO RELATED APPLICATIONS

The present application claims priority under 35 U.S.C. § 119(e) to U.S. Provisional Application No. 63/213,053 filed Jun. 21, 2021, which is hereby incorporated herein by reference in its entirety.

### STATEMENT REGARDING FEDERALLY SPONSORED RESEARCH OR DEVELOPMENT

This invention was made with government support under grant numbers CA194058 and EB022040 awarded by National Institutes of Health. The government has certain rights in the invention.

### BACKGROUND OF THE INVENTION

Liposomes are the most adopted nano-sized drug carriers that comprise over 30% of all nanopharmaceuticals currently in clinical trials. Self-assembling properties, the ability to encapsulate water-soluble and lipophilic drugs, scalability, well-characterized pharmacokinetics, and biodegradability make liposomes a popular choice of therapeutic carriers. The mechanisms whereby systemically injected liposomes accumulate in tumors are not clear. In the late 80s, Maeda et al. reported the accumulation of macromolecules in tumors (termed enhanced permeability-retention (EPR) effect). The notion of pore transport as the primary mechanism for the extravasation was indirectly suggested by the presence of gaps of irregular sizes between tumor endothelial cells in mouse models. On the other hand, several authors suggested transendothelial migration of liposomes and nanoparticles, likely to be mediated by vesiculo-vacuolar organelles and caveolae. Regardless of the extravasation pathway, once liposomes and nanoparticles cross the endothelial barrier, the subsequent binding to the extracellular matrix and stroma, and uptake by immune and tumor cells are critical for efficient retention in tumors. An overwhelming majority of studies on tumoral accumulation and distribution of nanoparticles and liposomes employ fluorescence imaging and microscopy. These studies established the critical role of the nanoparticle size and geometry, physicochemical properties, tumor model, vascularization and stage.

Thus, there is a need for novel constructs that allow for delivery of cargoes, such as but not limited to therapeutic drugs and antibodies, into a tumor. The present invention addresses this unmet need.

### BRIEF DESCRIPTION OF THE DRAWINGS

The following detailed description of certain embodiments of the invention will be better understood when read in conjunction with the appended drawings. For the purpose of illustrating the invention, specific embodiments are shown in the drawings. It should be understood, however, that the invention is not limited to the precise arrangements and instrumentalities of the embodiments shown in the drawings.

FIGS. 1A-1F depict that ICLs exhibit enhanced extravasation in syngeneic solid tumors compared to FPLs, in accordance with some embodiments. FIG. 1A: EPC/DSPE-PEG2000/DiI/Cy5-DSPE liposomes were used (Table 1). FIG. 1B: Both dyes colocalized in liposomes. FIG. 1C: Confocal microscopy imaging of fresh 4T1 tumor slices shows colocalization of DiI and Cy5-DSPE in tumor vasculature, but predominant extravasation and spreading of DiI at 24 h. FIG. 1D: Quantification of fluorescence positive areas in the tumor images (n=30-60 images from 2 tumors per time point, paired t-test, repeated twice). FIG. 1E: EggPC/DSPE-PEG2000/DiI/Cy5-DSPE liposomes were injected in GL261 syngeneic intracranial glioma bearing mice. Confocal microscopy imaging of brain slices shows predominant extravasation and spreading of DiI, and an almost complete absence of Cy5-DSPE. A positive tumor (left of dotted boundary) is surrounded by normal brain tissue. FIG. 1F: Quantification of fluorescence positive areas in the microscopy images (n=40 images from 2 tumors, paired t-test).

FIG. 2 depicts a non-limiting characterization of liposomes used for the study, in accordance with some embodiments. Representative batches are shown.

FIG. 3 shows DiI/Cy5-DSPE labeled EPC/DSPE-PEG2000 liposomes that were injected in 4T1 mice, in accordance with some embodiments. Mice were injected with FITC-lectin before euthanasia to visualize blood vessels. Both dyes accumulate in blood vessels at 1 h, but DiI shows much more efficient extravasation at 24 h. Representative images (n=2 mice) are shown. The scale bar is 100 μm.

FIG. 4 depicts DiI/Cy5-DSPE labeled liposomes used in a tumor imaging dotted on a nitrocellulose membrane, in accordance with some embodiments. Dots were then imaged with a confocal microscope (left image) to calibrate the laser intensity and gain to get similar histograms in each channel (right graph).

FIG. 5 depicts DiI/Cy5-DSPE labeled EPC/DSPE-PEG2000 liposomes injected in LY2 syngeneic head and neck cancer mice, in accordance with some embodiments. Mice were injected with FITC-lectin before euthanasia to visualize blood vessels. Both dyes colocalize in blood vessels at 1 h, but DiI shows much more efficient extravasation at 24 h. Representative images (n=3 mice) are shown. The scale bar is 100 μm.

FIGS. 6A-6D depict that the type of fluorophore does not affect the enhanced extravasation of ICLs over FPLs, in accordance with some embodiments. FIG. 6A: EPC/DSPE-PEG2000/DiD/Cy3-DSPE liposomes were used (FIG. 2). FIG. 6B: Both dyes showed colocalization in liposomes. FIG. 6C: Confocal images of tumor slices. FIG. 6D: Image area quantification shows predominant spreading of DiD in tumors (n=60 images from 3 mice, paired t-test).

FIGS. 7A-7B depict DiI/Cy5-DSPE labeled HSPC/Chol/DSPE-PEG2000 liposomes injected in 4T1 mice, in accordance with some embodiments. DiI shows much more efficient extravasation (FIG. 7A) and occupies a larger tumor area (FIG. 7B) than Cy5-DSPE at 24 h. A representative image (n=3 mice) is shown. p-value<0.0001.

FIGS. 8A-8D depict that ICLs but not FPLs label tumor microenvironment, in accordance with some embodiments. FIG. 8A: EPC/DSPE-PEG2000/DiD or EPC/DSPE-PEG2000/Cy5-DSPE liposomes were prepared (FIG. 2). The probes have the same fluorescent headgroup but a different lipophilic part. FIGS. 8B-8C: 4T1 mice were injected i.v. with DiD or Cy5-DSPE liposomes. Flow cytometry of tumor single-cell suspension 4 days post-injection shows that about 17% of cells were labeled with DiD, and over 50% were F4/80+ macrophages (n=3 mice). FIG. 8D: Microscopy of tumor single-cell suspension after staining for F4/80.

FIGS. 9A-9B depict the extravasation and immune uptake of ICLs in gliomas, in accordance with some embodiments. FIG. 9A: Amino-DiI (fixable dye)-labeled liposomes were injected in GL261 or CT-2A syngeneic tumor-bearing mice or non-tumor mice. Whole-brain slices were imaged 48 h after. DiI amine shows efficient extravasation in tumors but not in the normal brain tissue. FIG. 9B: GL261 tumors were stained for myeloid marker CD11 b and general immune marker CD45.

FIGS. 10A-10E depict that both ICL and FPL are stable in liposomes and extravasate together at an early time point, in accordance with some embodiments. FIG. 10A: Incubation of DiI/Cy5-DSPE labeled liposomes in 1% Tween-20 that destroys liposomes does not significantly affect the fluorescence (repeated twice). FIG. 10B: There was less than 10% release of DiI and Cy5 fluorescence from liposomes in mouse serum (repeated twice). FIG. 10C: DiI and Cy5 in plasma after injection of DiI/Cy5-DSPE labeled liposomes show a similar elimination profile (n=3 mice). FIG. 10D: Both lipids are primarily localized in liposomes in plasma. FIG. 10E: High magnification confocal microscopy of tumor sections shows that liposomes arrive in tumors intact and extravasate blood vessels either as particles or as diffuse fluorescence. The dotted line shows the tumor endothelium lining.

FIGS. 11A-11G depict that FPLs are less stable than ICLs and are eliminated from tumors and organs in vivo, in accordance with some embodiments. FIG. 11A: Mice were injected with DiI/Cy5-DSPE labeled liposomes, and tumors (upper panel) and livers (lower panel) were excised and imaged at different time points. Representative images of merged DiI and Cy5 fluorescence are shown (n=2 mice, repeated 3 times). Liposomes were dotted on a membrane and scanned together with tumors (an image of the liposomal dot is shown in upper panel). FIG. 11B: DiI/Cy5 fluorescence ratio in tumor and liver images shows an increase over time (n=2 mice per time point). FIG. 11C: Tumors and major organs were homogenized, and the lipids were extracted with organic solvent as described in Methods. DiI (left graph) and Cy5 (right graph) show major differences in biodistribution. FIG. 11D: DiI/Cy5 fluorescence ratio in tumor and organ extracts shows an increase over time. FIG. 11E: DiI/Cy5-DSPE liposomes were spiked in liver homogenates and incubated at different times. There was a minimal increase in DiI/Cy5 fluorescence ratio over time. FIG. 11F: Thin layer chromatography analysis of fluorescence after lipid extraction from liver homogenates described in (E). Arrows point to the degradation of Cy5-DSPE. FIG. 11G: TLC analysis of liver extracts at different times post-injection of DiI/Cy5-DSPE-labeled liposomes shows the decrease in the levels and degradation of Cy5-DSPE but not DiI.

FIGS. 12A-12D depict glioma accumulation—GL261, DiD liposomal, in accordance with some embodiments. FIG. 12A: Liposomal ICL size. FIG. 12B: Extravasation and accumulations of ICLs in glioma when formulated in liposomes. FIG. 12C: Biodistribution and extravasation of liposomal ICLs in glioma. FIG. 12D: Spreading of ICLs after injection of liposomes in GL261 glioma mice.

FIG. 13 depicts extravasation—GL261, DiD liposomal, in accordance with some embodiments.

FIGS. 14A-14D depict lipid nanoparticle extravasation—GL261, DiD compared to Doxil, in accordance with some embodiments. FIG. 14A: Lipid nanoparticle (DSPE-PEG2000/DiD) size. FIG. 14B: Biodistribution in organs and glioma. FIGS. 14C-14D: Extravasation of ICLs formulated lipid nanoparticles injected in GL261 glioma.

FIG. 15 depicts a non-limiting lipid nanoparticle-invasive edge of DiD compared to Doxil, in accordance with some embodiments.

FIG. 16 depicts the accumulation of DiD liposomes and DiD lipid nanoparticles in immune cells, in accordance with some embodiments.

FIG. 17 depicts the role of complement in the uptake by immune cells, in accordance with some embodiments.

FIG. 18 depicts the invasive phenotype of GBM. Left, H&E stain of resected GBM tissue (mesenchymal molecular subtype), in accordance with some embodiments. Arrows point to infiltrating cancer cells in the surrounding brain. Dotted lines show a border of the cellular tumor. Right, Invasive phenotype of GBM12 PDX after irradiation. Nude mice bearing intracranial patient-derived GBM 12 xenografts received fractionated (2Gy for 4 days) whole-brain irradiation. H&E stain of tissue sections from control (NT) and irradiated (RT) animals' shows the invasive phenotype of tumor in irradiated mice (arrows).

FIG. 19 depicts IL13Rα2 expression (ISH) in invasive edge in GBM patients, in accordance with some embodiments. The heterogeneous expression of IL13Rα2 in GBM tissue through patients with the pronounced expression on the tumor edge.

FIG. 20 depicts the sensitivity of different GBM lines to doxorubicin and paclitaxel, in accordance with some embodiments. The IC₅₀ of doxorubicin and paclitaxel for human glioma cell lines from the Sanger/CCLE database.

FIG. 21 depicts the expression of IL13Rα2 in PDX glioma in accordance with some embodiments. Left, Flow cytometry analysis of cells harvested from PDX tissue. Staining for IL13Rα2 was with anti-IL13Rα2. A negative control is IgG1 isotype matching Ab. Center and right, staining of GBM12 in the mouse brain with mAb IL13Rα2 (Abcam) or isotype-matched IgG2a. The scale bar is 200 μm. IL13Rα2-positive GBM12 cells infiltrated into the brain (arrows).

FIGS. 22A-22C depict the synthesis of DiD and DOCy7 linked to various cargoes, in accordance with some embodiments. FIG. 22A: DiD-linked paclitaxel. FIG. 22B: DID-linked doxorubicin. FIG. 22C: DOCy7-linked antibody. IgG is shown schematically here, but scFv will be used.

FIG. 23 depicts a proposed alternative construct where both scFv and drug are conjugated to the same ICL, in accordance with some embodiments.

FIG. 24 depicts proposed Cy3-based compounds for comparison of extravasation efficiency, in accordance with some embodiments.

FIG. 25 depicts ICL-drug and ICL-antibody conjugates and combinations in accordance with some embodiments.

FIGS. 26A-26G depict that ICLs can be used as drug carriers. FIG. 26A: DiI-DyLight800-conjugate, in accordance with some embodiments. FIG. 26B: DiI-DyLight800 was formulated into PEGylated liposomes or injected as free conjugate. There was much longer circulation in PEGylated liposomes. FIGS. 26C-26D: Higher accumulation of liposomal than free conjugate in 4T1 tumors (n=3, organs of representative mice are shown).

FIG. 26E: DiI-cytarabine conjugate via a reducible linker. FIG. 26F: Release of cytarabine in 10 mM DTT. FIG. 26G: EC₅₀ of free cytarabine and the conjugate in MOLM13 leukemic cells (48 h, MTT assay; 65 nM cytarabine, 89 nM DiI-cytarabine).

FIG. 27 depicts the synthesis of indocarbocyanine lipid (ICL)-disulfidecarbamate doxorubicin, in accordance with some embodiments.

FIG. 28 depicts the synthesis of indocarbocyanine lipid (ICL)-disulfide ester linker paclitaxel, in accordance with some embodiments.

FIG. 29 depicts the mechanism of intracellular drug release using cleavable linkers, in accordance with some embodiments. The reduction of a disulfide bond by glutathione (GSH) triggers the release of the drug.

FIG. 30 depicts a) the synthesis of 2-(pyridin-2-yldisulfaneyl)ethyl 1H-1,2,4-triazole-1-carboxylate and b) The synthesis of Cytarabine disulfide linker, in accordance with some embodiments.

FIG. 31A: Synthesis of DOCy7-NH₂, in accordance with some embodiments. FIG. 31B: Synthesis of DOCy7-SS-Cytarabine, in accordance with some embodiments. FIG. 31C: Synthesis of DiI-SS-cytarabine, in accordance with some embodiments.

FIG. 32 depicts the synthesis of indocarbocyanine lipid (ICL)-doxorubicin-antibody conjugate (5), in accordance with some embodiments.

FIG. 33 depicts the synthesis of indocarbocyanine lipid (ICL)-paclitaxel-antibody conjugate (7), in accordance with some embodiments.

FIG. 34 depicts the synthesis of a DOCy7-Trichostatin A conjugate, in accordance with some embodiments.

FIG. 35 depicts the synthesis of a DOCy7-Vorinostat conjugate, in accordance with some embodiments.

FIG. 36 depicts the synthesis of a DOCy7-Dasatinib conjugate, in accordance with some embodiments.

FIG. 37 depicts the synthesis of a DOCy7-Dinaciclib conjugate, in accordance with some embodiments.

FIG. 38 demonstrates that PEGylated lipid nanoparticles (PLNs) have less accumulation than liposomes in plantar skin, in accordance with some embodiments. Upper panel: Ex vivo organ imaging 48 h post-injection of 14 nmol liposomal or PLN-formulated DiD. Lower left panel: tumor/feet ratio (after extracting DiD and normalizing to weight; n=4, t-test). Lower right panel: Confocal images of DiD accumulation in foot skin and tumor. The data suggest that formulations of ICLs can be used to avoid skin accumulation and toxicity associated with liposomes (e.g., PEGylated liposomal doxorubicin).

FIGS. 39A-39G demonstrate that indocyanine lipids can be used for drug delivery, in accordance with some embodiments. FIGS. 39A-39D: DyLight800-DiI conjugate formulated into HSPC/Chol/DSPE-PEG2000 stealth liposomes showed accumulation and penetration in freshly excised skin (imaged with NIR microscope 14 days after injection of liposomes). Many liposomes accumulate in the extravascular skin cells. Free dye clears immediately and does not show accumulation (n=3); FIGS. 39E-39G: DyLight800-DiI formulated into PEGylated, non-PEGylated EggPC liposomes or injected as free DyLight-DiI. There was a significant accumulation of DyLight in the skin of mice injected with the PEGylated formulation, 14 days post injection. D-dorsal, V-ventral, F-feet. N=3.

FIGS. 40A-40I demonstrate that Indocyanine lipids have more efficient skin accumulation than phospholipids, in accordance with some embodiments. FIG. 40A: Intravital microscopy at different time points shows extravasation by 3 h. FIG. 40B: Liposomes were formulated with Cy3-DSPE, LR-DOPE or DiI. FIG. 40C: Mouse skin was excised 4 days after injection and the microscopic areas of accumulation and extravasation were counted. DiI shows more efficient extravasation than phospholipid dyes (n=3, repeated twice). FIG. 40D-40F: circulation and skin accumulation of DiD and Cy5-DSPE liposomes (n=3 per group, 9 different skin areas pooled). FIG. 40G-40H: mixed DiI/Cy5-DSPE liposomes colocalize in skin blood vessels at 1 h, and colocalize in plasma (1000× magnification zoomed). Arrows in FIG. 40G point to DiI extravasation. FIG. 40I: 4 days later, only DiI extravasated and accumulated in skin cells (upper panel). Similar results were observed when mixed Cy3-DSPE/DiD liposomes were used (lower panel). Extravasation was observed with a confocal microscope and accumulation was imaged with a Bio-RAD gel camera equipped with Cy3 and Cy5 filters.

FIGS. 41A-41D shows certain aspects of targeting via IL13Ra2, in accordance with some embodiments. FIG. 41A: IL13Rα2 expression in invasive edge in GBM patient. FIG. 41B: DOCy7-anti-IL13Ra2-antibody. FIGS. 41C-41D: targeting of IL13Ra2-positive but not negative CT-2A glioma cells. Size bar 20 μm.

FIGS. 42A-42B show a preliminary screen of Cy3 lipid analogs in 4T1 syngeneic model in accordance with some embodiments. Lipids were formulated with DSPE-PEG2000 at 1:2 ratio to form colloidally stable PEGylated lipid nanoparticles. The representative size of DiI-C18 PLNs is 87 nm. DiI-PEG5000 was used alone without DSPE-PEG2000. 4T1 tumors were perfused and excised 48 h postinjection and imaged for Cy3 fluorescence (pseudo-colored inserts). Repeated twice.

### DETAILED DESCRIPTION OF THE DISCLOSURE

In one aspect, the present disclosure relates to a construct comprising a lipophilic membrane dye covalently linked through a linker to a cargo. In certain embodiments, the lipophilic membrane dye comprises a compound of formula (I), formula (II), formula (III), or formula (IV). In certain embodiments, the cargo is selected from the group consisting of a therapeutic drug, nucleic acid, polypeptide, enzyme, antibody, ligand, biologically active lipid, transporter substrate, dye (or chromophore), fluorophore, bioluminescent label, biosensor, contrast agent, radioisotope, hydrophilic polymer, hydrophilic copolymer, and chemiluminescent label. In certain embodiments, the cargo is a therapeutic drug. In certain embodiments, the cargo is a nucleic acid. In certain embodiments, the cargo is a polypeptide.

In certain embodiments, the cargo is an enzyme. In certain embodiments, the cargo is an antibody. In certain embodiments, the cargo is a ligand. In certain embodiments, the cargo is a biologically active lipid. In certain embodiments, the cargo is a transporter substrate. In certain embodiments, the cargo is a dye (or chromophore). In certain embodiments, the cargo is a fluorophore. In certain embodiments, the cargo is a bioluminescent label. In certain embodiments, the cargo is a biosensor. In certain embodiments, the cargo is a contrast agent. In certain embodiments, the cargo is a radioisotope. In certain embodiments, the cargo is a hydrophilic polymer. In certain embodiments, the cargo is a hydrophilic copolymer. In certain embodiments, the cargo is a chemiluminescent label. In certain embodiments, the cargo is a chemotherapeutic drug. In certain embodiments, the cargo is an antibody. In certain embodiments, the cargo is a biologically active lipid selected from a liposome or a lipid nanoparticle. In certain embodiments, the cargo is a liposome. In certain embodiments, the cargo is a lipid nanoparticle. In certain embodiments, the cargo is a liposome. In another aspect, the construct is formulated into a liposome or lipid nanoparticle.

In another aspect, the present disclosure relates to a method of delivering cargo to a tumor in a subject in need thereof, the method comprising administering a construct of the disclosure to the subject. In certain embodiments, the cargo is a chemotherapeutic drug, an antibody, or a combination thereof. In certain embodiments, the cargo is a biologically active lipid selected from a liposome or a lipid nanoparticle. In another aspect, the construct is formulated into a liposome or lipid nanoparticle. In certain embodiments, the tumor is a brain tumor. In certain embodiments, the subject has glioblastoma multiforme.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

The term “abnormal,” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics that are normal or expected for one cell or tissue type might be abnormal for a different cell or tissue type.

“About” as used herein when referring to a measurable value such as an amount, a temporal duration, and the like, is meant to encompass variations of 20% or +10%, more preferably +5%, even more preferably +1%, and still more preferably +0.1% from the specified value, as such variations are appropriate to perform the disclosed methods.

A disease or disorder is “alleviated” if the severity of a symptom of the disease or disorder, the frequency with which such a symptom is experienced by a patient, or both, is reduced.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, pulmonary and topical administration.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “pharmaceutically acceptable” refers to a material, such as a carrier or diluent, which does not abrogate the biological activity or properties of the compound, and is relatively non-toxic, i.e., the material can be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that can be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof. Pharmaceutically unacceptable salts may nonetheless possess properties such as high crystallinity, which have utility in the practice of the present invention, such as for example, utility in the process of synthesis, purification or formulation of compounds useful within the methods of the invention. Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric, galacturonic acid, glycerophosphonic acids and saccharin (e.g., saccharinate, saccharate). Suitable pharmaceutically acceptable base addition salts of compounds of the invention include, for example, metallic salts including alkali metal, alkaline earth metal, and transition metal salts such as, for example, calcium, magnesium, potassium, sodium, and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the terms “pharmaceutically effective amount” and “effective amount” and “therapeutically effective amount” refer to a nontoxic but sufficient amount of an agent to provide the desired biological result. That result can be reduction and/or alleviation of the signs, symptoms, or causes of a disease, or any other desired alteration of a biological system.

An appropriate therapeutic amount in any individual case can be determined by one of ordinary skill in the art using routine experimentation.

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound of the invention (alone or in combination with another pharmaceutical agent), to a patient, or application or administration of a therapeutic agent to an isolated tissue or cell line from a patient (e.g., for diagnosis or ex vivo applications), who has a condition contemplated herein, a symptom of a condition contemplated herein or the potential to develop a condition contemplated herein, with the purpose to cure, heal, alleviate, relieve, alter, remedy, ameliorate, improve or affect a condition contemplated herein, the symptoms of a condition contemplated herein or the potential to develop a condition contemplated herein. Such treatments can be specifically tailored or modified, based on knowledge obtained from the field of pharmacogenomics.

As used herein, the term “alkyl,” by itself or as part of another substituent means, unless otherwise stated, a straight or branched chain hydrocarbon having the number of carbon atoms designated (i.e. C₁₋₆ means one to six carbon atoms) and including straight, branched chain, or cyclic substituent groups. Examples include methyl, ethyl, propyl, isopropyl, butyl, isobutyl, tert-butyl, pentyl, neopentyl, hexyl, and cyclopropylmethyl. A non-limiting example is (C₁-C₆)alkyl, particularly ethyl, methyl, isopropyl, isobutyl, n-pentyl, n-hexyl and cyclopropylmethyl.

As used herein, the term “substituted alkyl” means alkyl as defined above, substituted by one, two or three substituents selected from the group consisting of halogen, —OH, alkoxy, —NH₂, —N(CH₃)₂, —C(═O)OH, trifluoromethyl, —C—N, —C(═O)O(C₁-C₄)alkyl, —C(═O)NH₂, —SO₂NH₂, —C(═NH)NH₂, and —NO₂, preferably containing one or two substituents selected from halogen, —OH, alkoxy, —NH₂, trifluoromethyl, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. A non-limiting example is (C₁-C₃) alkoxy, particularly ethoxy and methoxy.

As used herein, the term “halo” or “halogen” alone or as part of another substituent means, unless otherwise stated, fluorine, chlorine, bromine, or iodine atom, preferably, fluorine, chlorine, or bromine, more preferably, fluorine or chlorine.

As used herein, the term “cycloalkyl” refers to a mono cyclic or polycyclic nonaromatic radical, wherein each of the atoms forming the ring (i.e. skeletal atoms) is a carbon atom. In certain embodiments, the cycloalkyl group is saturated or partially unsaturated. In other embodiments, the cycloalkyl group is fused with an aromatic ring. Cycloalkyl groups include groups having from 3 to 10 ring atoms. Illustrative examples of cycloalkyl groups include, but are not limited to, the following moieties:

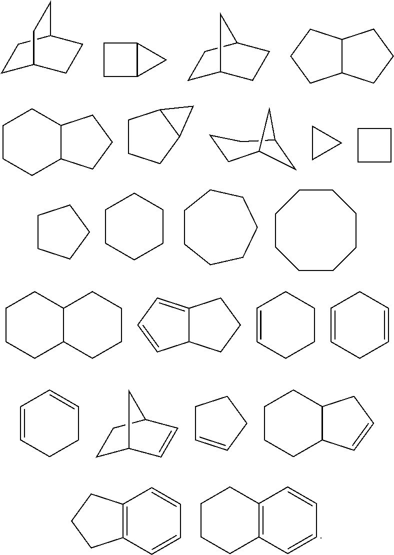

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon-carbon double bond or one carbon-carbon triple bond.

As used herein, the term “substituted” means that an atom or group of atoms has replaced hydrogen as the substituent attached to another group. The term “substituted” further refers to any level of substitution, namely mono-, di-, tri-, tetra-, or penta-substitution, where such substitution is permitted. The substituents are independently selected, and substitution can be at any chemically accessible position. In certain embodiments, the substituents vary in number between one and four. In other embodiments, the substituents vary in number between one and three. In yet other embodiments, the substituents vary in number between one and two.

As used herein, the term “optionally substituted” means that the referenced group can be substituted or unsubstituted. In certain embodiments, the referenced group is optionally substituted with zero substituents, i.e., the referenced group is unsubstituted. In other embodiments, the referenced group is optionally substituted with one or more additional group(s) individually and independently selected from the groups described herein.

In certain embodiments, the substituents are independently selected from the group consisting of oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoro alkyl, substituted or unsubstituted heteroalkyl, substituted or unsubstituted alkoxy, fluoroalkoxy, —S-alkyl, S(═O)₂alkyl, —C(═O)NH[substituted or unsubstituted alkyl], —C(═O)NH[substituted or unsubstituted phenyl], —C(═O)N[H or alkyl]₂, —OC(═O)N[substituted or unsubstituted alkyl]₂, —NHC(═O)NH[substituted or unsubstituted alkyl, or substituted or unsubstituted phenyl], —NHC(═O)alkyl, —N[substituted or unsubstituted alkyl]C(═O)[substituted or unsubstituted alkyl], —NHC(═O)[substituted or unsubstituted alkyl], —C(OH)[substituted or unsubstituted alkyl]₂, and —C(NH₂)[substituted or unsubstituted alkyl]₂. In other embodiments, by way of example, an optional substituent is selected from oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, —OCH₃, —OCH₂CH₃, —OCH(CH₃)₂, —OCF₃, —OCH₂CF₃, —S(═O)₂—CH₃, —C(═O)NH₂, —C(═O)—NHCH₃, —NHC(═O)NHCH₃, —C(═O)CH₃, and —C(═O)OH. In yet one embodiment, the substituents are independently selected from the group consisting of C₁₋₆ alkyl, —OH, C₁₋₆ alkoxy, halo, amino, acetamido, oxo and nitro. In yet other embodiments, the substituents are independently selected from the group consisting of C₁₋₆ alkyl, C₁₋₆ alkoxy, halo, acetamido, and nitro. As used herein, where a substituent is an alkyl or alkoxy group, the carbon chain can be branched, straight or cyclic.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, a description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compounds and Compositions

The compounds of the present invention can be synthesized using techniques well-known in the art of organic synthesis. The starting materials and intermediates required for the synthesis can be obtained from commercial sources or synthesized according to methods known to those skilled in the art.

The invention provides a construct comprising a lipophilic membrane dye. In certain embodiments, the lipophilic membrane dye is a derivative of DiO, DiI, DiD, or DiR. In certain embodiments, the lipophilic membrane dye is covalently linked through a linker to a cargo.

In some embodiments, the cargo is a small molecule, a nucleic acid, a peptide, a protein, and the like.

The term “small molecule,” as used herein, refers to molecules that have a molecular weight of about 1,000 or less, such as about 800 or less, about 600 or less, or about 500 or less. Examples of small molecule cargos include enzyme inhibitors, receptor ligands, allosteric modulators, and the like.

Examples of nucleic acid cargos include antisense oligonucleotides (ASOs), aptamers, miRNAs, mRNAs, plasmid DNAs, ribozymes, siRNAs, and the like.

Examples of peptide cargos include therapeutic peptides and the like.

Examples of protein cargos include antibodies, anticoagulants, blood factors, bone morphogenetic proteins, engineered protein scaffolds, enzymes, Fc fusion proteins, growth factors, hormones, interferons, interleukins, thrombolytics, and the like.

In some embodiments, the cargo is selected from the group consisting of a therapeutic drug, nucleic acid, polypeptide, protein, chemokine, aptamer, nanobody, minibody, enzyme, antibody, bispecific antibody, checkpoint inhibitor, ligand, biologically active lipid, transporter substrate, dye (or chromophore), fluorophore, bioluminescent label, biosensor, contrast agent, radioisotope, hydrophilic polymer, hydrophilic copolymer, and chemiluminescent label.

In certain embodiments, the biologically active lipid is a liposome. In certain embodiments, the biologically active lipid is a lipid nanoparticle.

In certain embodiments, the lipophilic membrane dye comprises a compound of formula (I), or formula (II), or a salt, solvate, enantiomer, diastereoisomer, tautomer, or geometric isomer thereof:

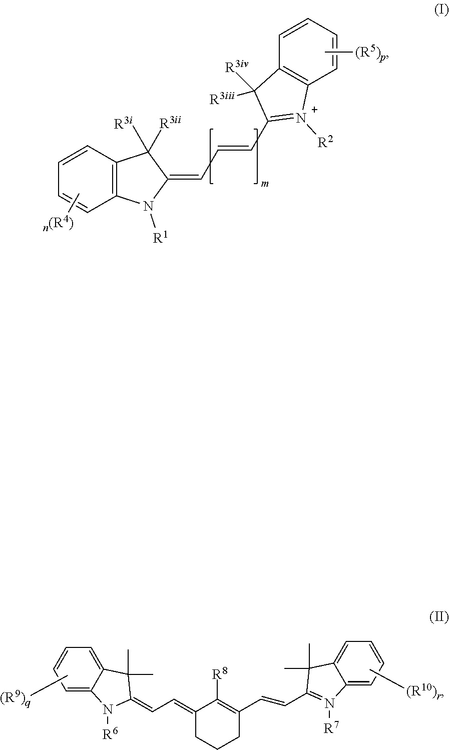

wherein:
R¹, R², R⁶, and R⁷ are each independently C₁₀-C₂₂ alkyl;
R³ⁱ, R³ⁱⁱ, R³ⁱⁱⁱ, and R^{3iv} are each independently selected from the group consisting of H and C₁-C₆ alkyl;
each occurrence of R⁴ is independently selected from the group consisting of C₁-C₆ alkyl, C₃-C₈ cycloalkyl, halo, —NO₂, —CN, —OR^{4a}, —NR^{4a}R^{4a}, —NR^{4a}—C(═O)R^{4a}, —NR^{4a}—SO₂R^{4a}, —C(═O)OR^{4a}, —C(═O)NR^{4a}R^{4a}, —SO₃H, —SR^{4a}, —S(═O)₁₋₂(C₁-C₆ alkyl), and combinations thereof, wherein each occurrence of R^{4a} is independently selected from the group consisting of H and C₁-C₆ alkyl;
each occurrence of R⁵ is independently selected from the group consisting of C₁-C₆ alkyl, C₃-C₈ cycloalkyl, halo, —NO₂, —CN, —OR^{5a}, —NR^{5a}R^{5a}, —NR^{5a}—C(═O)R^{5a}, —NR^{5a}—C(═O)OR^{5a}, —NR^{5a}—SO₂R^{5a}, —C(═O)OR^{5a}, —C(═O)NR^{5a}R^{5a}, —SO₃H, —SR^{5a}, —S(═O)₁₋₂(C₁-C₆ alkyl), and combinations thereof, wherein each occurrence of R^{5a} is independently selected from the group consisting of H and C₁-C₆ alkyl;
R⁸ is selected from the group consisting of C₁-C₆ alkyl, halo, —C(═O)OR^{8a}, —C(═O)NR^{8a}R^{8a}, —O—(C₆-C₁₂ aryl), —NR^{8a}—(C₆-C₁₂ aryl), —O—(C₄-C₁₀ heteroaryl), —NR^{8a}—(C₄-C₁₀ heteroaryl), —NR^{8a}R^{8a}, and combinations thereof, wherein R^{8a} is selected from the group consisting of H and C₁-C₆ alkyl;
each occurrence of R⁹ is independently selected from the group consisting of C₁-C₆ alkyl, C₃-C₈ cycloalkyl, halo, —NO₂, —CN, —OR^{9a}, —NR^{9a}R^{9a}, —NR^{9a}—C(═O)R^{9a}, —NR^{9a}—C(═O)OR^{9a}, —NR^{9a}—SO₂R^{9a}, —C(═O)OR^{9a}, —C(═O)NR^{9a}R^{9a}, —SO₃H, —SR^{9a}, —S(═O)₁₋₂(C₁-C₆ alkyl), and combinations thereof, wherein each occurrence of R^{9a} is independently selected from the group consisting of H and C₁-C₆ alkyl;
each occurrence of R¹⁰ is independently selected from the group consisting of C₁-C₆ alkyl, C₃-C₈ cycloalkyl, halo, —NO₂, —CN, —OR^{10a}, —NR^{10a}R^{10a}, —NR^{10a}—C(═O)R^{10a}, —NR^{10a}—C(═O)OR^{10a}, —NR^{10a}SO₂R^{10a}, —C(═O)OR^{10a}, —C(═O)NR^{10a}R^{10a}, —SO₃H, —SR^{10a}, —S(═O)₁₋₂(C₁-C₆ alkyl), and combinations thereof, wherein each occurrence of R^{10a} is independently selected from the group consisting of H and C₁-C₆ alkyl;
m is 0, 1, 2, 3, 4, 5, or 6; and
n, p, q, and r each independently is 0, 1, or 2.

In certain embodiments, R, R², R⁶, and R⁷ are each independently selected from the group consisting of C₁₂ alkyl, C₁₄ alkyl, C₁₆ alkyl, C₁₈ alkyl, C₂₀ alkyl, C₂₂ alkyl, and C₂₄ alkyl. In certain embodiments, R¹, R², R⁶, and R⁷ are each C₁₈ alkyl.

In certain embodiments, m is 1. In other embodiments, m is 2. In yet other embodiments, m is 4. In yet other embodiments m is 6.

In certain embodiments, R³ⁱ, R³ⁱⁱ, R³ⁱⁱⁱ, and R^{3iv} are each methyl.

In certain embodiments, R¹ is C₁₀-C₂₂ alkyl. In certain embodiments, R² is C₁₀-C₂₂ alkyl. In certain embodiments, R⁶ is C₁₀-C₂₂ alkyl. In certain embodiments, R⁷ is C₁₀-C₂₂ alkyl.

In certain embodiments, R³ⁱ is H. In certain embodiments, R³ⁱ is C₁-C₆ alkyl. In certain embodiments, R³ⁱⁱ is H. In certain embodiments, R³ⁱⁱ is C₁-C₆ alkyl. In certain embodiments, R³ⁱⁱⁱ is H. In certain embodiments, R³ⁱⁱⁱ is C₁-C₆ alkyl. In certain embodiments, R^{3iv} is H. In certain embodiments, R^{3iv} is C₁-C₆ alkyl.

In certain embodiments, R⁴ is C₁-C₆ alkyl. In certain embodiments, R⁴ is C₃-C₈ cycloalkyl. In certain embodiments, R⁴ is halogen. In certain embodiments, R⁴ is —NO₂. In certain embodiments, R⁴ is —CN. In certain embodiments, R⁴ is —OR^{4a}. In certain embodiments, R⁴ is —NR^{4a}R^{4a}. In certain embodiments, R⁴ is —NR^{4a}—C(═O)R^{4a}. In certain embodiments, R⁴ is —NR^{4a}—SO₂R^{4a}. In certain embodiments, R⁴ is —C(═O)OR^{4a}. In certain embodiments, R⁴ is —C(═O)NR^{4a}R^{4a}. In certain embodiments, R⁴ is —SO₃H. In certain embodiments, R⁴ is —SR^{4a}. In certain embodiments, R⁴ is —S(═O)₁₋₂(C₁-C₆ alkyl). In certain embodiments, R^{4a} is H. In certain embodiments, R^{4a} is C₁-C₆ alkyl.

In certain embodiments, R⁵ is C₁-C₆ alkyl. In certain embodiments, R⁵ is C₃-C₈ cycloalkyl. In certain embodiments, R⁵ is halogen. In certain embodiments, R⁵ is —NO₂. In certain embodiments, R⁵ is —CN. In certain embodiments, R⁵ is —OR^{5a}. In certain embodiments, R⁵ is —NR^{5a}R^{5a}. In certain embodiments, R⁵ is —NR^{5a}—C(═O)R^{5a}. In certain embodiments, R⁵ is —NR^{5a}—C(═O)OR^{5a}. In certain embodiments, R⁵ is —NR^{5a}—SO₂R^{5a}. In certain embodiments, R⁵ is —C(═O)OR^{5a}. In certain embodiments, R⁵ is —C(═O)NR^{5a}R^{5a}. In certain embodiments, R⁵ is —SO₃H. In certain embodiments, R⁵ is —SR^{5a}. In certain embodiments, R⁵ is —S(═O)₁₋₂(C₁-C₆ alkyl). In certain embodiments, R^{5a} is H. In certain embodiments, R^{5a} is C₁-C₆ alkyl.

In certain embodiments, R⁸ is C₁-C₆ alkyl. In certain embodiments, R⁸ is halogen. In certain embodiments, R⁸ is —C(═O)OR^{8a}. In certain embodiments, R⁸ is —C(═O)NR^{8a}R^{8a}. In certain embodiments, R⁸ is —O—(C₆-C₁₂ aryl). In certain embodiments, R⁸ is —NR^{8a}—(C₆-C₁₂ aryl). In certain embodiments, R⁸ is —O—(C₄-C₁₀ heteroaryl). In certain embodiments, R⁸ is —NR^{8a}—(C₄-C₁₀ heteroaryl). In certain embodiments, R⁸ is —NR^{8a}R^{8a}. In certain embodiments, R^{8a} is H. In certain embodiments, R^{8a} is C₁-C₆ alkyl.

In certain embodiments, R⁹ is C₁-C₆ alkyl. In certain embodiments, R⁹ is C₃-C₈ cycloalkyl. In certain embodiments, R⁹ is halogen. In certain embodiments, R⁹ is —NO₂. In certain embodiments, R⁹ is —CN. In certain embodiments, R⁹ is —OR^{9a}. In certain embodiments, R⁹ is —NR^{9a}R^{9a}. In certain embodiments, R⁹ is —NR^{9a}—C(═O)R^{9a}. In certain embodiments, R⁹ is —NR^{9a}—C(═O)OR^{9a}. In certain embodiments, R⁹ is —NR^{9a}—SO₂R^{9a}. In certain embodiments, R⁹ is —C(═O)OR^{9a}. In certain embodiments, R⁹ is —C(═O)NR^{9a}R^{9a} In certain embodiments, R⁹ is —SO₃H, —SR^{9a}. In certain embodiments, R⁹ is —S(═O)₁₋₂(C₁-C₆ alkyl). In certain embodiments, R^{9a} is H. In certain embodiments, R^{9a} is C₁-C₆ alkyl.

In certain embodiments, R¹⁰ is C₁-C₆ alkyl. In certain embodiments, R¹⁰ is C₃-C₈ cycloalkyl. In certain embodiments, R¹⁰ is halogen. In certain embodiments, R¹⁰ is —NO₂. In certain embodiments, R¹⁰ is —CN. In certain embodiments, R¹⁰ is —OR^{10a}. In certain embodiments, R¹⁰ is —NR^{10a}R^{10a}. In certain embodiments, R¹⁰ is —NR^{10a}—C(═O)R^{10a}. In certain embodiments, R¹⁰ is —NR^{10a}—C(═O)OR^{10a}. In certain embodiments, R¹⁰ is —NR^{10a}—SO₂R^{10a}. In certain embodiments, R¹⁰ is —C(═O)OR^{10a}. In certain embodiments, R¹⁰ is —C(═O)NR^{10a}R^{10a}In certain embodiments, R¹⁰ is —SO₃H. In certain embodiments, R¹⁰ is —SR^{10a}. In certain embodiments, R¹⁰ is —S(═O)₁₋₂(C₁-C₆ alkyl). In certain embodiments, R^{10a} is H. In certain embodiments, R^{10a} is C₁-C₆ alkyl.

In certain embodiments, m=0. In certain embodiments, m=1. In certain embodiments, m=2. In certain embodiments, m=3. In certain embodiments, m=4. In certain embodiments, m=5. In certain embodiments, m=6.

In certain embodiments, n=0. In certain embodiments, n=1. In certain embodiments, n=2.

In certain embodiments, p=0. In certain embodiments, p=1. In certain embodiments, p=2.

In certain embodiments, q=0. In certain embodiments, q=1. In certain embodiments, q=2.

In certain embodiments, r=0. In certain embodiments, r=1. In certain embodiments, r=2.

In certain embodiments, in the construct having formula (I), one or more linkers are attached directly to the phenyl ring of at least one indolinyl group. In certain embodiments, in the construct having formula (I), the one or more linkers are attached directly to the phenyl ring of at least one indolinyl group and n and/or p are 0. Therefore, In certain embodiments, the phenyl ring of the at least one indolinyl group has an open valence to which the linker forms a covalent bond. In other embodiments, in the construct having formula (I), the one or more linkers are attached to R¹, R², R⁴ and/or R⁵. Therefore, In certain embodiments, at least one of R¹, R², R⁴, and/or R⁵ has an open valence to which the linker forms a covalent bond.

In certain embodiments, in the construct having formula (I), one or more linkers are attached to R⁴ and/or R⁵. In certain embodiments, one or more of R⁴ is —(C₁-C₆ alkyl)-NR^{4a}R^{4a} and/or one or more of R⁵ is —(C₁-C₆ alkyl)-NR^{5a}R^{5a}, wherein R^{4a} and/or R^{5a} has an open valence to form a covalent bond to the linker. In certain embodiments, one or more of R⁴ is —CH₂—NR^{4a}R^{4a} and/or one or more of R⁵ is —CH₂—NR^{5a}R^{5a}, wherein R^{4a} and/or R^{5a} has an open valence to form a covalent bond to the linker. In certain embodiments, one or more of R⁴ and/or R⁵ is —CH₂—NH—, wherein one “—” represents a covalent bond to the linker.

In certain embodiments, in the construct having formula (II), the linker is attached directly to the phenyl ring of at least one indolinyl group. In certain embodiments, in the construct having formula (II), the one or more linkers are attached directly to the phenyl ring of at least one indolinyl group and q and/or r are 0. Therefore, In certain embodiments, the phenyl ring of the at least one indolinyl group has an open valence to which the linker forms a covalent bond. In other embodiments, in the construct having formula (II), one or more linkers are attached to R⁶, R⁷, R⁹ and/or R¹⁰. Therefore, In certain embodiments, at least one of R⁶, R⁷, R⁹ and/or R¹⁰ has an open valence to which the linker forms a covalent bond.

In certain embodiments, in the construct having formula (II), one or more linkers are attached to R⁸, R⁹, and/or R¹⁰. In certain embodiments, one or more of R⁹ is —(C₁-C₆ alkyl)-NR^{9a}R^{9a} and/or one or more of R¹⁰ is —(C₁-C₆ alkyl)-NR^{10a}R^{10a}, wherein R^{10a} and/or R^{10a} has an open valence to form a covalent bond to the linker. In certain embodiments, one or more of R⁹ is —CH₂—NR^{9a}R^{9a} and/or one or more of R¹⁰ is —CH₂—NR^{10a}R^{10a}, wherein R^{9a} and/or R^{10a} has an open valence to form a covalent bond to the linker. In certain embodiments, one or more of R⁹ and/or R¹⁰ is —CH₂—NH—, wherein one “—” represents a covalent bond to the linker. In certain embodiments, R⁸ is —O—(C₆-C₁₂ aryl)-(C₁-C₆ alkyl)-NR^{8a}R^{8a}, —NR^{8a}—(C₆-C₁₂ aryl)-(C₁-C₆ alkyl)-NR^{8a}R^{8a}, —O—(C₄-C₁₀ heteroaryl)-(C₁-C₆ alkyl)-NR^{8a}R^{8a}, or —NR^{8a}—(C₄-C₁₀ heteroaryl)-(C₁-C₆ alkyl)-NR^{8a}R^{8a}, wherein one R^{8a} of NR^{8a}R^{8a} has an open valence to form a covalent bond to the linker. In certain embodiments, R⁸ is O—(C₆-C₁₂ aryl)-(C₁-C₆ alkyl)-NR^{8a}R^{8a} or —NR^{8a}—(C₆-C₁₂ aryl)-(C₁-C₆ alkyl)-NR^{8a}R^{8a}, wherein C₆-C₁₂ aryl is phenyl, C₁-C₆ alkyl is C₁ alkyl (CH₂), and one R^{8a} of NR^{8a}R^{8a} has an open valence to form a covalent bond to the linker. In certain embodiments, R⁸ is

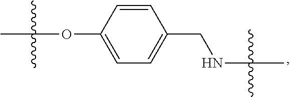 [SMILES: *NCc1ccc(O*)cc1]

wherein one [symbol] indicates a bond to the cyclohexene ring of formula (II) and one [symbol] indicates a bond to the linker.

The linker can be any organic linker known to a person of skill in the art. In certain embodiments, the linker comprises a bond that can be cleaved in an intracellular environment. In certain embodiments, the cleavable bond is a disulfide, a carbamate, an ester, an amide, a thioester, a disulfide carbamate, or a hydrazone.

In certain embodiments, the linker is a disulfide linker. In certain embodiments, the disulfide linker comprises *—C(═O)—(C₁-C₆ alkyl)-S—S—(C₁-C₆ alkyl)-C(═O)—, wherein * indicates the bond between the linker and the compound of formula (I) or (II). In certain embodiments, the C₁-C₆ alkyl is a linear alkyl. In other embodiments, the C₁-C₆ alkyl is a branched alkyl. In certain embodiments, each instance of C₁-C₆ alkyl is —CH₂CH₂—. In certain embodiments, the linker is *—C(═O)—(CH₂)₂—S—S—(CH₂)₂—C(═O)—, wherein * is a bond to an R⁴, R⁵, R⁹, or R¹⁰ of —CH₂—NH and the terminal “—” is a bond formed to an —OH on the cargo. In other embodiments, the disulfide linker comprises *—C(═O)—(C₁-C₆ alkyl)-S—S—(C₁-C₆ alkyl)-OC(═O)—, wherein * indicates the bond between the linker and the compound of formula (I) or (II). In certain embodiments, the C₁-C₆ alkyl is a linear alkyl. In other embodiments, the C₁-C₆ alkyl is a branched alkyl. In certain embodiments, each instance of C₁-C₆ alkyl is —CH₂CH₂—. In certain embodiments, the linker is *—C(═O)—(CH₂)₂—S—S—(CH₂)₂—OC(═O)—, wherein * is a bond to an R⁴, R⁵, R⁹, or R¹⁰ of —CH₂—NH and the terminal “—” is a bond formed to an —NH₂ on the cargo. In other embodiments, the disulfide linker comprises

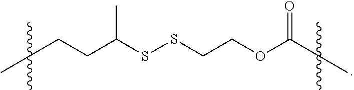 [SMILES: *CCC(C)SSCCOC(*)=O]

In certain embodiments, the linker is —CH₂—C(═O)S—, —CH₂—C(═O)O—, or —CH₂—C(═O)NH—. In other embodiments, the linker is —O(C═O)NH—. In yet other embodiments, the linker is —(CH₂)₂—S—S—(CH₂)₂—C(═O)NH—. In yet other embodiments, the linker is —(CH₂)₂—S—S—(CH₂)₂—OC(═O)NH—. In yet other embodiments, the linker is —CH₂—C(═O)NH—N═.

In certain embodiments, the linker is *—CH₂—C(═O)—S—, *—CH₂—C(═O)—O—, or *—CH₂—C(═O)—NH—, *—O(C═O)NH—, *—(CH₂)₂—S—S—(CH₂)₂—C(═O)NH—, *—(CH₂)₂—S—S—(CH₂)₂—OC(═O)NH—, or *—CH₂—C(═O)NH—N═, wherein * indicates the bond to R⁴, R⁵, R⁸, R⁹, or R¹⁰ and the other terminal “—” indicates the bond to the cargo. In other embodiments, the linker is *—CH₂—C(═O)—S—, *—CH₂—C(═O)—O—, or *—CH₂—C(═O)—NH—, *—O(C═O)NH—, *—(CH₂)₂—S—S—(CH₂)₂—C(═O)NH—, *—(CH₂)₂—S—S—(CH₂)₂—OC(═O)NH—, or *—CH₂—C(═O)NH—N═, wherein * indicates the bond to a phenyl ring of at least one indolinyl group of formula (I) or formula (II) and the other terminal “—” indicates the bond to the cargo.

In certain embodiments, the linker comprises a C₁-C₂₀ hydrocarbon chain. In other embodiments, the linker is —C(═O)—. In other embodiments, the linker is —C(═O)—(C₁-C₆ alkyl)-C(═O)—. In other embodiments, the linker comprises 1-20 amino acids. In certain embodiments, the linker comprises one or more amino acids and a terminal p-aminocarbamate (PABC). In certain embodiments, the linker is valine-citrulline-PABC. In other embodiments, the linker is glutamic acid-valine-citrulline-PABC. In yet other embodiments, the linker is N-(carbobenzyloxy)-L-phenylalanine-lysine-PABC. In yet other embodiments, the linker comprises at least one —OCH₂CH₂— group. In yet other embodiments, the linker comprises from 1 to about 5,000 —OCH₂CH₂— groups. In yet other embodiments, the linker is conjugated to the cargo through a 3-thio-succinimido group.

In certain embodiments, the linker comprises formula (A), (B) or (C):

*—(CH₂)ₘ₁—X₁—(CH₂—CH₂—X₂)ₘ₂—(CH₂)ₘ₃—C(X₃)— (A)

*—(CH₂)ₘ₁—O—(CH₂—CH₂—O)ₘ₂—(CH₂)ₘ₃—C(O)— (B)

*—(CHR′)ₘ₁—O—(CHR′—CHR′—O)ₘ₂—(CHR′)ₘ₃—C(O)— (C)

wherein:
* indicates the bond between the linker and the compound of formula (I) or (II); each m1, m2, and m3 is independently an integer ranging from 0-5000;
each X₁, X₂, and X₃ is independently absent (a bond), O, or N—R′;
each R′ is independently selected from the group consisting of hydrogen, optionally substituted C₁-C₆ alkyl, optionally substituted C₃-C₈ cycloalkyl, and optionally substituted C₃-C₈ cycloheteroalkyl.

In certain embodiments, the linker is formed via a click chemistry between trans-cyclooctene (TCO) and methyl tetrazine (MTz). In certain embodiments, MTz is appended on a —(CH₂)NH— R⁴, R⁵, R⁹, or R¹⁰ via one or more —OCH₂CH₂— groups. In certain embodiments, the combination of R⁴, R⁵, R⁹, or R¹⁰ with the appended MTz has the following structure:

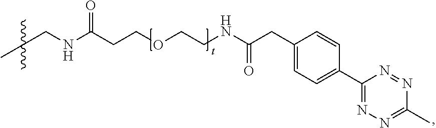

wherein t is an integer between 1 and about 5,000. In certain embodiments, the TCO is appended to the cargo via one or more —OCH₂CH₂— groups. In certain embodiments, the TCO appended to one or more —OCH₂CH₂— groups has the following structure:

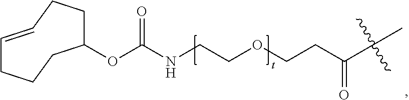

wherein t is an integer between 1 and about 5,000 and [symbol] indicates the bond to the cargo. In certain embodiments, t is 4.

In certain embodiments, the linker formed by the click chemistry between TCO and MTz has the following structure:

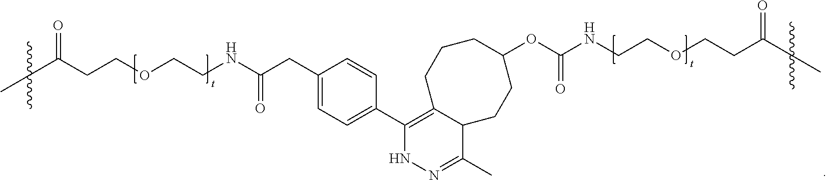

In certain embodiments, the linker forms a bond to more than one cargo. In certain embodiments, the linker that forms a bond to more than one cargo comprises both a disulfide linker described elsewhere herein which bonds to one cargo and a linker formed by click chemistry between TCO and MTz described elsewhere herein which bonds to a second cargo.

In certain embodiments, the linker that bonds to one cargo and the linker that bonds to a second cargo are connected to each other via the following structure:

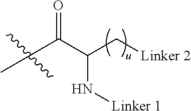

to form one linker that binds to two cargoes, wherein u is an integer from 1 to 10. In certain embodiments, “Linker 1” is —C(═O)—(C₁-C₆ alkyl)-S—S—(C₁-C₆ alkyl)-C(═O)—. In certain embodiments, each C₁-C₆ alkyl of “Linker 1” is —CH₂CH₂—. In certain embodiments, one of the terminal ketones of “Linker 1” is bound to a therapeutic drug cargo. In certain embodiments, one of the terminal ketones of “Linker 1” is bound to —OH on the therapeutic drug cargo. In other embodiments, “Linker 1” is —C(═O)—(C₁-C₆ alkyl)-S—S—(C₁-C₆ alkyl)-O—C(═O)—. In certain embodiments, each C₁-C₆ alkyl of “Linker 1” is —CH₂CH₂—. In certain embodiments, the terminal ester of “Linker 1” is bound to a therapeutic drug cargo. In certain embodiments, the terminal ester of “Linker 1” is bound to —NH₂ on the therapeutic drug cargo. In certain embodiments, “Linker 2” is

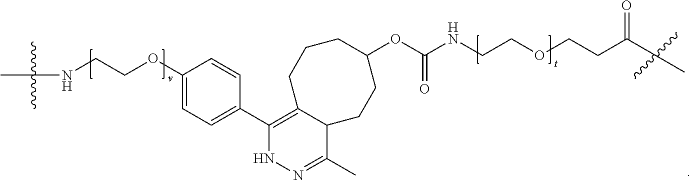

wherein v is an integer from 1 to 5,000. In certain embodiments, each v is 4. In certain embodiments, the terminal amine of “Linker 2” is bound to one of the CH₂ groups of “u” in the above structure and the terminal ketone is bound to one of the cargoes. In certain embodiments, the terminal ketone is bound to an antibody cargo. In certain embodiments, u is 4.

In certain embodiments, the compound of formula (I) comprises the compound of formula (III), or a salt, solvate, enantiomer, diastereoisomer, tautomer, or geometric isomer thereof:

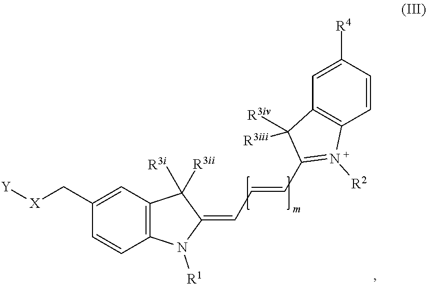

wherein:
R¹, R², R³ⁱ, R³ⁱⁱ, R³ⁱⁱⁱ, R^{3iv}, and m are defined elsewhere herein;
R⁴ is H or —SO₃H;
X is selected from —NR—, —NR—C(═O)—, —NR—C(═O)O—, —C(═O)—,

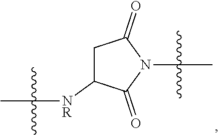

### —NR—S(═O)₂—, —O—, —OC(═O)—, —S—, —S(C═O)—, and —CRR—;

Y comprises the linker conjugated to the cargo; and each R is independently selected from H and C₁-C₆ alkyl.

In certain embodiments, R⁴ is H. In certain embodiments, R⁴ is —SO₃H.

In certain embodiments, X is —NR—. In certain embodiments, X is —NR—C(═O)—. In certain embodiments, X is —NR—C(═O)O—. In certain embodiments, X is —C(═O)—. In certain embodiments, X is

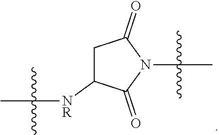

In certain embodiments, X is —NR—S(═O)₂—. In certain embodiments, X is —O—. In certain embodiments, X is —OC(═O)—. In certain embodiments, X is —O—. In certain embodiments, X is —S(C═O)—. In certain embodiments, X is —CRR—.

In certain embodiments, R is H. In certain embodiments, R is C₁-C₆ alkyl.

In certain embodiments, X is —NR— wherein R is H. In certain embodiments, Y is a linker described elsewhere herein.

In certain embodiments, the compound of formula (II) comprises the compound of formula (IV), or a salt, solvate, enantiomer, diastereoisomer, tautomer, or geometric isomer thereof:

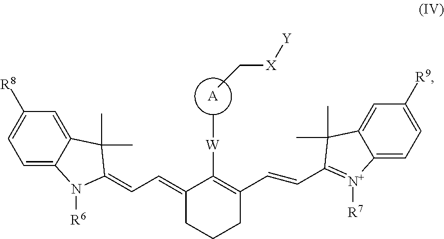

wherein:
R⁶ and R⁷ are defined elsewhere herein;
R⁸ and R⁹ are each independently H or —SO₃H;
W is —O— or —NR—;
[symbol] is C₆-C₁₂ aryl or C₄-C₁₀ heteroaryl;
X is present and selected from —NR—, —NR—C(═O)—, —NR—C(═O)O—, —C(═O)—,

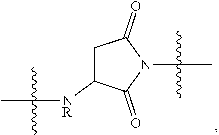

—NR—S(═O)₂—, —O—, —OC(═O)—, —S—, —S(C═O)—, and —CRR—, or X is absent;
Y comprises the linker conjugated to the cargo; and each R is independently selected from H and C₁-C₆ alkyl.

In certain embodiments, R⁸ is H. In certain embodiments, R⁹ is H. In certain embodiments, R⁸ is —SO₃H. In certain embodiments, R⁹ is —SO₃H.

In certain embodiments, W is —O—. In certain embodiments, W is —NR—.

In certain embodiments, [symbol] is C₆-C₁₂ aryl. In certain embodiments, [symbol] is C₄-C₁₀ heteroaryl.

In certain embodiments, X is present. In certain embodiments, X is —NR—. In certain embodiments, X is —NR—C(═O)—. In certain embodiments, X is —NR—C(═O)O—. In certain embodiments, X is —C(═O)—. In certain embodiments, X is

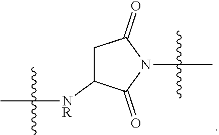

In certain embodiments, X is —NR—S(═O)₂—. In certain embodiments, X is —O—. In certain embodiments, X is —OC(═O)—. In certain embodiments, X is —S—. In certain embodiments, X is —S(C═O)—.

In certain embodiments, X is —CRR—. In certain embodiments, X is absent.

In certain embodiments, R is H. In certain embodiments, R is C₁-C₆ alkyl.

In certain embodiments, W is —O—. In certain embodiments, is phenyl. In certain embodiments, X is present and is —NR—. In certain embodiments, X is —NH—. In other embodiments, X is absent and Y is directly bonded to CH₂ of formula (IV). In certain embodiments,

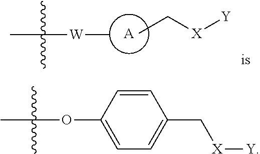

In certain embodiments, Y is a linker described elsewhere herein.

In certain embodiments, the cargo is a therapeutic drug. In certain embodiments, the drug is a chemotherapeutic drug. Exemplary chemotherapeutic drugs include, but are not limited to, doxorubicin, auristatin, paclitaxel, cytarabine, trichostatin A, vorinostat, dasatinib, dinaciclib, camptothecin, and/or STING agonists. In other embodiments, the drug is an immunostimulant drug. In certain embodiments, the immunostimulant drug is a drug that enhances immune infiltrations, reprograms macrophages, and/or depletes specific populations of immune cells. In certain embodiments, the cargo is a nucleic acid, such as but not limited to a siRNA, mRNA, miRNA, DNA, oligodeoxynucleotide, and so forth.

In certain embodiments, the cargo is a polypeptide.

In certain embodiments, the cargo is a ligand, such as but not limited to folate, RGD, VEGF, Sialyl-Lewis molecule, and so forth.

In certain embodiments, the cargo is an enzyme, such as but not limited to superoxide dismutase (SOD), asparaginase, protease, catalase, and so forth.

In certain embodiments, the cargo comprises tetrazine, such as but not limited to 4-methyl-tetrazine (MTz).

In certain embodiments, the cargo is an antibody. In certain embodiments, the antibody targets and binds the high-affinity IL13 receptor IL13Rα2. In certain embodiments, the antibody is an IL13Rα2 scFv that binds to IL13Rα2 but not IL13Rα1. In certain embodiments, the antibody is a whole antibody, an scFv, a nanobody, or a minibody.

In certain embodiments, the cargo is a bioactive or biologically active lipid. In certain embodiments, the biologically active lipid is a liposome. In other embodiments, the biologically active lipid is a lipid nanoparticle.

In certain embodiments, the cargo is a transporter substrate. In certain embodiments, the transporter substrate is transferrin.

In certain embodiments, the cargo is a hydrophilic polymer. In certain embodiments, the hydrophilic polymer comprises at least one selected from the group consisting of polyethylene glycol, polyvinylpyrrolidone, polyethylenimine, polymethacrylate, and polyvinyl alcohol.

In certain embodiments, the cargo is a hydrophilic copolymer. In certain embodiments, the hydrophilic copolymer comprises at least one polymer selected from the group consisting of polyethylene glycol, polyvinylpyrrolidone, polyethylenimine, polymethacrylate, and polyvinyl alcohol, or any copolymer thereof.

In certain embodiments, the cargo is a dye or chromophore, such as but not limited to a near-infrared dye (Cy7, IRDye800, Cy5.5, and so forth).

In certain embodiments, the cargo is a fluorophore.

In certain embodiments, the cargo is a bioluminescent label.

In certain embodiments, the cargo is a chemiluminescent label.

In certain embodiments, the cargo is a biosensor, such as but not limited to an enzyme sensor, pH sensor, hypoxia sensor, metabolite sensor, and so forth.

In certain embodiments, the cargo is a contrast agent, such as a chelator that can complex gadolinium, iron oxide, perfluorocarbon bubble, iodine, and so forth.

In certain embodiments, the cargo is a radioisotope, such as but not limited to chelated Actinium 225, chelated Tc99m, chelated Lutecium-177, chelated Cu-64, F-18, and so forth.

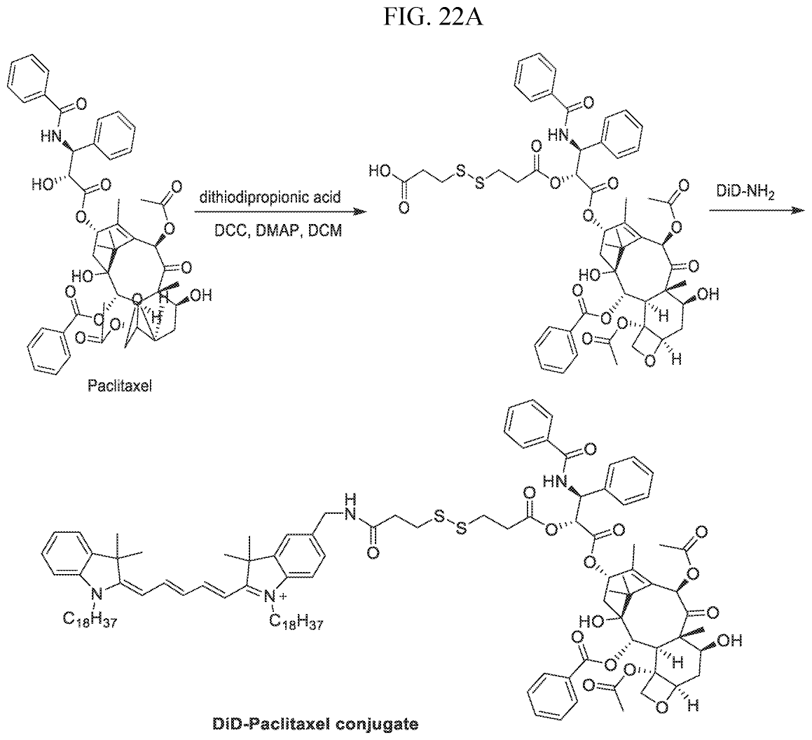

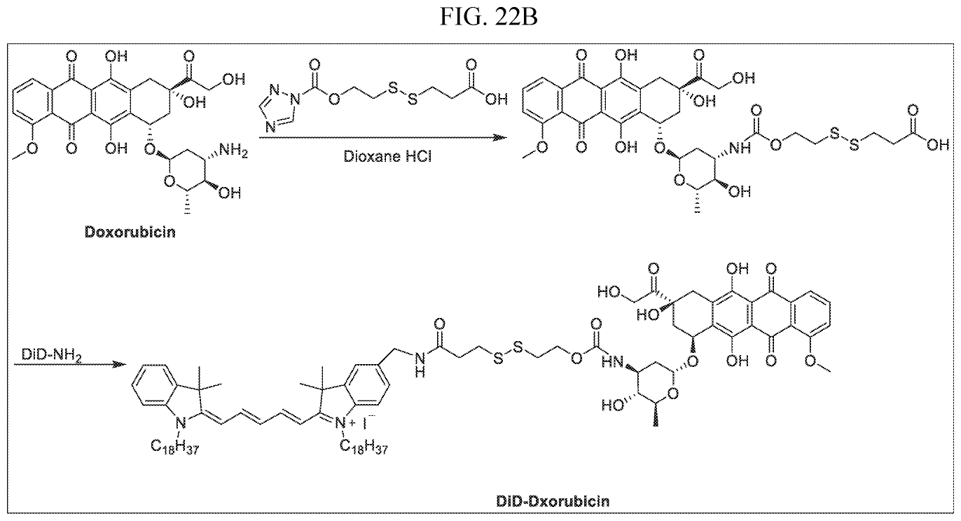

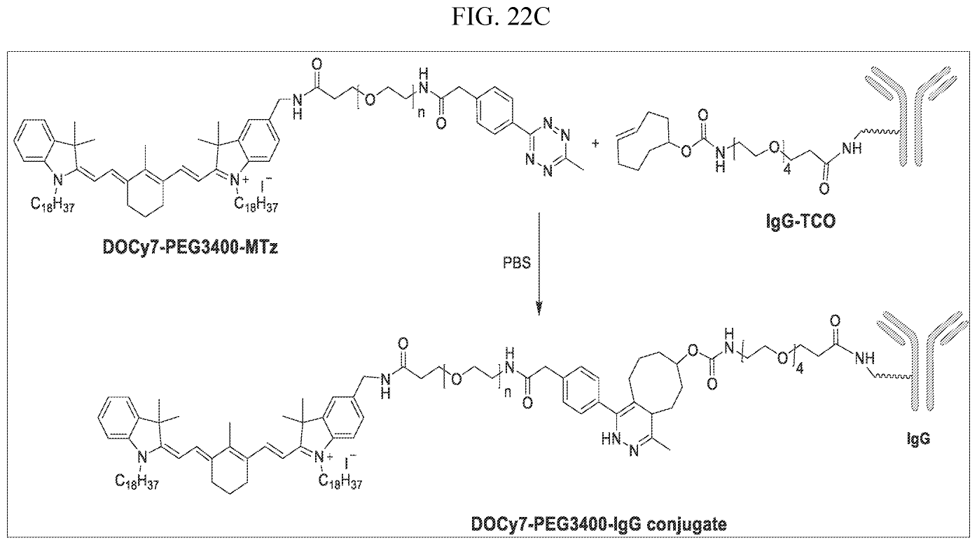

In some embodiments, the construct is a construct depicted in any one of FIG. 22A-22C, 23, 27, 28, 31B, 31C, or 32-37.

In another aspect, the present disclosure relates to a composition comprising one or more constructs of the disclosure. In certain embodiments, the composition comprises a construct of the disclosure formulated into a liposome or a lipid nanoparticle. In certain embodiments, the liposome or lipid nanoparticle is PEGylated. In certain embodiments, the liposome or lipid nanoparticle has a diameter of between about 0.1 nm and about 10,000 nm, about 0.1 nm and about 8,000 nm, about 0.1 nm and about 6,000 nm, about 0.1 nm and about 4,000 nm, about 1 nm and about 4,000 nm, or about 5 nm and about 2,000 nm. In certain embodiments, the composition comprises at least one pharmaceutically acceptable carrier. Exemplary pharmaceutical carriers are described elsewhere herein. In certain embodiments, the composition is formulated for administration by a route selected from the group consisting of oral, parenteral, transdermal, transmucosal, intravesical, intrapulmonary, intraduodenal, intragastrical, intrathecal, subcutaneous, intramuscular, intradermal, intra-arterial, intravenous, intrabronchial, inhalation, and topical.

The compounds of the disclosure can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound of the invention, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compounds of the invention may exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compounds of the invention are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Ci, ¹⁸F, ¹²³I, ¹²⁵I, ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000, 2001), and Green & Wuts, Protective Groups in Organic Synthesis 3rd Ed., (Wiley 1999) (all of which are incorporated by reference for such disclosure). General methods for the preparation of compounds as described herein are modified by the use of appropriate reagents and conditions for the introduction of the various moieties found in the formula as provided herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal. In certain embodiments, protective groups are removed by acid, base, reducing conditions (such as, for example, hydrogenolysis), and/or oxidative conditions.

### Methods

In another aspect, the present disclosure relates to a method of delivering a cargo to a tumor in a subject in need thereof, the method comprising administering a construct of the disclosure to the subject.

The subject can be any mammal who has a tumor. In certain embodiments, the subject is a human subject. In certain embodiments, the subject has a tumor associated with bone cancer, blood cancer (such as leukemia, lymphoma and myeloma), brain cancer, breast cancer, colorectal cancer, liver cancer, lung cancer, head and neck cancer, ovary cancer, pancreatic cancer, prostate cancer, skin cancer, stomach cancer, or a combination thereof. In certain embodiments, the subject has a tumor associated with brain cancer. Exemplary types of brain cancer include, but are not limited to, astrocytomas, meningiomas, oligodendrogliomas, ependymomas, mixed gliomas, mixed glial and neuronal tumors, and primitive neuroectodermal tumors. In certain embodiments, the subject has a tumor associated with glioblastoma multiforme (GBM).

The construct can be any construct described elsewhere herein. In certain embodiments, the construct comprises a lipophilic membrane dye of formula (I), formula (II), formula (III), or formula (IV). Although not wishing to be limited by theory, it is believed that the disclosed lipophilic membrane dyes and constructs comprising such dyes demonstrate increased extravasation in tumors compared to other dyes, such as fluorescent phospholipids. In certain embodiments, the lipophilic membrane dye is covalently linked to one or more linkers wherein the one or more linkers are each covalently linked to one or more cargoes. Exemplary linkers and cargoes are described elsewhere herein. In certain embodiments, the linker is cleavable linker. In certain embodiments, the cleavable linker is cleaved in an intracellular environment. In certain embodiments, the cargo comprises one or more chemotherapeutic drugs. Exemplary chemotherapeutic drugs are described elsewhere herein. In other embodiments, the cargo comprises an antibody. Exemplary antibodies are described elsewhere herein. In certain embodiments, the antibody is an IL13Rα2 scFv. Although not wishing to be limited by theory, it is believed that IL13Rα2 is overexpressed at the invasive edge of GBM tumors. Therefore, in certain embodiments, the delivery of a construct comprising an IL13Rα2 scFv cargo can be used to treat GBM in a subject in need thereof. In yet other embodiments, the cargo comprises a biologically active lipid. Exemplary biologically active lipids are described elsewhere herein. In some embodiments, the construct is a component of a composition. In certain embodiments, the composition further comprises one or more pharmaceutically acceptable carriers. Exemplary pharmaceutically acceptable carriers are described elsewhere herein. In certain embodiments, the construct comprises a lipophilic membrane dye of formula (I), formula (II), formula (III), or formula (IV) covalently bonded via a linker to a liposome or lipid nanoparticle cargo. In other embodiments, the construct comprises a lipophilic membrane dye of formula (I), formula (II), formula (III), or formula (IV) covalently bonded via a linker to a cargo wherein the construct is formulated into a liposome or a lipid nanoparticle. In some embodiments, the liposome or lipid nanoparticle is PEGylated. Although not wishing to be limited by theory, it is believed that the presence of a liposome or lipid nanoparticle improves the penetration of the construct or a composition comprising the construct through the blood-brain barrier (BBB) and/or blood tumor barrier (BTB).

In certain embodiments, the construct is co-formulated with other lipids or additives to make liposomes or lipid nanoparticles or other lipid assemblies. These include various mole % of neutral lipids (e.g. DSPC, DOPC, EggPC, cholesterol), different mole ratios of PEGylated lipids (e.g. 750 Da to 20,000 kDa), and negatively and positively charged lipids (e.g. DSPG, phosphatidic acid and DOTAP). Although not wishing to be limited by theory, it is believed that the inclusion of helper lipids may be necessary to impart long-circulating properties and in vivo stability and affect penetration. In certain embodiments, the liposome, lipid nanoparticle, or other lipid assembly is made by mixing one or more lipids in a common solvent and drying. The lipid film is then hydrated in a suitable solvent and sonicated/vortexed. Particles of different sizes are prepared by standard extrusion through polycarbonate membranes (e.g. 400 nm, 200 nm, or 50 nm).

The construct can be administered to the subject using any method known to a person of skill in the art. Exemplary administration methods include, but are not limited to, oral, parenteral, transdermal, transmucosal, intravesical, intrapulmonary, intraduodenal, intragastrical, intrathecal, subcutaneous, intramuscular, intradermal, intra-arterial, intravenous, intrabronchial, inhalation, systemic, and topical. In certain embodiments, the construct is injected into a subject. In certain embodiments, the cargo has a higher circulating half-life in the subject when compared to a cargo that is not part of a construct disclosed herein. In certain embodiments, the subject is further administered at least one additional therapeutically effective agent.

In yet another aspect, the present disclosure relates to a method of delivering a cargo to a skin pathology in a subject in need thereof, the method comprising administering a construct of the disclosure to the subject, wherein the construct is constructed to have improved skin targeting efficiencies by, for example, including the lipophilic membrane dye described elsewhere herein with or without additional PEGylation in the construct.

In yet another aspect, the present disclosure relates to a method of delivering a cargo in a subject while avoiding significant delivery to the skin of the subject, the method comprising administering a construct of the disclosure to the subject, wherein the construct is constructed to have reduced skin targeting efficiencies by, for example, excluding the lipophilic membrane dye described elsewhere herein and including PEGylation in the construct.

The subject can be any mammal which has skin pathology. In certain embodiments, the subject is a human subject.

The construct can be any construct described elsewhere herein. In certain embodiments, the construct comprises a lipophilic membrane dye of formula (I), formula (II), formula (III), or formula (IV). Although not wishing to be limited by theory, it is believed that the disclosed lipophilic membrane dyes and constructs comprising such dyes demonstrate increased extravasation into skin when compared to other dyes, such as fluorescent phospholipids. In certain embodiments, the lipophilic membrane dye is covalently linked to one or more linkers, wherein the one or more linkers are each covalently linked to one or more cargoes. Exemplary linkers and cargoes are described elsewhere herein. In certain embodiments, the linker is a cleavable linker. In certain embodiments, the cleavable linker is cleaved in an intracellular environment. In certain embodiments, the cargo comprises one or more therapeutic drugs. The therapeutic drug can be any therapeutic drug to treat a skin pathology in the subject. In yet other embodiments, the cargo comprises a biologically active lipid. Exemplary biologically active lipids are described elsewhere herein.

In some embodiments, the construct is a component of a composition. In certain embodiments, the composition further comprises one or more pharmaceutically acceptable carriers. Exemplary pharmaceutically acceptable carriers are described elsewhere herein.

In certain embodiments, the construct comprises a lipophilic membrane dye of formula (I), formula (II), formula (III), or formula (IV) covalently bonded via a linker to a liposome or lipid nanoparticle cargo. In other embodiments, the construct comprises a lipophilic membrane dye of formula (I), formula (II), formula (III), or formula (IV) covalently bonded via a linker to a cargo wherein the construct is formulated into a liposome or a lipid nanoparticle. In some embodiments, the liposome or lipid nanoparticle is PEGylated. Although not wishing to be limited by theory, it is believed that the presence of a liposome or lipid nanoparticle improves the spread of the construct into the skin tissue of the subject and/or the length of time that the construct remains in the tissue.

The construct can be administered to the subject using any method known to a person of skill in the art. Exemplary administration methods include, but are not limited to, oral, parenteral, transdermal, transmucosal, intravesical, intrapulmonary, intraduodenal, intragastrical, intrathecal, subcutaneous, intramuscular, intradermal, intra-arterial, intravenous, intrabronchial, inhalation, and topical. In certain embodiments, the construct is administered to a subject with a skin pathology via topical, subcutaneous, intradermal, or system administration. In certain embodiments, the cargo has a higher circulating half-life in the subject when compared to a cargo that is not part of a construct disclosed herein. In certain embodiments, the subject is further administered at least one additional therapeutically effective agent.

### Administration Dosage Formulations

The regimen of administration may affect what constitutes an effective amount. The therapeutic formulations can be administered to the subject either prior to or after the onset of diseases or disorders contemplated herein. Further, several divided dosages, as well as staggered dosages, can be administered daily or sequentially, or the dose can be continuously infused or can be a bolus injection. Further, the dosages of the therapeutic formulations can be proportionally increased or decreased, as indicated by the exigencies of the therapeutic or prophylactic situation.

Administration of the compositions of the present invention to a patient, preferably a mammal, more preferably a human, can be carried out using known procedures, at dosages and for periods of time effective to treat, ameliorate, or prevent diseases or disorders contemplated herein. An effective amount of the therapeutic compound necessary to achieve a therapeutic effect may vary according to factors such as the state of the disease or disorder in the patient; the age, sex, and weight of the patient; and the ability of the therapeutic compound to treat, ameliorate, or prevent diseases or disorders contemplated herein. Dosage regimens can be adjusted to provide the optimum therapeutic response. For example, several divided doses can be administered daily or the dose can be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention can be varied so as to obtain an amount of the active ingredient that is effective in achieving the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle.

The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of heart failure in a patient.

In certain embodiments, the compositions of the invention are formulated using one or more pharmaceutically acceptable excipients or carriers. In certain embodiments, the pharmaceutical compositions of the invention comprise a therapeutically effective amount of a compound of the invention and a pharmaceutically acceptable carrier.

The carrier can be a solvent or dispersion medium containing, for example, water, ethanol, polyol (for example, glycerol, propylene glycol, and liquid polyethylene glycol, and the like), suitable mixtures thereof, and vegetable oils.

In certain embodiments, the compositions of the invention are administered to the patient in dosages that range from one to five times per day or more. In other embodiments, the compositions of the invention are administered to the patient in a range of dosages that include but are not limited to, once every day, every two, days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physical taking all other factors about the patient into account.

Compounds of the invention for administration can be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In certain embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of diseases or disorders contemplated herein.

Formulations can be employed in admixtures with conventional excipients, i.e., pharmaceutically acceptable organic or inorganic carrier substances suitable for oral, parenteral, nasal, intravenous, subcutaneous, enteral, or any other suitable mode of administration known to the art. The pharmaceutical preparations can be sterilized and if desired, mixed with auxiliary agents, e.g., lubricants, preservatives, stabilizers, wetting agents, emulsifiers, salts for influencing osmotic pressure buffers, coloring, flavoring and/or aromatic substances, and the like. They may also be combined where desired with other active agents, e.g., other analgesic agents.

Routes of administration of any of the compositions of the invention include oral, nasal, rectal, intravaginal, parenteral, buccal, sublingual or topical. The compounds for use in the invention can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are tablets, dragees, liquids, drops, suppositories, or capsules, caplets and gelcaps. The compositions intended for oral use can be prepared according to any method known in the art and such compositions may contain one or more agents selected from the group consisting of inert, non-toxic pharmaceutically excipients that are suitable for the manufacture of tablets. Such excipients include, for example, an inert diluent such as lactose; granulating and disintegrating agents such as cornstarch; binding agents such as starch; and lubricating agents such as magnesium stearate. The tablets can be uncoated or they can be coated by known techniques for elegance or to delay the release of the active ingredients. Formulations for oral use may also be presented as hard gelatin capsules wherein the active ingredient is mixed with an inert diluent.

### Parenteral Administration

For parenteral administration, the compounds of the invention can be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents can be used.

### Additional Administration Forms

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patent Applications Nos. 20030147952; 20030104062; 20030104053; 20030044466; 20030039688; and 20020051820. Additional dosage forms of this invention also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of the present invention can be, but are not limited to, short-term, rapid-offset, as well as controlled, for example, sustained release, delayed-release and pulsatile release formulations.

The term sustained release is used in its conventional sense to refer to a drug formulation that provides for a gradual release of a drug over an extended period of time, and that may, although not necessarily, result in substantially constant blood levels of a drug over an extended time period. The period of time can be as long as a month or more and should be a release that is longer than the same amount of agent administered in bolus form.

For sustained release, the compounds can be formulated with a suitable polymer or hydrophobic material, which provides sustained release properties to the compounds. As such, the compounds for use in the method of the invention can be administered in the form of microparticles, for example, by injection or in the form of wafers or discs by implantation.

In certain embodiments of the invention, the compounds of the invention are administered to a patient, alone or in combination with another pharmaceutical agent, using a sustained release formulation.

The term delayed-release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for a release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present invention depends on the age, sex and weight of the patient, the current medical condition of the patient and the progression of heart failure in the patient being treated. The skilled artisan is able to determine appropriate dosages depending on these and other factors.

A suitable dose of a compound of the present invention can be in the range of from about 0.01 mg to about 5,000 mg per day, such as from about 0.1 mg to about 1,000 mg, for example, from about 1 mg to about 500 mg, such as about 5 mg to about 250 mg per day. The dose can be administered in a single dosage or in multiple dosages, for example, from 1 to 4 or more times per day. When multiple dosages are used, the amount of each dosage can be the same or different. For example, a dose of 1 mg per day can be administered as two 0.5 mg doses, with about a 12-hour interval between doses.

It is understood that the amount of compound dosed per day can be administered, in non-limiting examples, every day, every other day, every 2 days, every 3 days, every 4 days, or every 5 days. For example, with every other day administration, a 5 mg per day dose can be initiated on Monday with a first subsequent 5 mg per day dose administered on Wednesday, a second subsequent 5 mg per day dose administered on Friday, and so on.

In the case wherein the patient's status does improve, upon the doctor's discretion, the administration of the inhibitor of the invention is optionally given continuously; alternatively, the dose of the drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the viral load, to a level at which the improved disease is retained. In certain embodiments, patients require intermittent treatment on a long-term basis upon any recurrence of symptoms and/or infection.

The compounds for use in the method of the invention can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form can be for a single daily dose or one of the multiple daily doses (e.g., about 1 to 4 or more times per day). When multiple daily doses are used, the unit dosage form can be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosages for use in humans. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood that modifications in reaction conditions, including but not limited to reaction times, reaction size/volume, and experimental reagents, such as solvents, catalysts, pressures, atmospheric conditions, e.g., a nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### Examples

The invention is now described with reference to the following Examples. These Examples are provided for the purpose of illustration only and the invention should in no way be construed as being limited to these Examples but rather should be construed to encompass any and all variations which become evident as a result of the teaching provided herein.

### Example 1: Liposomal Extravasation and Accumulation in Tumors as Studied by Fluorescence Microscopy and Imaging Depend on the Fluorescent Label

Indocarbocyanine lipids (ICLs) are very popular lipophilic fluorescent dyes for labeling of liposomes and nanoparticles as well as for long-term tracking of cells. ICLs are stable in membranes and exhibit minimal transfer and exchange in serum, possibly due to the highly lipophilic nature and mild cationic charge. Headgroup-modified fluorescent phospholipids (FPLs), for example, lissamine rhodamine phosphatidylethanolamine, is another type of popular lipophilic dyes used for liposomal labeling. Despite the popularity of these two classes of dyes, their tumoral accumulation and distribution have not been compared side by side.

Herein, liposomes were prepared that were dual-labeled with dioctadecyl ICLs (C18-DiI or C18-DiD) and distearoyl FPLs (Cy5-DSPE or Cy3-DSPE). These fluorophores have long C18 alkyl or acyl chains to enable stable retention in liposomes, and similar cyanine fluorophore headgroups (DiI is the dioctadecyl derivative of Cy3, and DiD is the dioctadecyl derivative of Cy5). To allow side-by-side comparison and to exclude the effect of size and composition of liposomes, both ICL and FPL were incorporated in the same liposome. The extravasation and accumulation of the dyes were compared in biologically relevant, syngeneic immunocompetent tumor models by ex vivo confocal microscopy and ex vivo imaging. It was found that ICLs and FPLs accumulated in tumor blood vessels and extravasated to the same extent at early time points, but there was a much better migration and retention of ICLs at late time points, which also included labeling of the immune cells. Unlike ICLs, the gradual disappearance of FPLs from tumors and other organs was observed. The data point to the instability of FPLs in tissues. These data suggest that trafficking and stability of fluorescent dyes is an important factor that could affect the conclusion about the magnitude of the EPR effect and long-term accumulation of liposomes in tumors and other organs.

### Materials and Methods

### Materials

DiD (1,1′-Dioctadecyl-3,3,3′,3′-Tetramethylindotricarbocyanine, 4-chlorobenzenesulfonate salt) and DiI (1,1′-Dioctadecyl-3,3,3′,3′-Tetramethylindotricarbocyanine Perchlorate) were from Biotium (Hayward, CA, USA) and were stored as stock in ethanol. Whatman Nucleopore Track-Etch Membranes, bovine serum albumin, and the chemicals for FPL synthesis were from Sigma-Aldrich (St. Louis, MO, USA). Nitrocellulose membrane (0.45 μm) and PVDF membrane was from Bio-Rad (Hercules, CA, USA). Hydrogenated soy phosphatidylcholine, egg phosphatidylethanolamine, distearoyl phosphatidylethanolamine (DSPE), cholesterol, DSPE-PEG2000 were from Avanti Polar Lipids (Alabaster, AL, USA) and were kept as chloroform stocks at −20° C. Anti-mouse CD11b, anti-mouse CD45 and anti-mouse F4/80 were from BioLegend (San Diego, CA, USA). Nuclear staining reagent Hoechst 33342 trihydrochloride trihydrate was purchased from Life Technologies (Carlsbad, CA, USA). Fetal bovine calf serum, RPMI 1640, and DMEM growth medium supplemented with L-glutamine were from Corning Inc. (New York, NY, USA). FITC-labeled tomato lectin (FL-1171-1) was from Vector Laboratories (Burlingame, CA, USA).

### Synthesis of Cy5-DSPE and Cy3-DSPE

A mixture of DSPE-NH₂ (8 mg, 0.01 mmol, 1 eq.) Cy5 NHS or Cy3-NHS (11.4 mg 0.015 mmol, 1.5 eq.) and DIEA (5.7 μl, 0.03 mmol, 3 eq.) was stirred in chloroform: methanol (9:1) at 50° C. for 2 hours. The solvent was then evaporated under reduced pressure, and the resulting dark blue residue was purified by preparatory HPLC (C18 column) and eluted with 70% to 80% methanol/water (0.1% TFA), to obtain the product. The product was characterized by MALDI TOF mass spectrometry and was found to be 1212.77 Da for Cy5-DSPE and 1185.74 Da for Cy3-DSPE.

### Liposome Preparation

Lipids at the following molar ratios: HSPC/Chol/DSPE-PEG2000 (57/38/5) or EPC/DSPE-PEG2000 (95:5) with addition of 0.2% ICL and/or 0.2% FPL were mixed and dried under a nitrogen stream. The dry lipid cake was resuspended in PBS for a total lipid concentration of 20 mM, then incubated at 60° C. for 30 minutes. The solution was then vortexed for 2 minutes and bath sonicated. Liposomes were extruded by a syringe extruder (Avestin, Ottawa, Canada) through Whatman Nucleopore Track-Etch Membranes (200 nm pore size, 15 times). HSPC-based liposomes were extruded at 60° C., EPC-based liposomes were extruded at room temperature. Size and zeta potential were measured at room temperature in the presence of 1% phosphate-buffered saline using Zetasizer Nano (Malvern, UK). Liposomes were stored at 4° C. at a final concentration of 10 mM (total lipid) for a maximum period of 8 weeks before use.

### Animal Experiments

4T1 cell line was purchased from the ATCC and verified using a sequencing core. GL261 cells were obtained from the National Cancer Institute (NCI), and CT2A cells were a gift from Dr. Tom Seyfried (Boston College). LY2 cells were from Dr. Sana Karam, University of Colorado Anschutz Medical campus. Cells were grown at 37° C. in RPMI 1640 medium containing 10% heat-inactivated fetal bovine serum, 10 mM HEPES, 100 U/ml penicillin, and 100 ng/ml streptomycin (all from Corning Inc. New York, NY, USA). BALB/c mice were bred in-house. Mice of 8-10 weeks of age (females) were implanted with 0.5×10⁶ cells into the mammary fat pad (4T1) or subcutaneously in the jaw region (LY2) and used for experiments 1 week later when tumors reached ˜200-300 mm³. For brain tumors, C57BL/6 mice (6-8 weeks old) were implanted with a total 1×10⁵ GL261 or CT-2A cells per animal using an established protocol. To determine the distribution of liposomes in tumors and organs, liposomes were injected with 50 μl of 10 mM total lipid intravenously. Blood was collected via periorbital plexus using heparin glass capillaries, and the plasma was separated by centrifugation at 500 g for 5 min. In some experiments, mice were preinjected before dissection with 50 μl of FITC-tomato lectin (1 mg/ml) and 50 μl Hoechst 33324 (2 mg/ml) to visualize the vasculature and the nuclei. Mice were sacrificed with carbon dioxide, followed by cardiac perfusion with PBS through the left ventricle.

### Microscopy

Nikon Eclipse AR1HD inverted confocal microscope with 405 nm, 488 nm, 561 nm, and 640 nm excitation lasers and corresponding emission filters was used. For high magnification imaging, liposomes were diluted 1:200 in PBS, mixed with glycerol at 1:1 ratio and 2 μl were placed on slide. For liposomes in plasma, the samples were diluted 1:20 and mixed with glycerol. The preparations in glycerol were spread under a coverslip and imaged under Apo60 or Apo100 Oil objective at 2048×2048 resolution. For tumor imaging, tumors or whole brains were frozen on dry ice and sliced with a blade into 1-2 mm thick slices. The slices were placed on a glass slide and imaged using Plan Apo 10 objective. Channels' voltage and laser intensity were adjusted using the dual labeled liposomes placed on a slide and imaged under the same magnification. These parameters were calibrated before each imaging session using the batch of liposomes used in that experiment. For quantification, multiple random image areas from different slices of the tumor (both periphery and center) were acquired at 512×512 resolution. Images were quantified with Fiji using customized macros. Briefly, 8-bit gray image stacks were thresholded for fluorescence in the Cy3 and Cy5 channels and the percentages of binary (positive) areas were calculated using the “Measure” function. All the data were plotted with Prism version 8.3.0 (GraphPad, San Diego, CA).

### Flow Cytometry and Immunostaining

A single-cell suspension of tumor tissue was prepared for flow cytometry analysis, as previously described. A single cell isolates were pre-blocked with Ultra-LEAF purified anti-mouse CD16/32 antibody (BioLegend, San Diego, CA) in PBS supplemented with 2% FBS 10 min at 4° C. before antibody staining. 4T1 single-cell suspension was stained with anti-F4/80-AF488. After incubation on ice for 20 minutes and washing, cells were analyzed for staining using the Guava EasyCyte HT flow cytometer (Merck KGaA). Cells were resuspended at ˜0.5 million/ml, and 20,000 events were detected. Dead cells and fragments were excluded with FSC/SSC dot plot, and the percentage of Cy5+ cells was analyzed with FlowJo software Version 10.6 (FlowJo, LLC). For immunostaining, mice with GL261 tumors were injected with DiI-amine liposomes and perfused 48 h post-injection. Brains were snap-frozen in OCT in liquid nitrogen and sectioned with a cryostat into 5-8 μm sections. The sections were fixed with 4% formalin on a slide, blocked with 10% goat serum, and stained with anti-CD11b and CD45 antibodies and corresponding secondary antibodies.

### Dye Stability in Serum and Liver Homogenates

Liposomes were incubated at 2 mM in 80% mouse serum, PBS or 1% Tween/PBS for different times at 37° C. Following incubation, the samples were dotted on a nitrocellulose membrane and scanned at Cy3 and Cy5 wavelengths. Alternatively, to measure the release in serum, the samples were diluted after incubation 10-fold in PBS and ultracentrifuged at 80,000 rpm for 30 min, 4° C. using TLA-100.3 rotor of Beckman Optima ultracentrifuge. The supernatant in serum, liposomes in whole serum, and liposomes in PBS were dotted and scanned at Cy3 and Cy5 wavelengths with a Bio-Rad camera imager using Cy3 and Cy5 filters. The mean fluorescence of the dots was determined and used to calculate the percentage of release in serum. To measure the effect of tissue on DiI/Cy5 fluorescence ratio, livers from BALB/c mice were homogenized for 5 min in PBS (1:2 weight: volume ratio) using BioSpec Mini BeadBeater-16 tissue homogenizer with 1-2 zirconium beads added per tube. Liposomes were added at 1:10 ratio to the homogenate and incubated at 37° C. for different times. The homogenate was further diluted 10-fold, dotted on a nitrocellulose membrane and scanned with Bio-Rad gel imager at Cy3 and Cy5 channels as described above.

### Organ and Plasma Imaging

Tumors and livers were placed in a 24-well plate and scanned as described above. To match the intensity of the channels, the same liposomes used for injection were diluted (1:10), dotted on a 0.45 μm nitrocellulose membrane, and scanned together with the samples. Mean fluorescence was determined from 8-bit TIFF images using Fiji software by drawing a region of interest around the tumors and using the “Measure” function to determine mean gray values. Such measurement is independent of the organ cross-section area. The mean gray values of non-injected tumors were subtracted from the measurements. For plasma fluorescence profile, plasma collected at different time points was serially diluted, dotted on a nitrocellulose membrane, scanned as above, and the mean fluorescence of dots was plotted versus time with Prism.

### Organ Extractions and Thin-Layer Chromatography (TLC)

For extraction using a modified Bligh-Dyer method, 50-100 mg tissue was homogenized as described above, 10 parts of chloroform/methanol (2:1) were added to the homogenate (considering that tissue weight is 1 part), and the samples were mixed at 1400 rpm for 2 h at room temperature on Eppendorf Thermomixer. The tubes were centrifuged at 500 g for 10 min. The organic phase (bottom, approximately 80% of the added amount) was carefully collected, dotted on a PVDF membrane and scanned for DiI and Cy5 fluorescence as described above. Different dilutions of liposomes (standard curve) were scanned together with the samples. The percentage of the injected DiI and Cy5 in the extract was calculated from the standard curve and divided by tissue weight. The fluorescence of control non-injected organs was subtracted from that of injected organs. For TLC analysis, the mobile phase consisted of chloroform: methanol (9:1) with 1% trifluoroacetic acid and the samples were run on TLC Silica Gel 60 F254 plates (EMD Millipore).

### Results

### Liposomal ICLs and FPLs Show Similar Accumulation in Tumors at Early Time Points, but Drastic Differences at Late Time Points

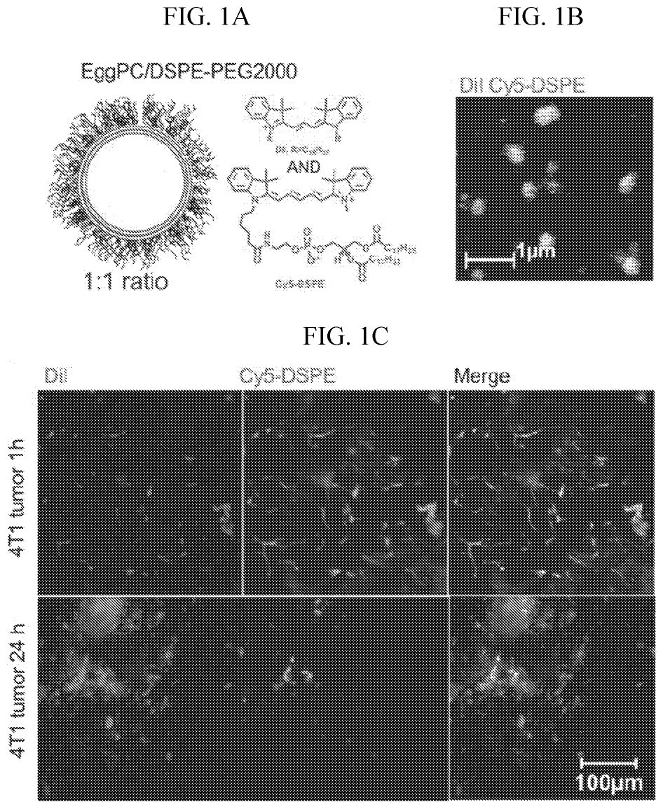

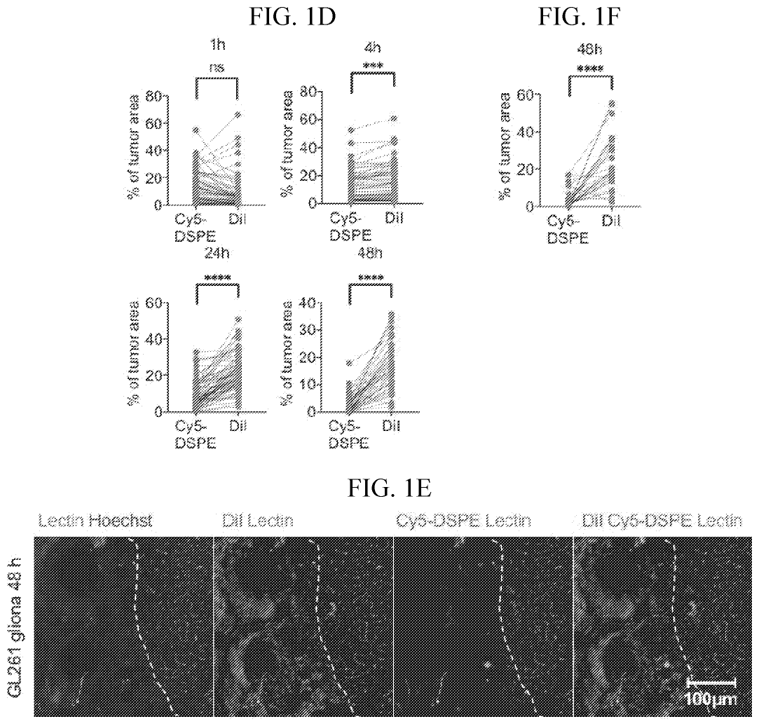

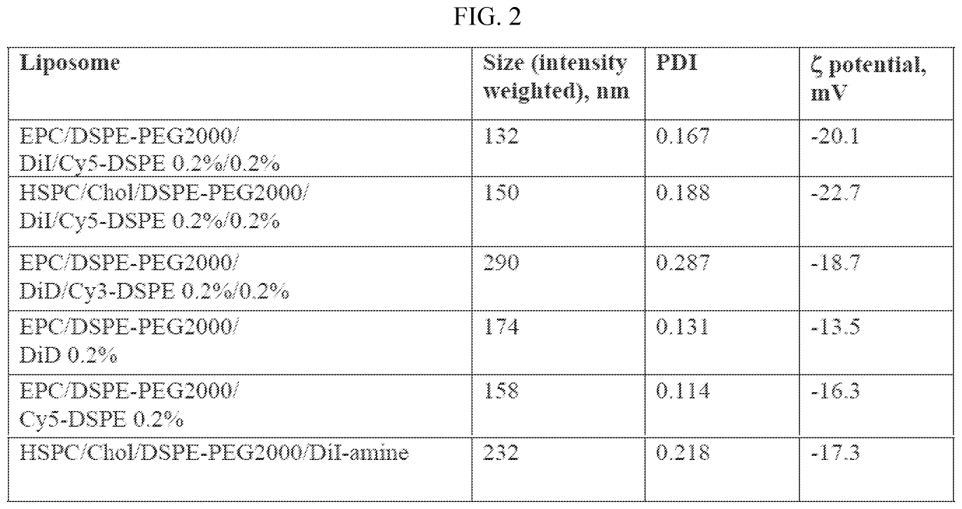

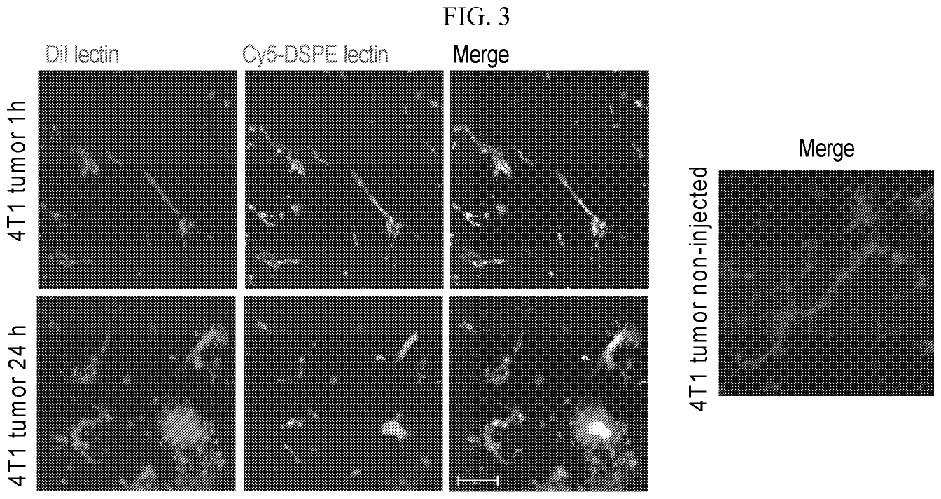

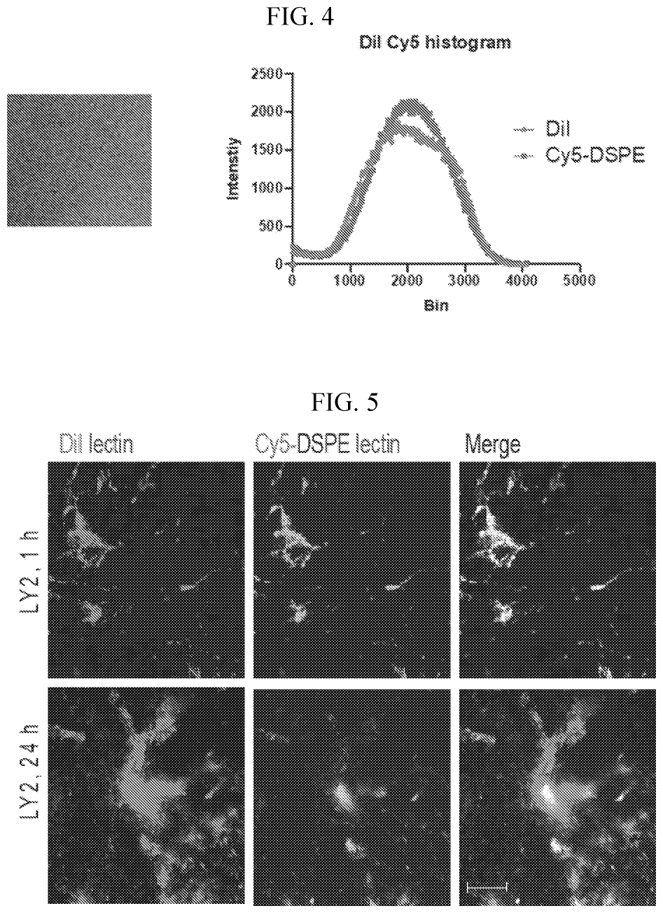

To compare tumor accumulation and extravasation of DiI and a phospholipid with a similar cyanine fluorophore headgroup but different excitation/emission spectra, Cy5-DSPE was synthesized (FIG. 1A). 130 nm, negatively charged egg phosphatidylcholine (EPC)/DSPE-PEG2000 liposomes were prepared with 0.2 mole % of DiI and 0.2 mole % of Cy5-DSPE in the same liposome (FIG. 2). High magnification confocal microscopy confirmed colocalization of both dyes in the same liposome, although the distribution of the dyes was not entirely homogenous (FIG. 1B). The liposomes were injected intravenously (i.v.) in 4T1 syngeneic orthotopic breast carcinoma-bearing mice. Confocal imaging of freshly excised, non-fixed (to avoid dye migration) tumor slices showed colocalization of both dyes in blood vessels at 1 h (FIG. 1C, upper panel and FIG. 3 for colocalization with blood vessel lectin staining). However, at 24 h post-injection, DiI efficiently extravasated and migrated throughout the tumor, whereas Cy5-DSPE was mostly confined to blood vessels (FIG. 1C, lower panel and FIG. 3 for colocalization with blood vessel lectin staining). Quantification of multiple confocal images taken in different areas of the tumor (gain settings were adjusted as described in Methods and FIG. 4) showed that while DiI and Cy5-DSPE occupied a similar area at 1 h post-injection, DiI occupied a significantly larger area than Cy5-DSPE at 4 h, 24 h and 48 h post-injection (FIG. 1D, p-value<0.0001 for all). To verify this difference in another immunocompetent subcutaneous model, DiI/Cy5-DSPE labeled EPC/DSPE-PEG2000 liposomes were injected in LY2 head and neck cancer and again observed colocalization of DiI and Cy5 in blood vessels at 1 h post-injection and much better extravasation of DiI at 24 h (FIG. 5).

Glioblastoma is the most aggressive and predominant type of gliomas with historical survival of only 20 months. One of the main limitations of the current therapies is the insufficient delivery of drugs to tumors due to low penetration through the blood-brain barrier (BBB) and blood tumor barrier (BTB). Accumulation of nanoparticles in gliomas is often imaged via ICL labeling. EPC/DSPE-PEG2000 liposomes labeled with 0.2% DiI and 0.2% Cy5-DSPE i.v. were injected in mice with the syngeneic intracranial GL261 glioma model. At 48 h post-injection, mice were perfused with FITC-lectin (blood vessel label) and Hoechst (nuclear label). According to ex vivo confocal microscopy imaging of fresh brain slices (imaging settings were adjusted as described in Methods), DiI accumulated and spread in tumors much more efficiently than Cy5-DSPE (FIGS. 1E-F). DiI visibly extravasated the lectin-positive tumor blood vessels and spread over a much larger area than Cy5-DSPE (FIGS. 1E-F, p-value<0.0001).

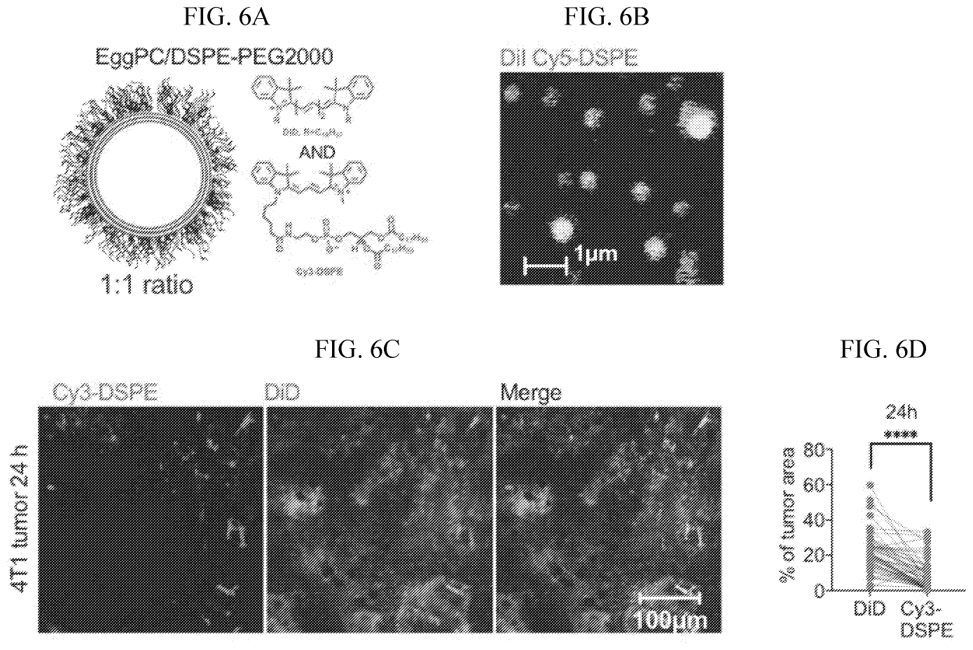

To confirm that the observed difference is not fluorophore-dependent and is not due to a selective quenching of Cy5-DSPE in tumors, colors were switched in the liposome. Cy3-DSPE was synthesized and co-formulated with DiD (FIG. 6A) at 0.2% each in 290 nm EPC/DSPE-PEG2000 liposomes (FIG. 2). Both dyes showed colocalization in liposomes under a high magnification objective (FIG. 6B). Twenty-four hours following i.v. injection in 4T1 tumor-bearing mice, DiD extravasated and migrated over a significantly larger area than Cy3-DSPE, which mostly stayed in blood vessels (FIGS. 6C-D, p-value<0.0001).

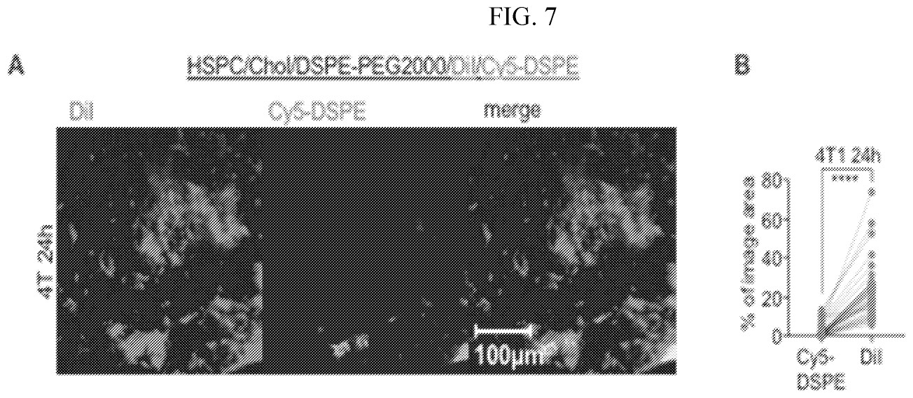

The experiments demonstrate that ICLs and FPLs exhibit significant differences in tumor extravasation and distribution at late time points. To validate these findings for other liposomal compositions, 150 nm negatively charged HSPC/Chol/DSPE-PEG2000 liposomes (DOXIL formula, FIG. 2) labeled with both 0.2% DiI and 0.2% Cy5-DSPE were prepared and injected i.v in 4T1 mice. Confocal microscopy imaging at 24 h showed the same result as for EPC/DSPE-PEG2000 liposomes, i.e., a more efficient extravasation and spreading of DiI than Cy5-DSPE (FIGS. 7A-7B).

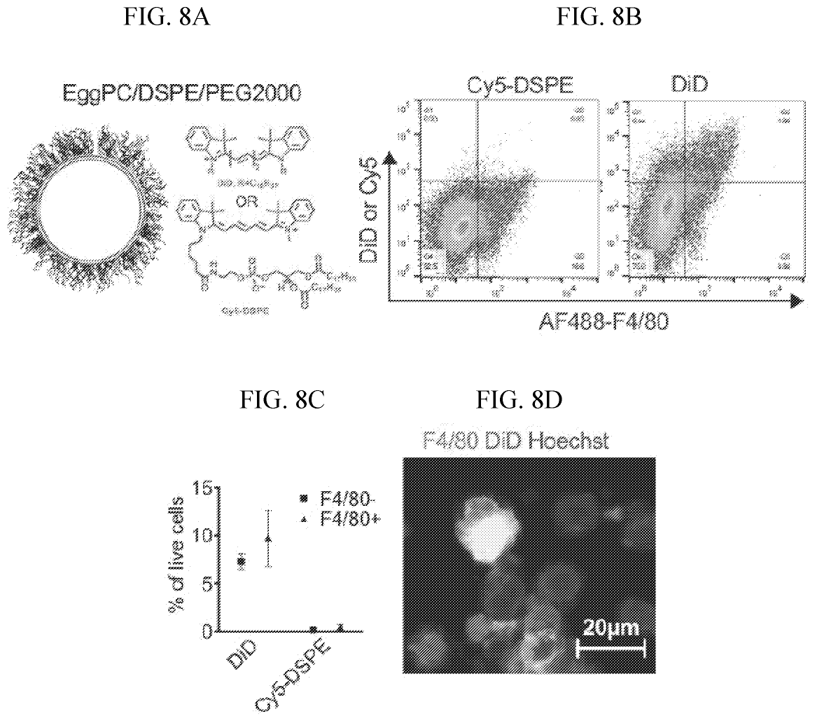

Previous observations demonstrated that nanoparticles and liposomes accumulate in the immune component of tumors. In some areas of 4T1 tumors, a macrophage-like accumulation pattern of lipids was observed (not shown). To measure accumulation in immune cells, liposomes separately labeled with 0.2% DiD or Cy5-DSPE were prepared (FIG. 8A and FIG. 2). Flow cytometry analysis 72 h after a single i.v. injection in 4T1 mice demonstrated minimal accumulation of Cy5-DSPE in cells in the tumor, whereas DiD liposomes resulted in about 17% of labeled cells (FIG. 8B), with over 50% of F4/80+ tumor-associated macrophages being positive (FIGS. 8C-D).

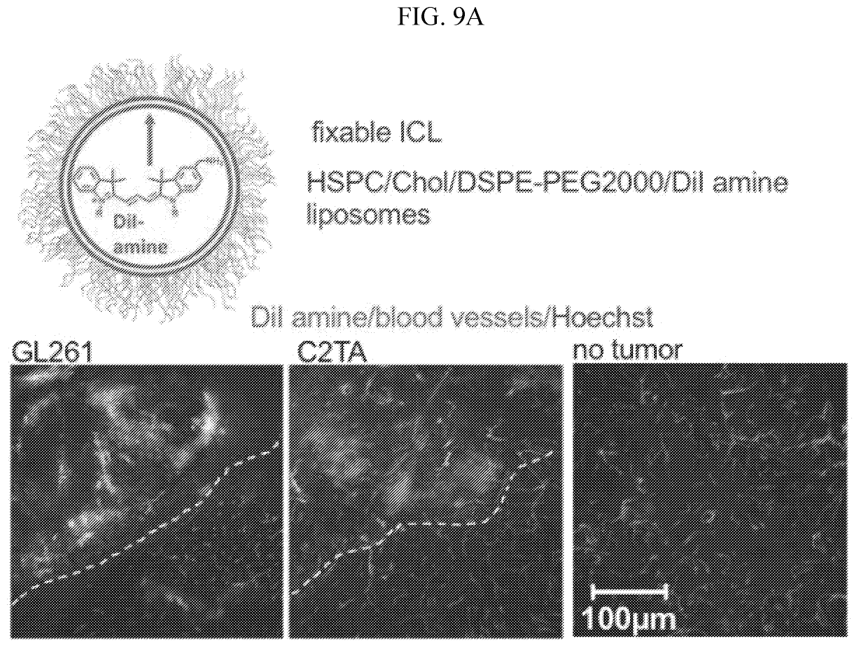

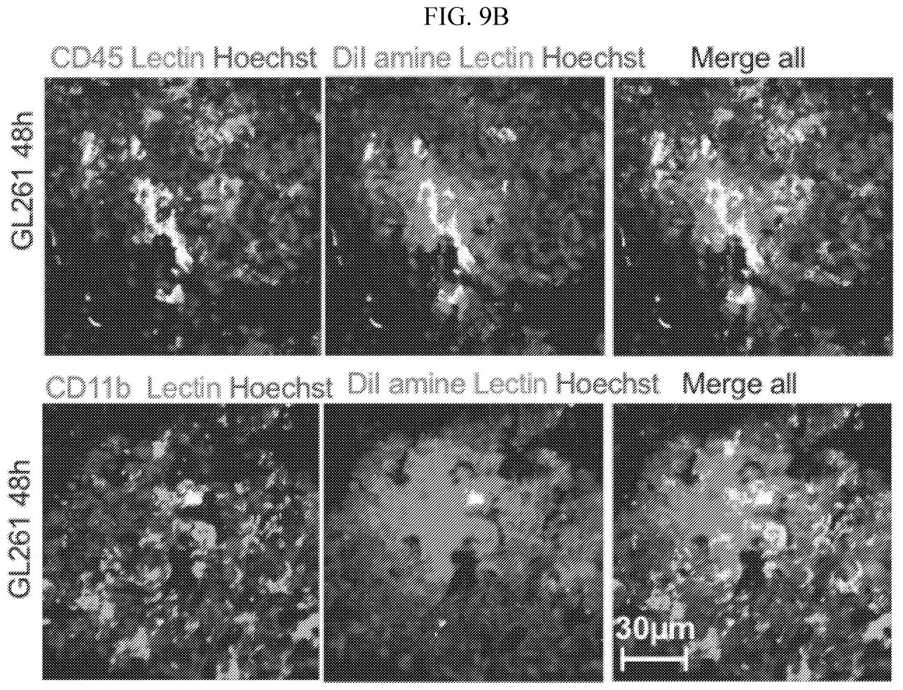

To characterize the immune uptake of ICLs in gliomas, immunostaining of GL261 tumors was performed. Since ICLs are non-fixable and migrate in tissues, liposomes labeled with a fixable aminomethyl derivative of DiI 41 were prepared (FIG. 9A and FIG. 2) and histological sections of GL261 tumors were made 48 h post-injection of DiI amine labeled liposomes. Colocalization of DiI amine with CD11b (myeloid) and CD45 (myeloid and lymphoid) markers was observed 48 h post-injection in GL261 mice (FIG. 9B). A proportion of DiI amine+ cells was negative for the immune markers. Overall, these findings suggest that ICLs are taken up by both immune and non-immune cells in tumors.

### ICLs and FPLs are Stable in Serum, and Extravasate Together in Tumors but Show Differences in Stability in Tumors and Tissues

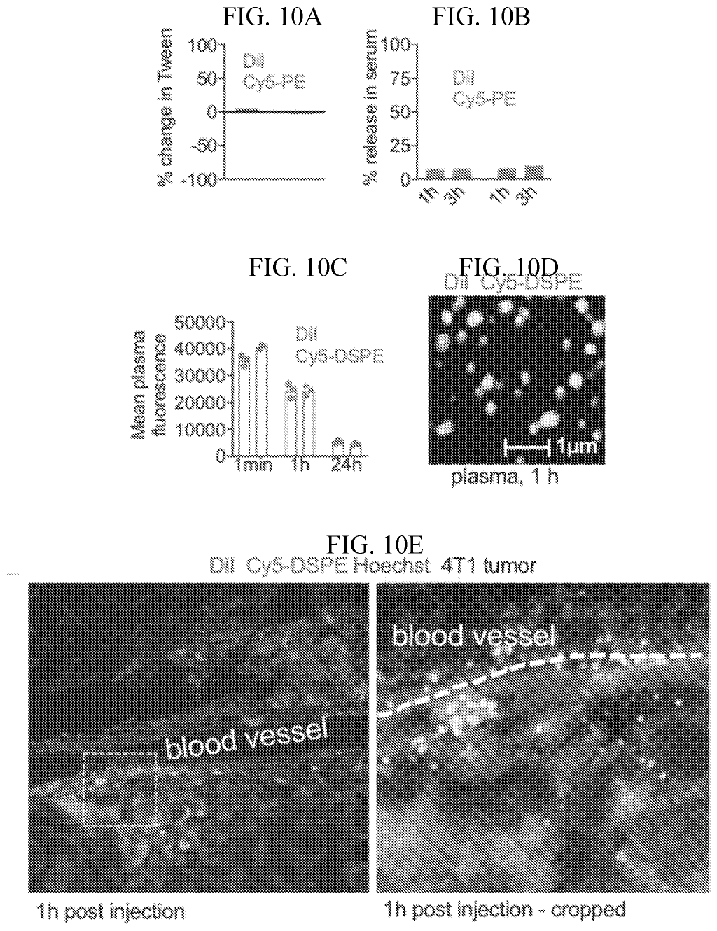

Intrigued by the data, the fluorescence behavior of DiI/Cy5-DSPE-labeled EPC/DSPE-PEG2000 liposomes was further investigated in vitro and in vivo. Both DiI and Cy5 fluorescence changed by less than 10% after incubation of liposomes in 1% Tween-20, suggesting that the fluorophores exhibit only a low level of quenching or energy transfer (FIG. 10A). It was next studied if the observed differences between ICLs and FPLs are due to the differences in stability of the dyes in liposomes in serum. Incubation of DiI/Cy5-DSPE liposomes in mouse serum showed less than 10% release of both DiI and Cy5 fluorescence after 1 h and 3 h (FIG. 10B). Furthermore, the elimination of DiI and Cy5 fluorescence from plasma were similar, with approximately 80% of both dyes being cleared from the circulation 24 h post-injection (FIG. 10C). Also, imaging of DiI/Cy5-DSPE liposomes in plasma 1 h post-injection showed mostly colocalization of both dyes in liposomes (FIG. 10D). To determine if the liposomes arrive in tumors intact, high-magnification confocal imaging of 4T1 tumor cryosections was performed (as opposed to low-magnification imaging of fresh tumor slices in FIGS. 1A-1F), 1 h post-injection. The images showed the binding of intact DiI/Cy5-DSPE liposomes to tumor endothelium and initial extravasation of DiI and Cy5-DSPE lipids (FIG. 10E).

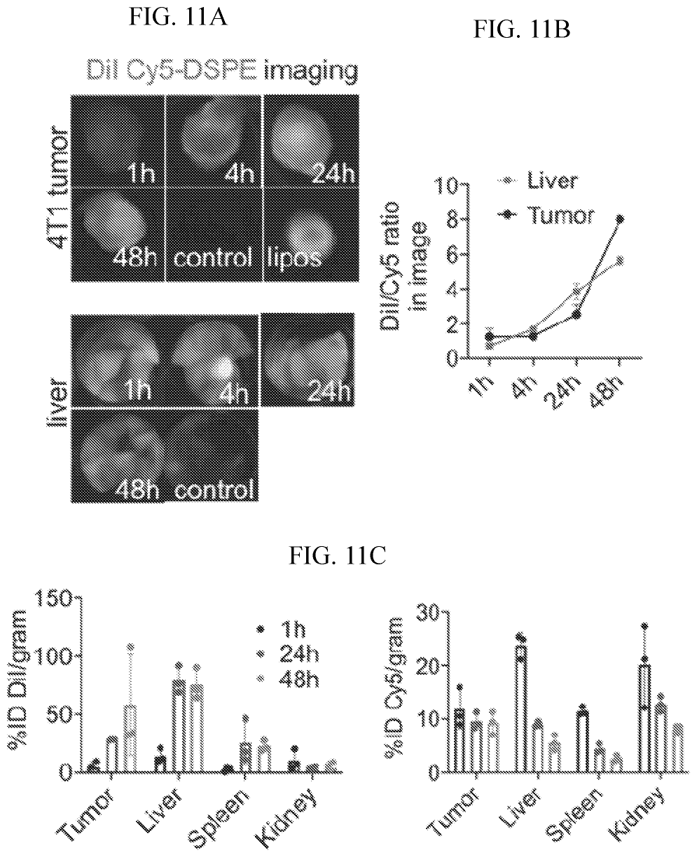

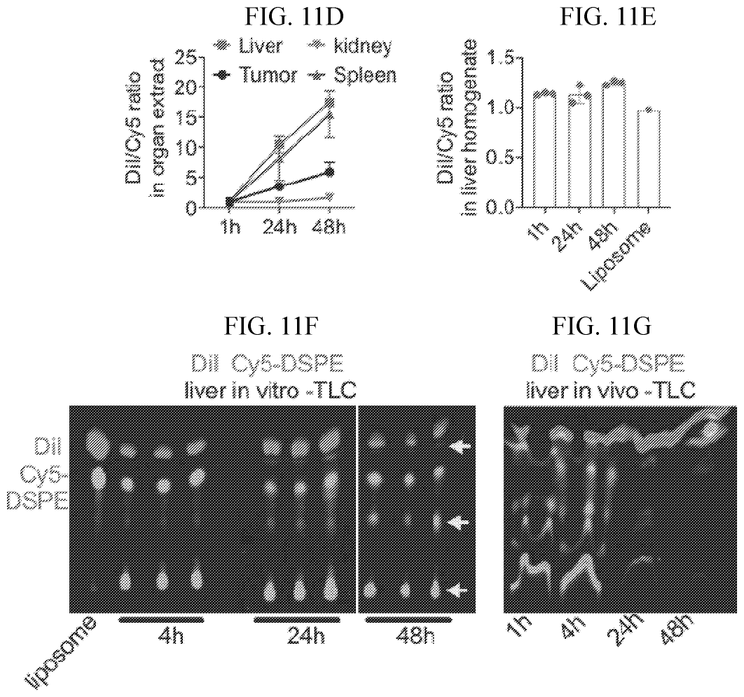

Since the accumulation of liposomes is often monitored via ex vivo organ imaging, DiI and Cy5 signals were imaged in freshly excised tumors and livers at different time points post-injection (gain settings were adjusted as described in Methods). The tumor images (FIG. 11A, upper panel) showed that both dyes accumulated to the same extent at 1 h. DiI fluorescence continued to increase at 24 h and 48 h, whereas Cy5 fluorescence plateaued at 24 h and dropped at 48 h (FIG. 11A). A similar trend was observed for the liver images (FIG. 11A, lower panel), which showed a similar accumulation of DiI and Cy5 signals at 1 h, but then an increase in DiI and decrease in Cy5 at later time points. Measurement of DiI/Cy5 fluorescence ratio in the images showed ˜6-fold increase over 48 h in both tumors and livers (FIG. 11B). These data are consistent with the confocal imaging of tumors and suggest that in both tumors and livers, there is an initial accumulation of ICLs and FPLs, followed by a decrease of FPL fluorescence. Fluorescence lipids are known to undergo quenching in different organs, including the liver. Therefore, a separate experiment was performed where the lipids from tumors, livers, spleens, and kidneys were extracted at 1 h, 24 h, and 48 h post-injection and remeasured the dye distribution (percent of injected dose/gram tissue) and DiI/Cy5 fluorescence ratio in the extracts. The organ distribution data in FIG. 11C showed drastic differences in the tumor and main organ accumulation of DiI and Cy5. Thus, while DiI showed an increase in all organs except the kidney over 48 h, the Cy5 amount dropped over time in all the organs. Notably, at 48 h post-injection, the DiI amount was highest in the tumor and liver, whereas the Cy5 amount was highest in the tumor and kidney. The DiI/Cy5 fluorescence ratio over time showed a much higher increase in the liver and spleen than tumor and kidney (FIG. 11E).

The data above demonstrate that Cy5 fluorescence, but not DiI fluorescence, gradually disappears from the tumor and other organs. This was especially prominent in the liver and spleen, which are the main organs that mediate the uptake of liposomes. To investigate whether Cy5 fluorescence is lost as a result of quenching or fluorophore metabolism, DiI/Cy5-DSPE liposomes were incubated in fresh liver homogenates for 1 h, 24 h and 48 h. According to FIG. 11E, there was no significant change in DiI/Cy5 fluorescence ratio compared to liposomes at all time points, suggesting no effect on the dye fluorescence. Thin layer chromatography analysis of the extracts of the liver homogenates showed the presence of both dyes at 1 h, but the appearance of degradation products of Cy5 at 24 h and 48 h (FIG. 11F). TLC analysis of the liver extracts of mice injected with DiI/Cy5-DSPE liposomes was performed and degradation and disappearance of Cy5-DSPE was observed, but not DiI at 24 h and 48 h (FIG. 11G), confirming that FPL is degraded and eliminated from the tissue.

### Selected Discussion

The mechanism of the EPR effect of nano-sized drug carriers is the fundamental question in drug delivery and is the subject of debate. The assessment of liposomal extravasation and distribution is based on fluorescent labels, most often using lipophilic membrane dyes. The significance of the present work is that, using 2 main classes of lipid dyes incorporated in the same PEGylated liposome, it was demonstrated for the first time that depending on the label, a different conclusion can be made about the efficiency of the extravasation and accumulation in tumors as well as other organs. Thus, while ICLs and FPLs show similar extravasation at early time points, ICLs exhibited significantly better extravasation, migration, and retention at later time points. Artifacts were excluded by a) using the probes with the similar fluorescence properties and extinction coefficients due to the same class of cyanine headgroup; b) labeling of liposomes at 1:1 molar ratio; c) calibrating the intensity of Cy3 and Cy5 channels for confocal microscopy and imaging using the liposomal batch used in each experiment. Moreover, this phenomenon was observed regardless of the fluorophore wavelength, liposome type, and tumor location (breast, head and neck, and brain). Collectively, the data suggest that the decrease in the FPL fluorescence in tumors is not due to selective quenching of FPLs, but rather due to the degradation and elimination. This process was not specific to tumors, and while the studies focused on the liver, spleen, and kidneys as the main clearance organs, it took place in other organs (not shown). One possible hypothesis for this phenomenon is as follows. Once in the tissues, most likely inside the cells, liposomal phospholipids could be subject to metabolism and degradation, for example by phospholipases, as well as due to acidic hydrolysis leading to release of glycerol phosphoethanolamine and lysophospholipids. FPLs could undergo the same fate, with smaller fluorescent fragments subsequently being washed out of the tissues and tumor. While LC-MS analysis of tissue extracts was not feasible at this point due to the large excess of other lipids and hydrophobicity of ICLs and FPLs, the degradation of FPLs in liver homogenates in vitro and liver extracts in vivo was observed by TLC. It is hypothesized that similar processes could happen in tumors. The uptake of liposomes and nanoparticles by the tumor's immune component is well established, and an efficient uptake by tumor macrophages and immune cells was observed in both 4T1 and gliomas (FIGS. 8A-8D and FIGS. 9A-9B). The uptake by tumor macrophages could lead to efficient degradation of FPLs in lysosomes while retaining more stable ICLs.

The data suggest that liposomes do not stay intact in tumors and different components of liposomes separate and are subject to a different fate. The studies are underway to understand the differences in migration of liposomal payload and the lipid and to understand the mechanism whereby liposomes cross the endothelial barrier and are taken up by the tumor microenvironment. It was concluded that different liposomal labels could lead to different conclusions about tumoral extravasation and trafficking and biodistribution as studied by microscopy and imaging. It is therefore recommended that caution should be exercised in interpreting biodistribution and tumor extravasation of nanocarriers based on indirect labeling.

### Example 2: Nanoformulated Indocarbocyanine Lipids Exhibit Efficient Extravasation and Targeting of an Immune Microenvironment and Invasive Edge in Gliomas

Among solid tumors, gliomas are the most devastating and aggressive brain tumor in adults and children, with a median survival of only 14.6 months. Hidden behind the blood-brain (BBB) and blood-tumor barriers (BTB), gliomas, especially at the invasive edge, are not readily accessible to the majority of drug therapeutics. Here we found that fluorescent indocarbocyanine lipids (ICLs, DiI, DiD) formulated in PEGylated liposomes (300 nm, 0.4 mol % ICL) or PEGylated nanoparticles (530 nm, 20 mol % ICL) efficiently cross the BTB/BBB and extravasate in multiple syngeneic models of glioma. As soon as 1 h post injection of nanoformulated ICLs, they extravasated via micron-sized particles, some of them budding from the abluminal part of tumor endothelial cells, but also via diffusion of lipid away from blood vessels. At 48 h post-injection, as much as 70% of tumor area was positive for ICLs. Following extravasation, ICLs efficiently accumulated in immune cells, predominantly microglia, tumor-associated macrophages, myeloid-derived suppressor cells, dendritic cells, regulatory T cells, and natural killer cells, but also in non-immune cells. Almost 100% of myeloid cells and 70% of non-immune (CD45−) cells were ICL-positive. The uptake by myeloid-derived suppressor cells, tumor-associated macrophages and dendritic cells was complement-dependent. When co-injected with PEGylated liposomal doxorubicin, nanoformulated ICLs labeled more cells inside of the tumor and at the invasive tumor edge than doxorubicin. Understanding the mechanism of extravasation of ICLs can lead to improved approaches to systemic delivery to invasive gliomas.

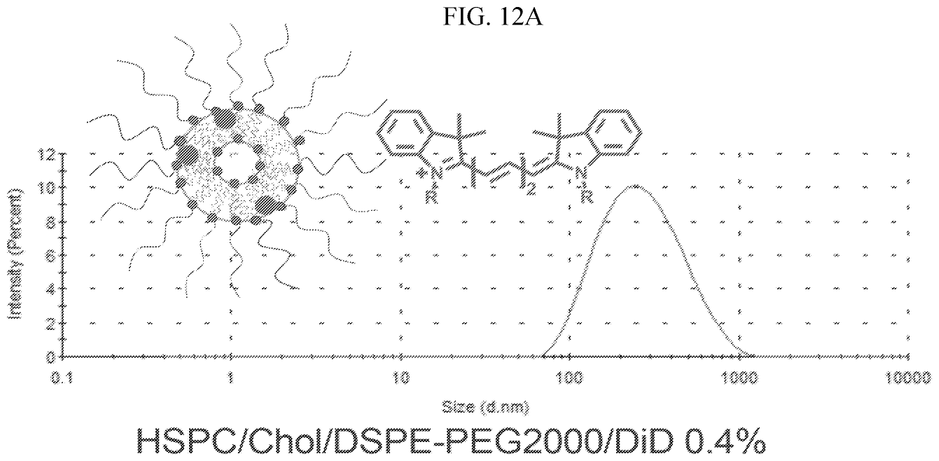

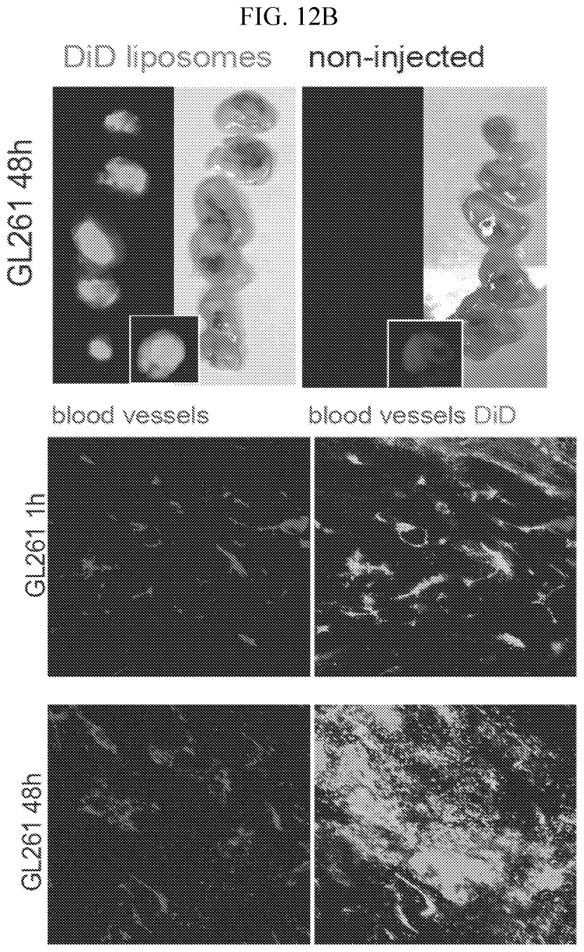

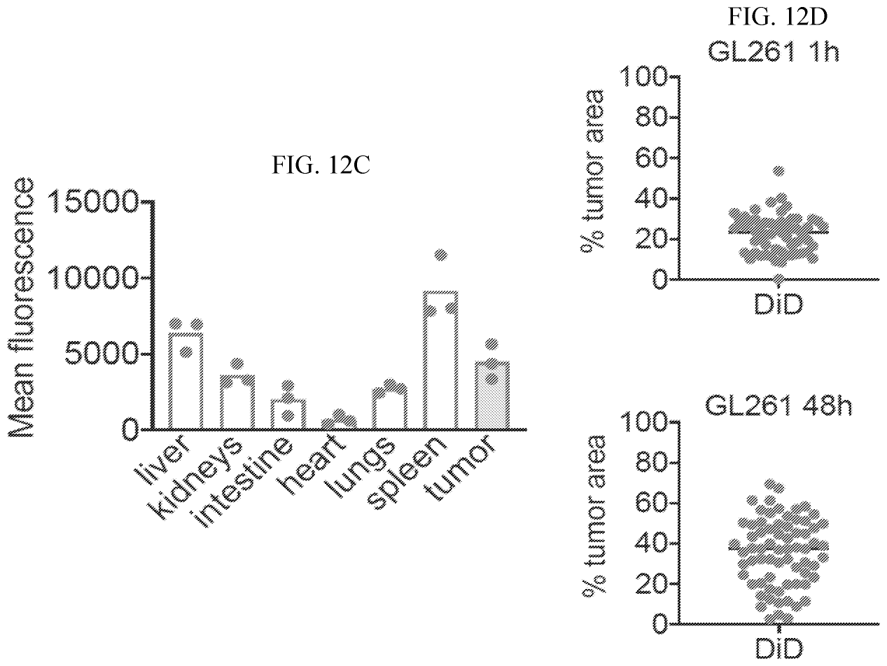

FIG. 12A depicts liposomal ICLs size FIG. 12B depicts extravasation and accumulations of ICLs in glioma when formulated in liposomes.

FIG. 12C depicts biodistribution and extravasation of liposomal ICLs in glioma.

FIG. 12D depicts spreading of ICLs after injection of liposomes in GL261 glioma mice.

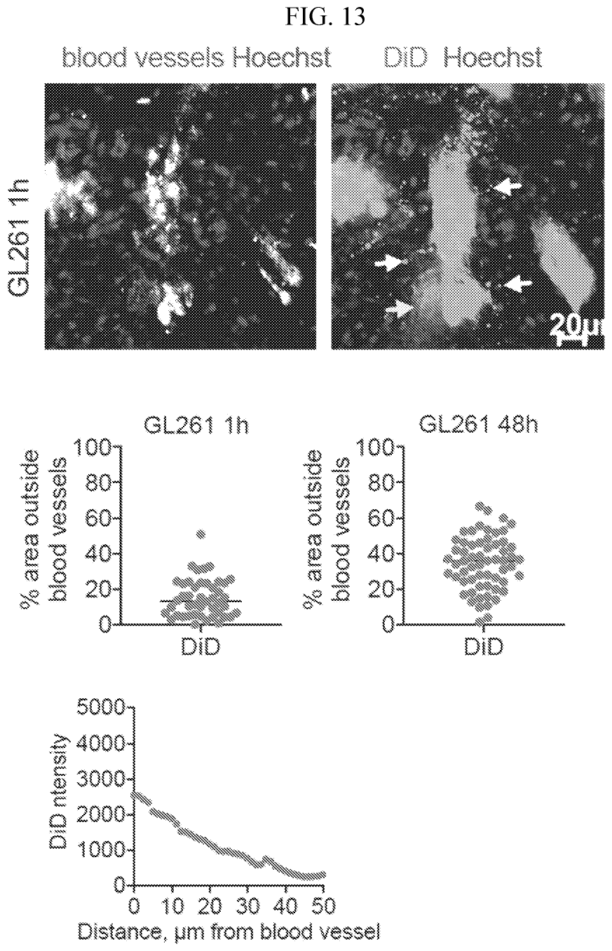

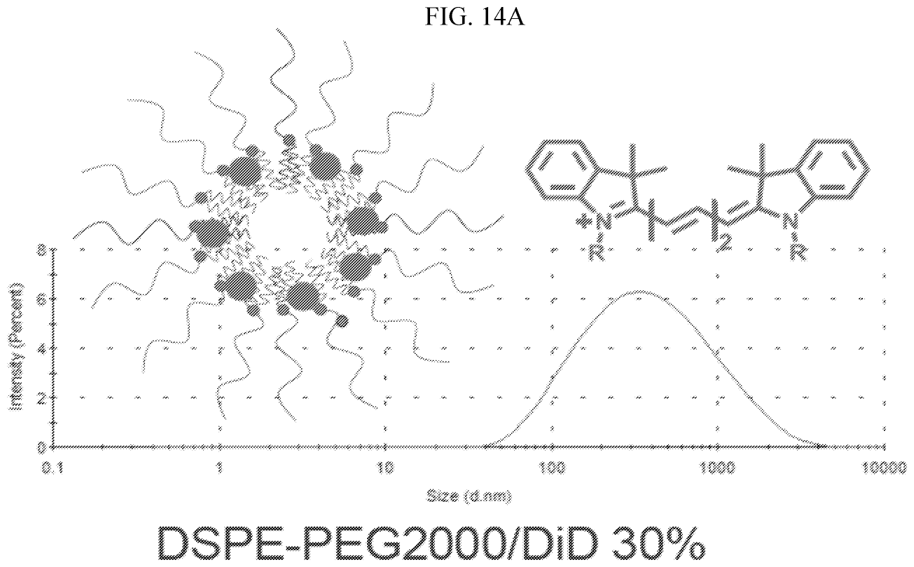

FIG. 13 depicts extravasation—GL261, DiD liposomal.

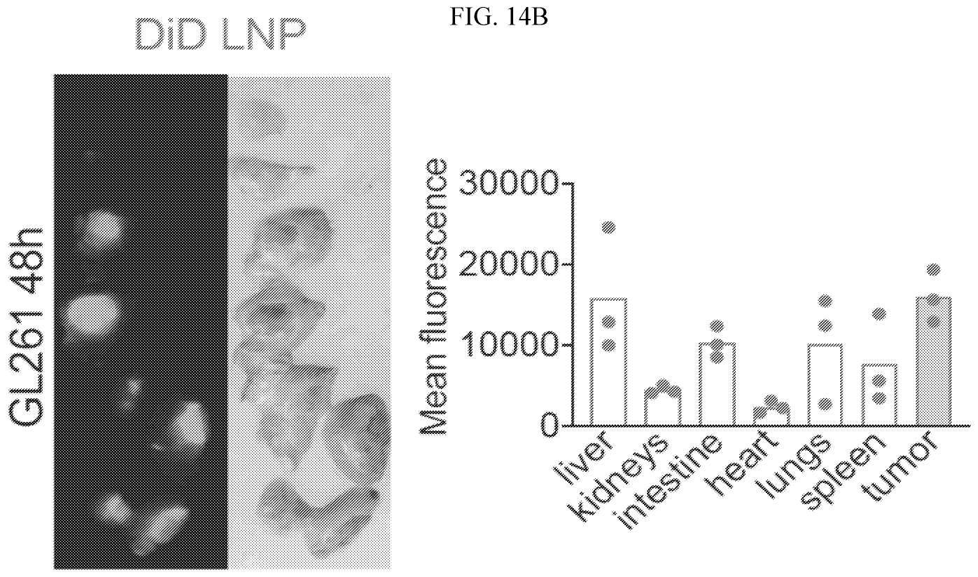

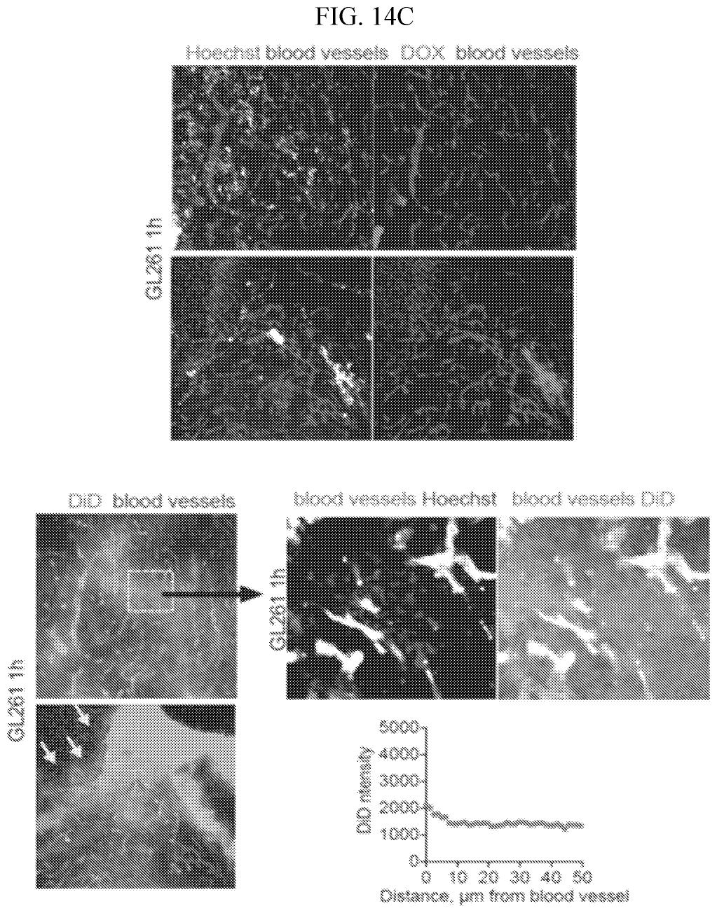

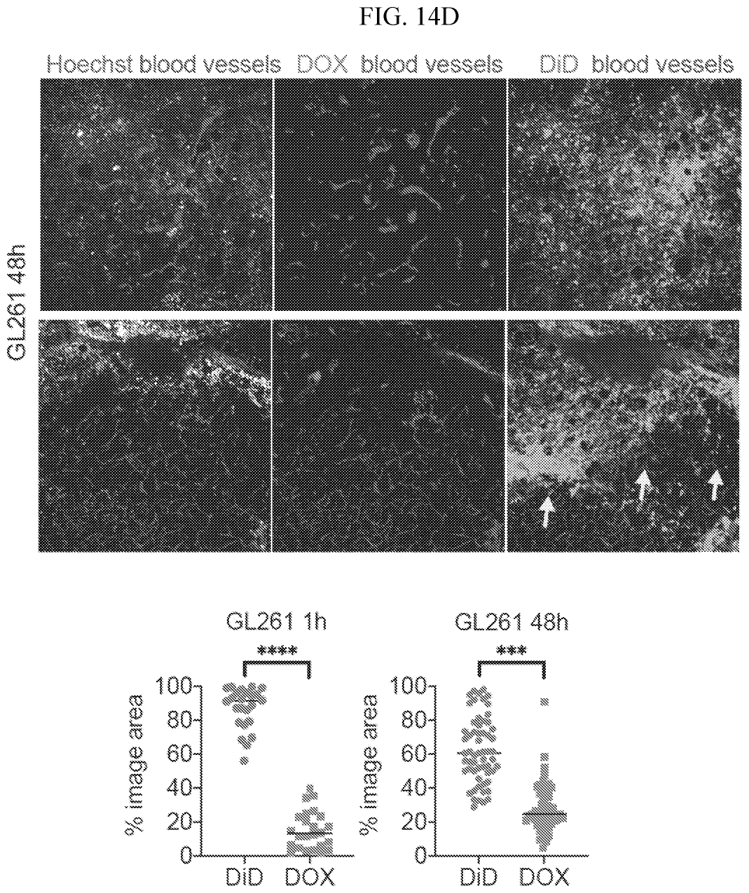

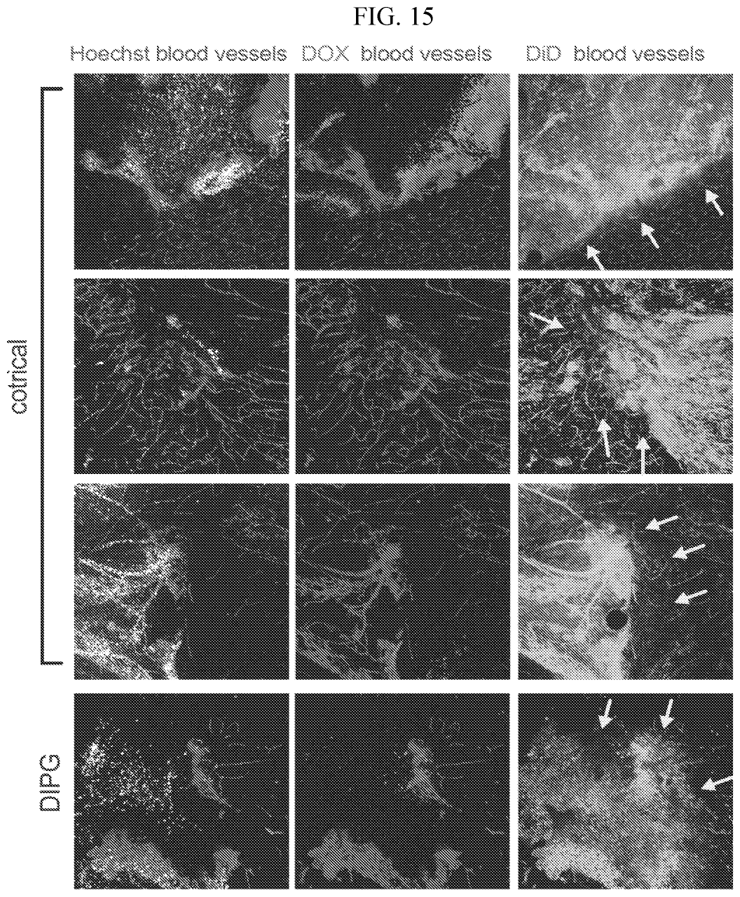

FIG. 14A depicts lipid nanoparticle (DSPE-PEG2000/DiD) size.

FIG. 14B depicts biodistribution in organs and glioma.

FIGS. 14C-14D depict extravasation of ICLs formulated in lipid nanoparticles are injected in GL261 glioma.

FIG. 15 depicts that lipid nanoparticles ICLs show better extravasations and spreading than PEGylated liposomal doxorubicin. FIG. 15 further depicts extravasation of lipid nanoparticles ICLs in invasive glioma models showing accumulation in the invasive edge.

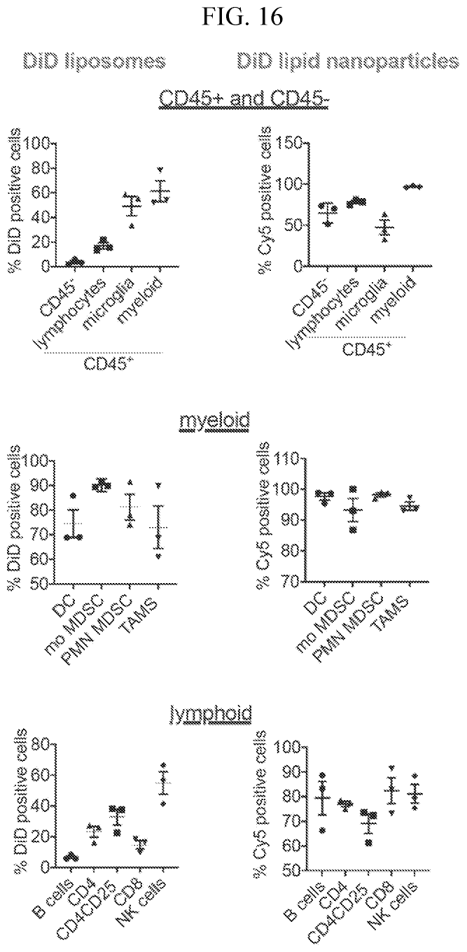

FIG. 16 depicts flow cytometry of cells that take up liposomes or lipid nanoparticles in glioma.

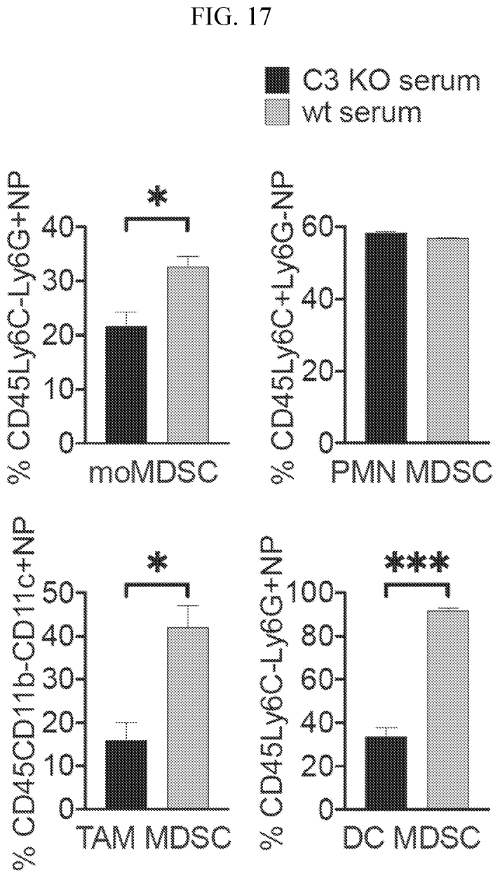

FIG. 17 depicts that complement plays a role in the uptake of ICLs by immune cells in glioma.

### Example 3: Fluorescent Indocarbocyanine Lipids for Understanding and Overcoming

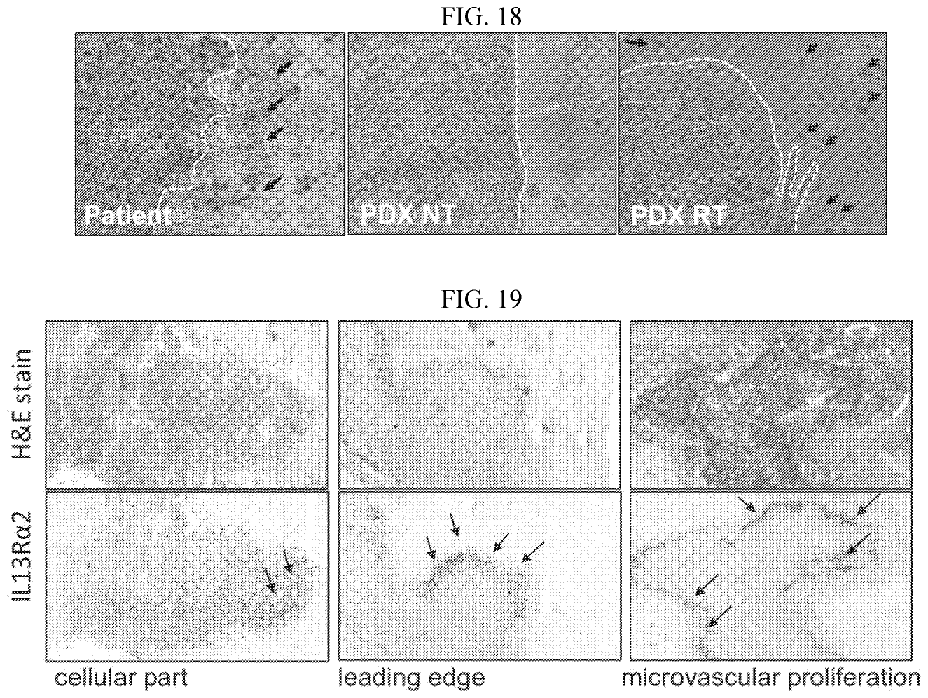

Barriers to Drug Delivery in Glioblastoma Glioblastoma is the most aggressive and predominant type of astrocytomas with historical survival of only 20 months. GBM does not generally metastasize but is characterized by invasive phenotype, in which cells migrate in the brain away from the main tumor mass, often along of myelinated nerve tracks and blood vessels (FIG. 18). Radiation therapy (RT) combined with surgery and chemotherapy is the backbone of glioma therapy. While RT leads to vascular damage and often increases tumor permeability, it also increases invasiveness (FIG. 18).

Despite the advances in the understanding of glioma biology, the majority of novel therapies, including molecularly targeted drugs, angiogenesis inhibitors, and immunotherapies, result in only a modest increase in patient survival and inevitable progression. One of the main limitations of the current therapies is the insufficient delivery of drugs to tumors due to poor BBB and BTB penetration. While tumor progression leads to BTB structural changes, including neuronal death, astrocyte end-feet displacement, and heterogeneous pericyte subpopulations, the accumulation of drugs in brain tumors is heterogeneous. Several strategies to improve drug delivery include convection-enhanced delivery and focused ultrasound-mediated disruption of the BBB and BTB, with mixed results in the clinic. Preclinical and clinical evidence suggests that improving BBB/BTB penetration is critically needed to achieve therapeutic delivery to the invasive cells.

Given these limitations, nanoparticles have been suggested as an attractive solution to improve the penetration of drugs to gliomas. Unfortunately, the delivery of nanoparticle (NP)-based agents to tumors, including gliomas, is still plagued by poor penetration, diffusion, and retention, leading to the low clinical efficacy of conventional nano drugs. Various chemistries, nanoparticle types, and targeting ligands have been tested for improving trafficking across BBB and BTB in gliomas. Several studies with NPs targeted to angiogenesis and endothelial transporters demonstrated improved extravasation and accumulation in gliomas. Despite the gain in tumor accumulation and therapeutic efficacy through engineering, limited understanding of mechanisms by which nanoparticles extravasate the BTB and spread in gliomas and lack of focus on targeting clinically-relevant invasive cells hamper qualitative improvements in treatment.

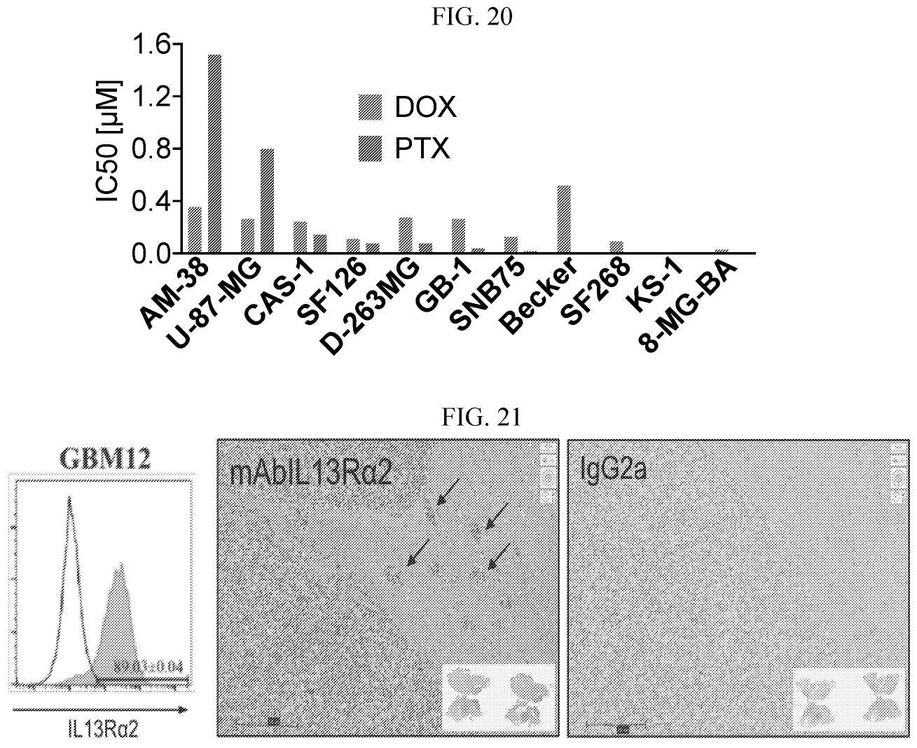

IL13Rα2, the high-affinity IL13 receptor, is an ideal therapeutic target due to its frequent high-level expression in GBM cells but not in normal tissues, and its expression is associated with a poor prognosis. IL13Rα2 is highly expressed on the invasive edge of the gliomas (FIG. 20). IL13 ligand-based approaches to target IL13Rα2 are not feasible due to ligand binding to ubiquitously expressed IL13Rα1. To overcome this limitation in exploiting IL13Rα2 as a therapeutic target, an IL13Rα2 scFv was developed from a parental monoclonal antibody, which binds to IL13Rα2 but not IL13Rα1. Using this scFv as the targeting moiety, IL13Rα2-specific CAR T cells were developed and it was validated that these cells recognize and kill only IL13Rα2-positive cells but not IL13Rα1-expressing cells. IL13Rα2-CAR T cells induce tumor regression in orthotopic xenograft and murine models of GBM, resulting in significant survival extension. An additional example using the scFv for treating GBM is a study in which an adenovirus was modified by incorporating the scFv as the targeting moiety in the viral fiber. It was shown that this modification successfully directed adenovirus to IL13Rα2-expressing GBM cells both in vitro and in vivo. Most recently, the scFv has been humanized (not published data), making it a valuable targeting agent for clinical applications. Collectively, the data support using this scFv as a highly selective IL13Rα2 targeting agent and has served to motivate further exploration of its use in developing IL13Rα2-targeted therapeutics. Having identified high expression of IL13Rα2 in the invasive edge of tumor, the targeting approach using scFv IL13Rα2 addresses the major problem in GBM recurrence.

### Methods

### Preparation of ICL-Drug and ICL-Antibody Conjugates

Cleavable and non-cleavable conjugates of doxorubicin (DOX) and paclitaxel (PTX) will be prepared. Both drugs are highly toxic to glioma cells in the nanomolar range (FIG. 21), but do not penetrate through the BBB/BTB. DOX is very convenient as it can be visualized directly by fluorescence, while PTX can be visualized by immunostaining. PDX GBM models recapitulate high expression of IL13Rα2 at the invasive edge and by satellite tumors (FIG. 19 and FIG. 21). While targeting does not necessarily increase net accumulation of nanoparticles, it increases intracellular accumulation and changes distribution toward tumor cells. The successful delivery of these potent drugs to GBM could increase a very limited repertoire of therapeutics to patients.

Self-immolating disulfide chemistry has been used for slow release by the intracellular reducing environment, in antibody-drug conjugates and prodrugs of doxorubicin and paclitaxel. Aminomethyl DiD will be synthesized according to a previously published procedure. The amino group of DiD will be conjugated via a disulfide linker to the secondary alcohol group of paclitaxel or to the primary amine of doxorubicin (FIGS. 22A-22B). For antibody conjugation, copper-free bio-orthogonal click chemistry involving strained trans-cyclooctene (TCO) and methyl tetrazine (MTz) will be used. DOCy7-PEG-MTz (analog of DiR) will be used in order to conjugate TCO-modified scFv against IL13Rα2 (FIG. 22C).

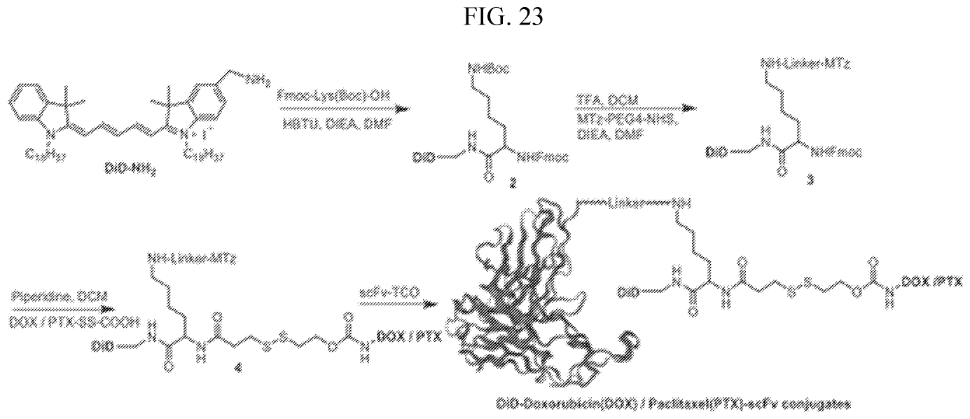

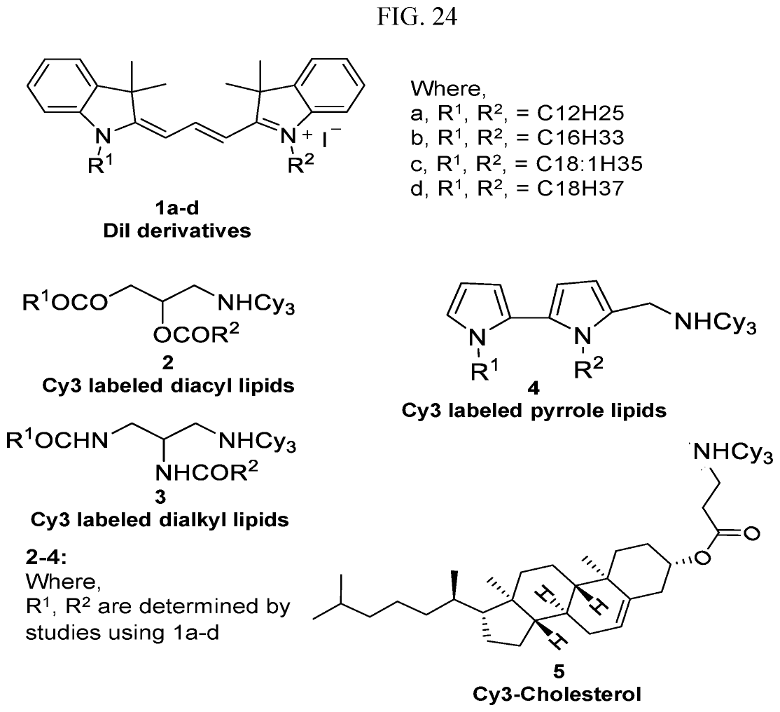

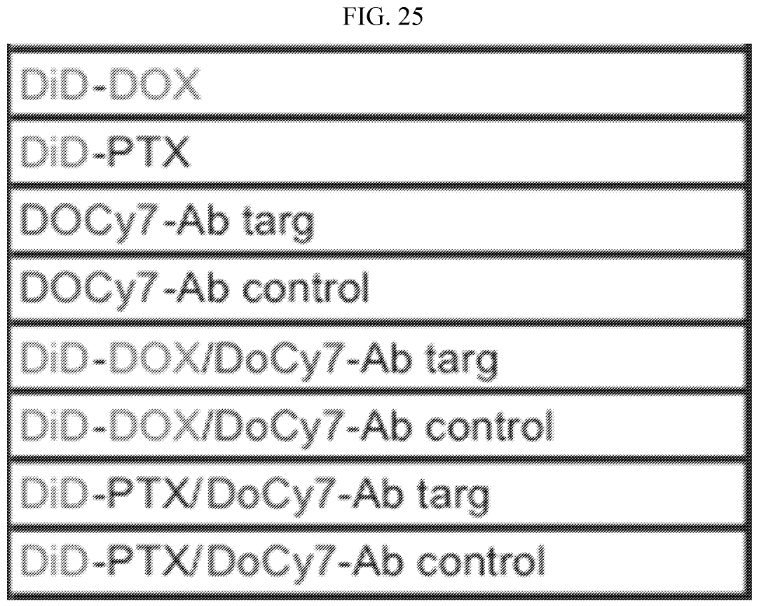

Dual ICL-drug/antibody conjugates, as exemplified in FIG. 23, will also be synthesized. Additionally, if the C18 lipid of ICLs is not be optimal, other lipids (FIG. 24) will be synthesized.

Anti-Human IL13Rα2 scFv Preparation

The antibody and derived scFv anti-human IL13Rα2 is well validated for its affinity and specificity of binding by glioma cells. For use as a negative control, which does not bind to IL13Rα2, scFv IL13Rα2 was developed in which the light chain complementarity-determining region 3 (CDR3) of scFvIL13Rα2 was replaced with the CDR3 domain of non-specific antibody MOPC1 (p588). In order to enable conjugation to ICLs, a cysteine in the linker region will be introduced. The 293T cells transduced with a lentiviral vector encoding for the scFv IL13Rα2 or its negative control will be cultured in DMEM supplemented with 10% FBS. For a collection of secreted scFvs, 293T/17 cells (ATCC) will be incubated in OptiMEM serum-free media in the presence of protease inhibitors (Sigma-Aldrich) in a CO₂ incubator at 32° C. Supernatants containing scFv proteins will be collected, filtered through 0.4 μm filter, and affinity-purified using HisPure columns. Purified proteins will be dialyzed against PBS, aliquoted, and stored at −80° C. until needed.

### Formulation and In Vitro Testing of the Conjugates

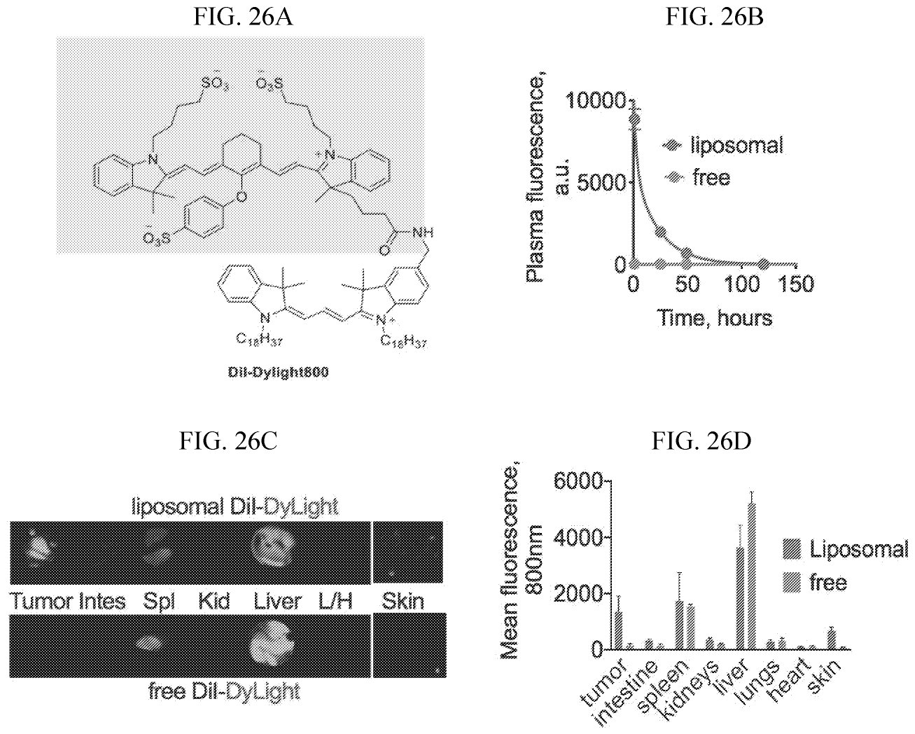

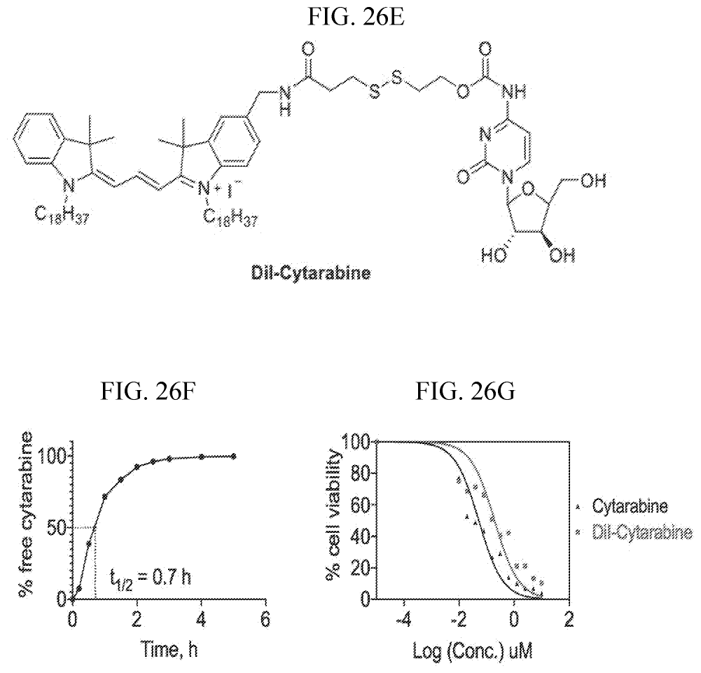

Fluorescent ICLs enable “mix and play” approach wherein different-colored conjugates can be traced simultaneously (FIG. 26). Helper lipids, DiD-drug, and DoCy7-PEG3400-MTz, will be mixed in ethanol, diluted to 10 mM total lipid, and dialyzed against sterile saline. The antibody will be conjugated to the particles, according to FIGS. 22A-22C. The engineered cysteine of scFv will be reacted with TCO-PEG3-maleimide, followed by a click reaction to MTz. Click reaction is highly versatile due to very fast second-order kinetics and the high stability of the resulting bond; in our previous studies, over 50% conjugation efficiency was found.

The unconjugated antibody will be separated over the Sephacryl-5300 HR column (Cytiva Life Sciences). Size, zeta potential and serum stability will be tested. For uptake, different concentrations of targeted and non-targeted particles will be added to GBM12 PDX cells that express IL13Rα2 (FIG. 21) or murine glioma GL261 genetically modified to express IL13Rα2 for 1-6 h, and the binding will be quantified with flow cytometry. A GBM39 model that does not express IL13Rα2 or non-modified murine glioma, GL261, and CT-2A, cells will be used as a negative control. The drug release from the conjugates will be determined in different concentrations of DTT, from 10 μM to 10 mM, over 24 h, as previously described and in FIG. 26F. Cytotoxicity (IC₅₀ values) will be determined in the MTT assay and compared to the free drug. Based on these studies, lead conjugates will be selected for in vivo studies.

### Results

ICL Headgroup can be Conjugated with a Payload for Drug Delivery

In recent publications, the synthesis and characterization of ICL derivatives for modification of cell membranes was described. To this end, aminomethyl derivatives of DiI and DiR were synthesized and covalently conjugated to antibodies, enzymes, PEG, and small molecules. To test the proof of principle of drug delivery using a model payload, a NIR dye, DyLight800, was covalently conjugated to aminomethyl DiI (FIG. 26A). DiI-DyLight was formulated in EggPC/DSPE-PEG2000 liposomes at 0.5 mole % or injected as a free conjugate. The conjugate in PEGylated liposomes showed longer circulating properties (FIG. 26B) and significantly more accumulation in the 4T1 tumor than the free conjugate (FIGS. 26C-26D). Tumors, spleen, and liver showed the most accumulation. There was much less accumulation in the intestine, kidneys, skin, heart, and lungs (FIGS. 26C-26D). A cleavable (self-immolating disulfide bond) conjugate of DiI-cytarabine was prepared (FIG. 26E) which demonstrated drug release by HPLC and efficient cytotoxicity in leukemic cells in vitro (FIGS. 26F-26G). These data establish that ICLs can be used for drug delivery.

### Example 4: ICL-Drug and ICL-Drug-Antibody Conjugate

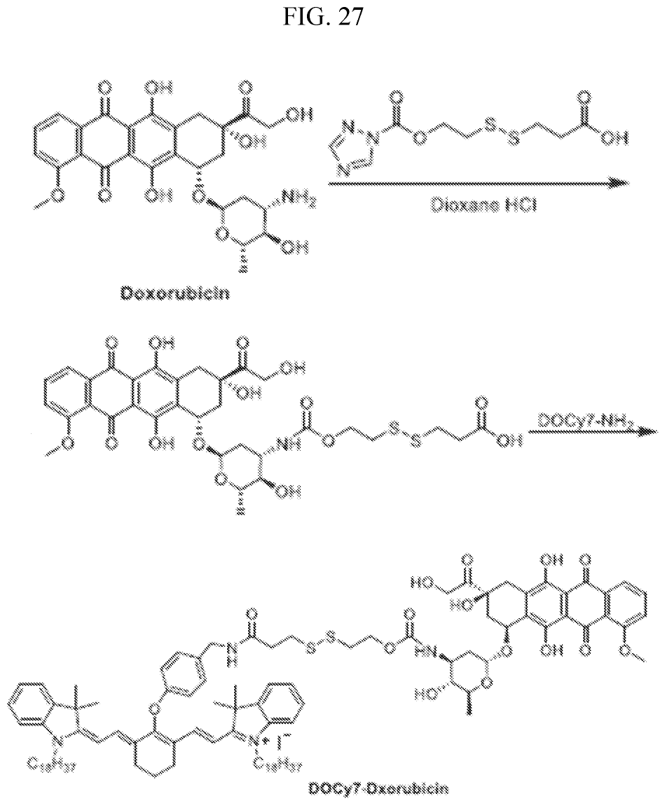

FIG. 27 depicts an example of synthesis of a lipid-doxorubicin conjugate.

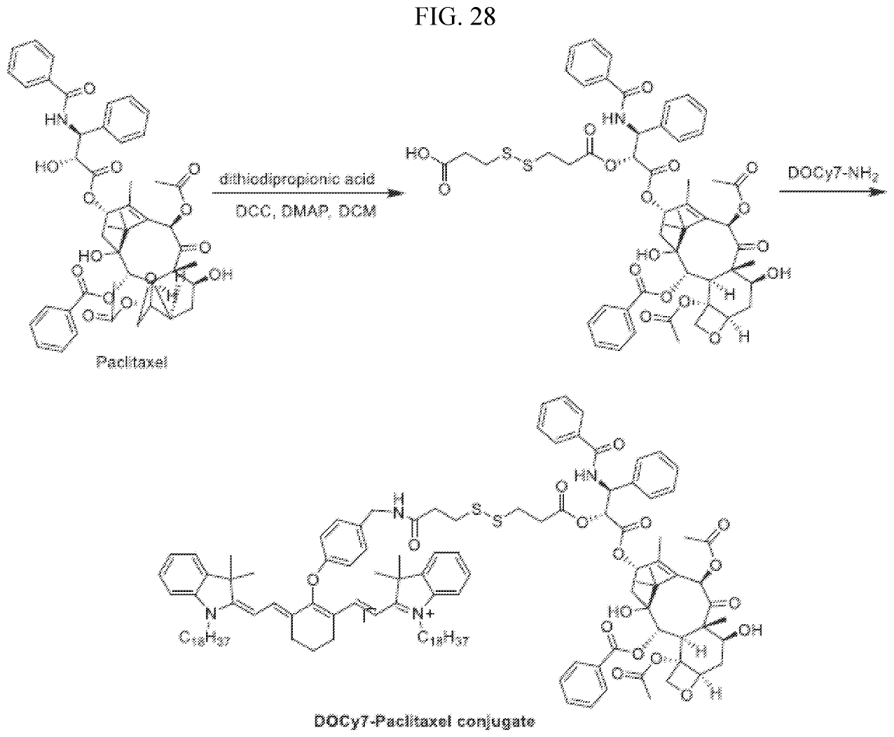

FIG. 28 depicts an example of synthesis of a lipid-paclitaxel conjugate.

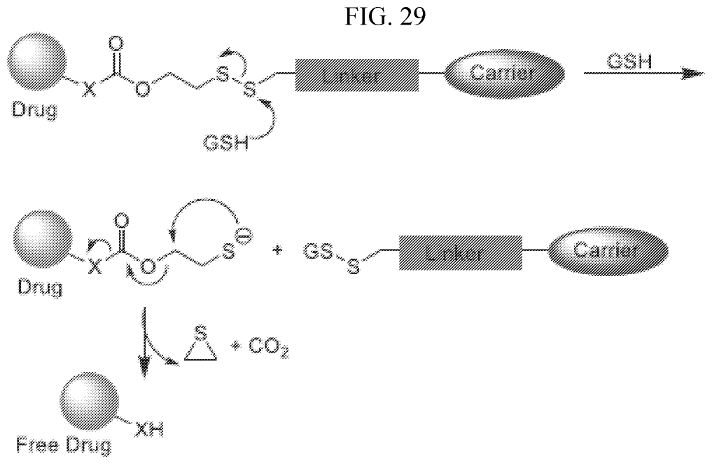

FIG. 29 depicts an example of self-immolating release from a lipid-drug conjugate.

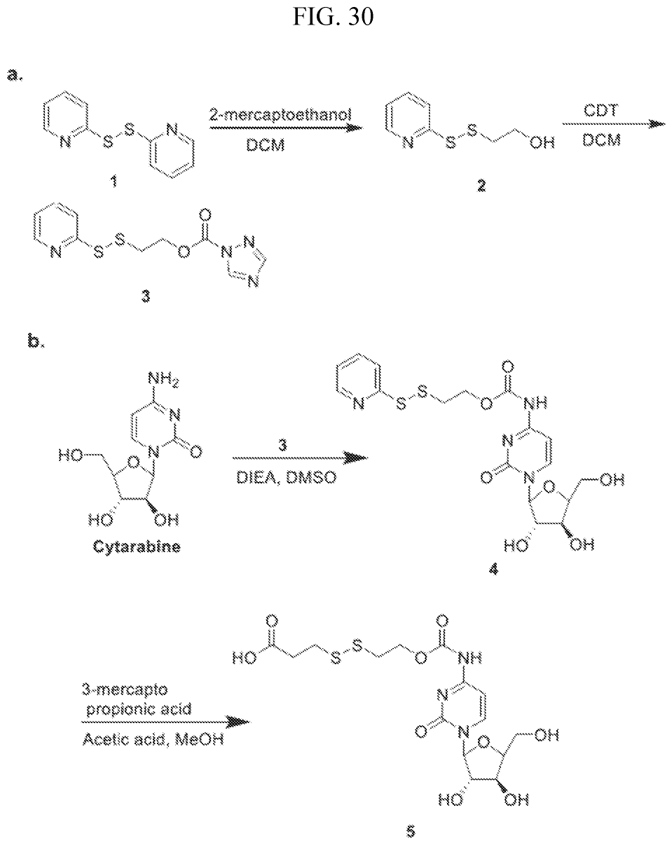

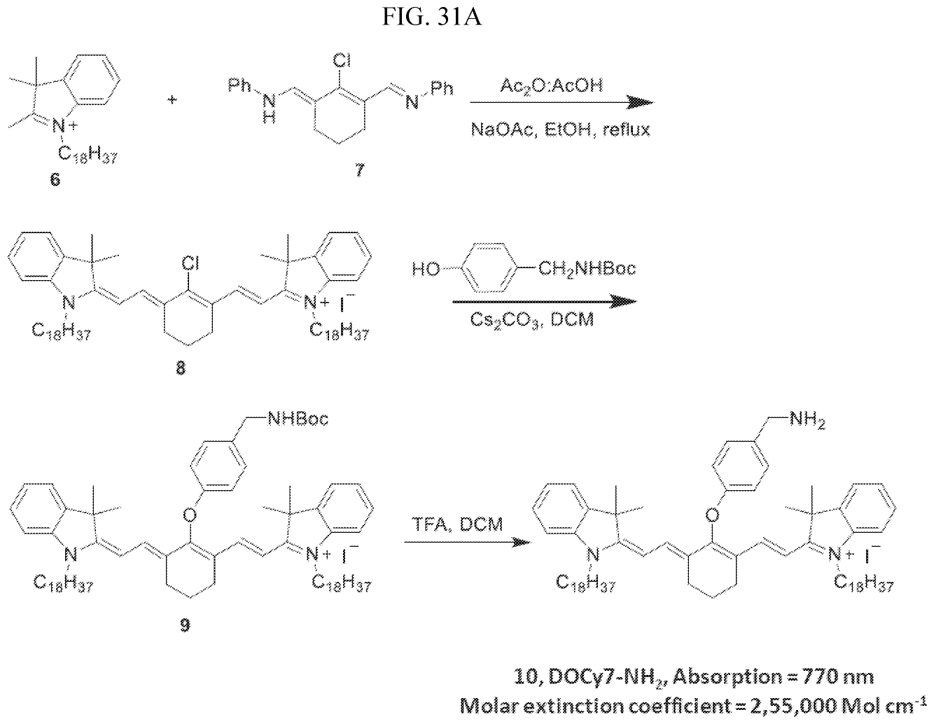

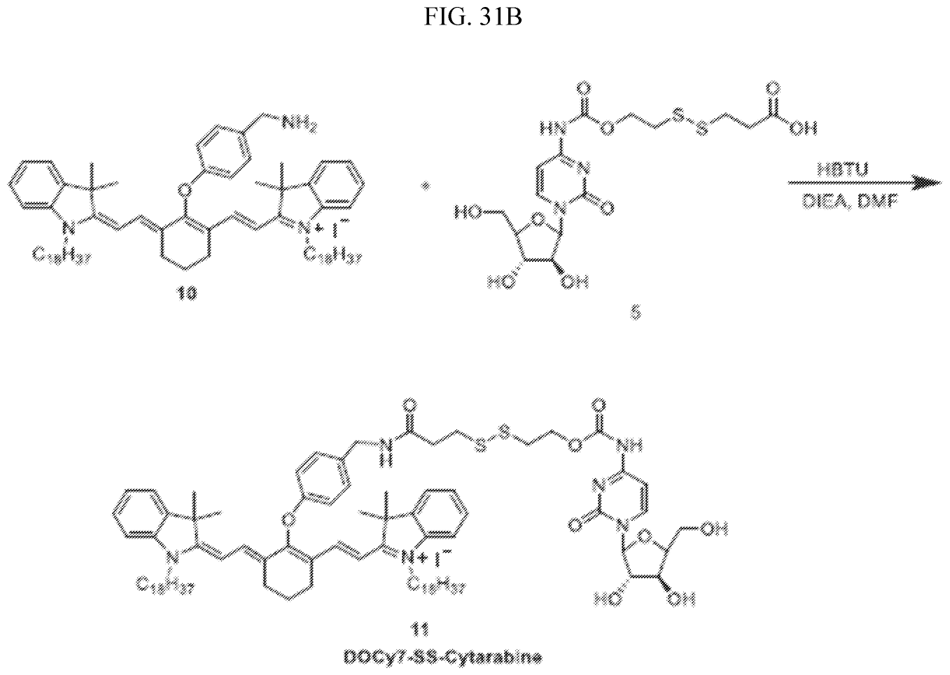

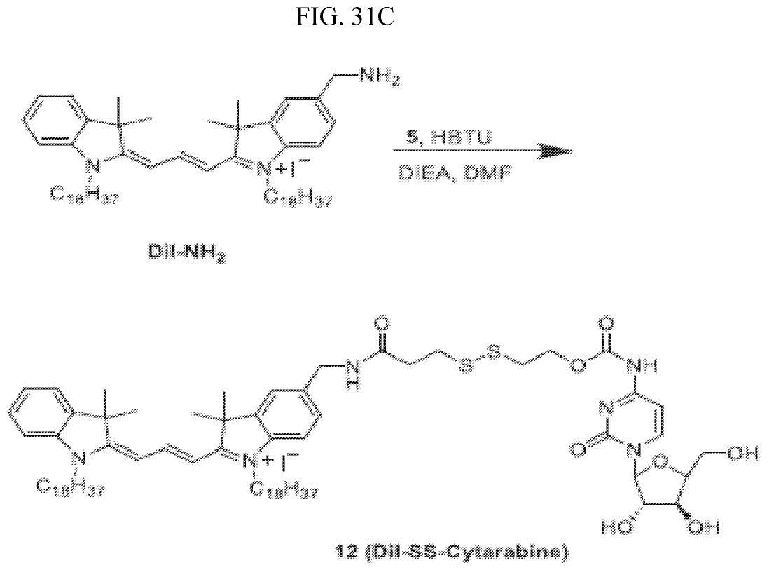

FIGS. 30, 31A-31C depict example of steps of the synthesis of a disulfide linker-cytarabine conjugate.

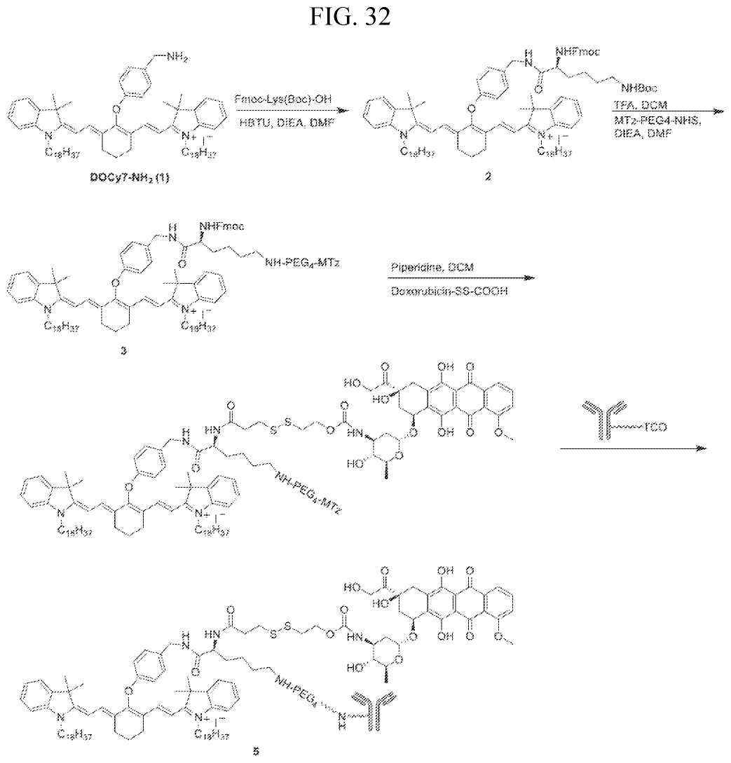

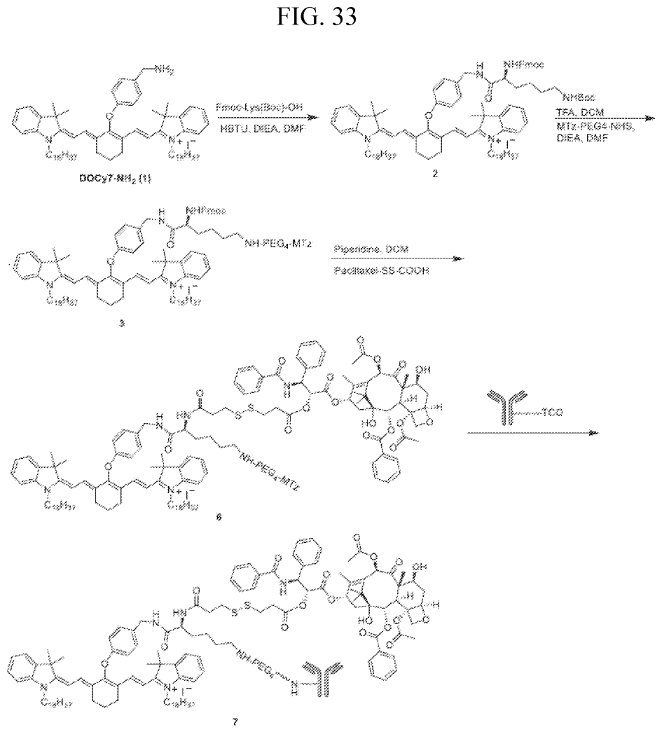

FIGS. 32-33 depict an example of the synthesis of a dual lipid-antibody/drug conjugate.

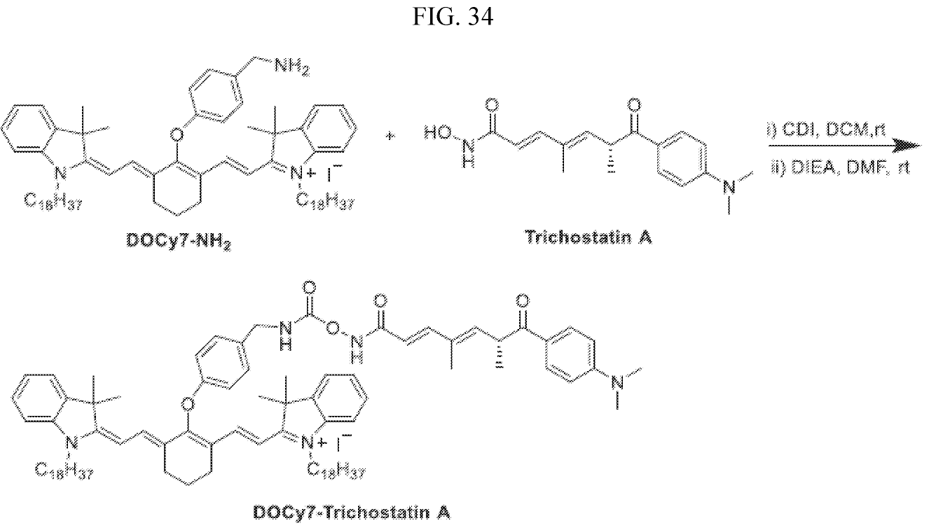

FIG. 34 depicts an example of the synthesis of a lipid-trichostatin A conjugate.

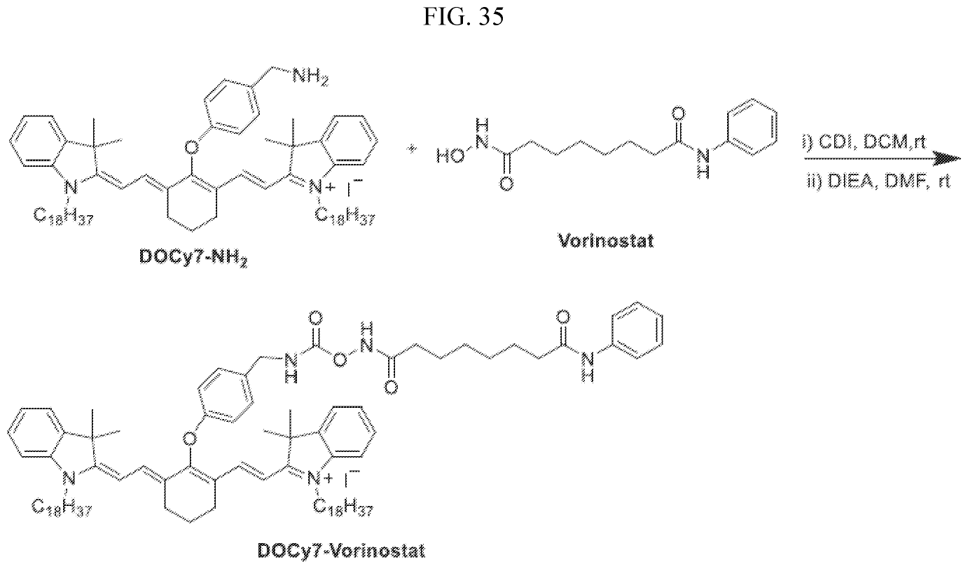

FIG. 35 depicts an example of the synthesis of a lipid-vorinostat conjugate.

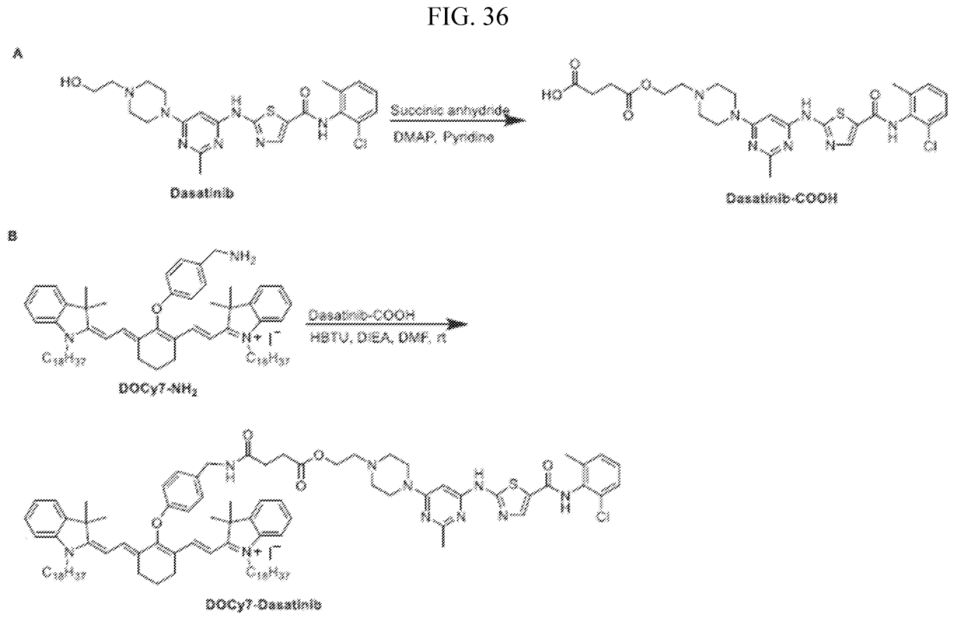

FIG. 36 depicts an example of the synthesis of a lipid-dasatinib conjugate.

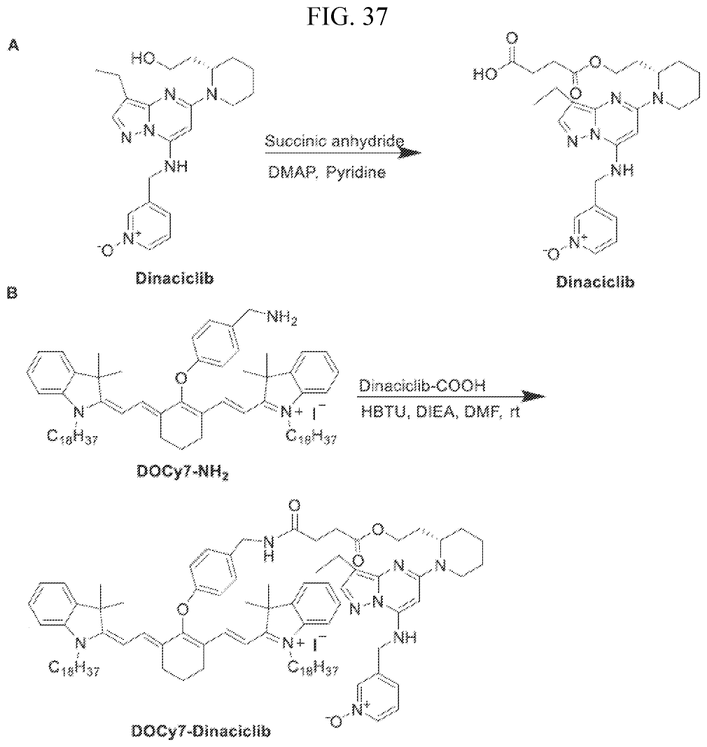

FIG. 37 depicts an example of the synthesis of a lipid-dinaciclib conjugate.

### Example 5: Tuning Organ-Specific Delivery Efficiencies

The present study further discovered that the construct for cargo delivery can be altered to fine tune the delivery efficiencies to individual organs.

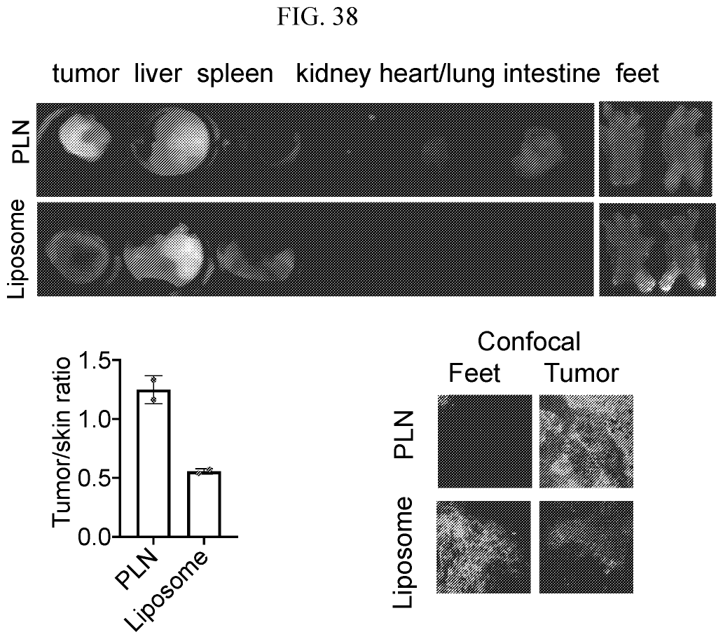

Referring to FIG. 38, PEGylated lipid nanoparticles (PLNs) showed less accumulation than liposomes in plantar skin. This decreased level of skin accumulation is useful when it is desirable to avoid to deliver cargos to the skin. For example, the chemotherapy agent doxorubicin is known to cause damage to the skin (Lotem et al. Arch Dermatol. 2000; 136(12):1475-1480). Using PEGylated lipid nanoparticles to deliver therapeutic agents that can damage skin, such as doxorubicin, would avoid or decrease the undesirable side effects.

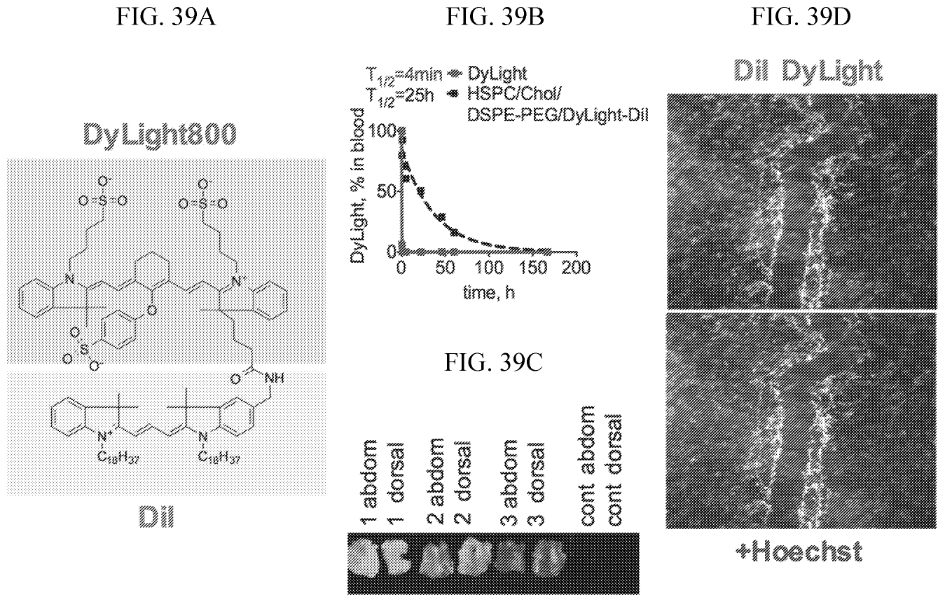

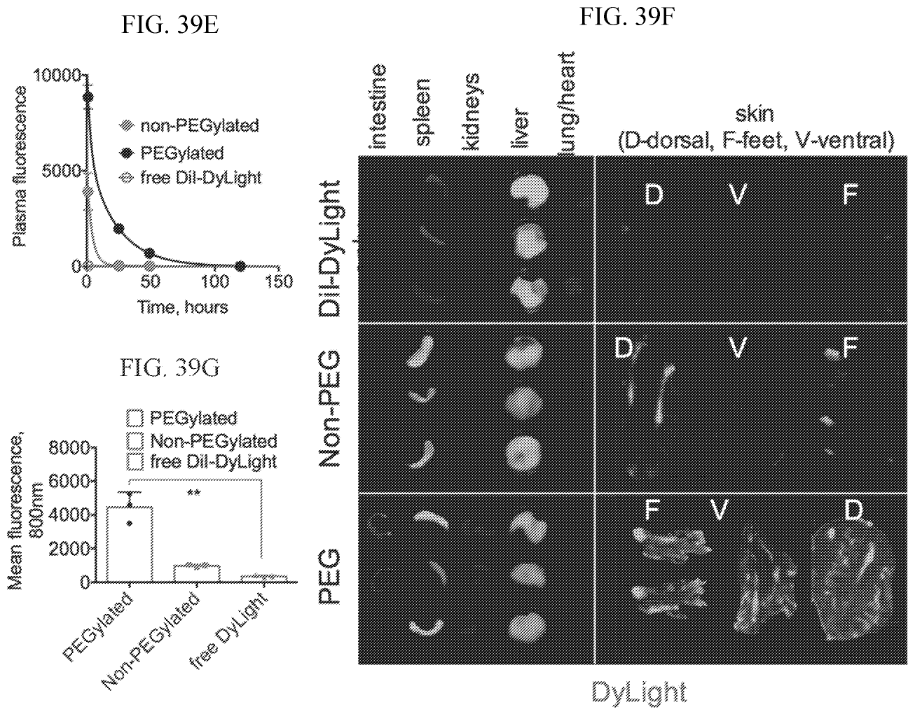

Referring to FIGS. 39A-39G, when ICLs are formulated in liposomes, the construct for cargo delivery showed better accumulation than phospholipids in the skin. For example, referring to FIGS. 39E-39G, the inclusion of the non-limiting ICL DyLight800-DiI in a PEGylated formulation resulted in higher levels of skin accumulation.

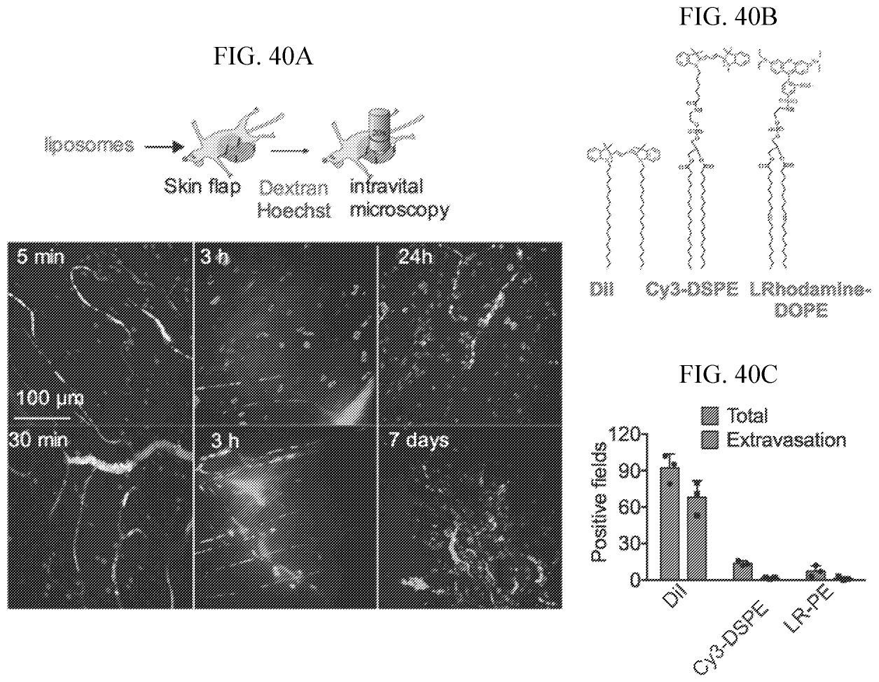

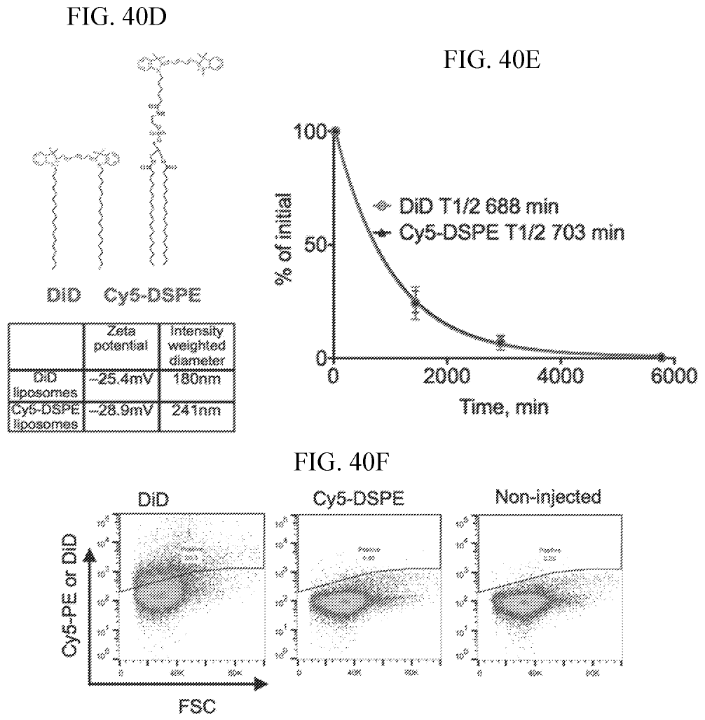

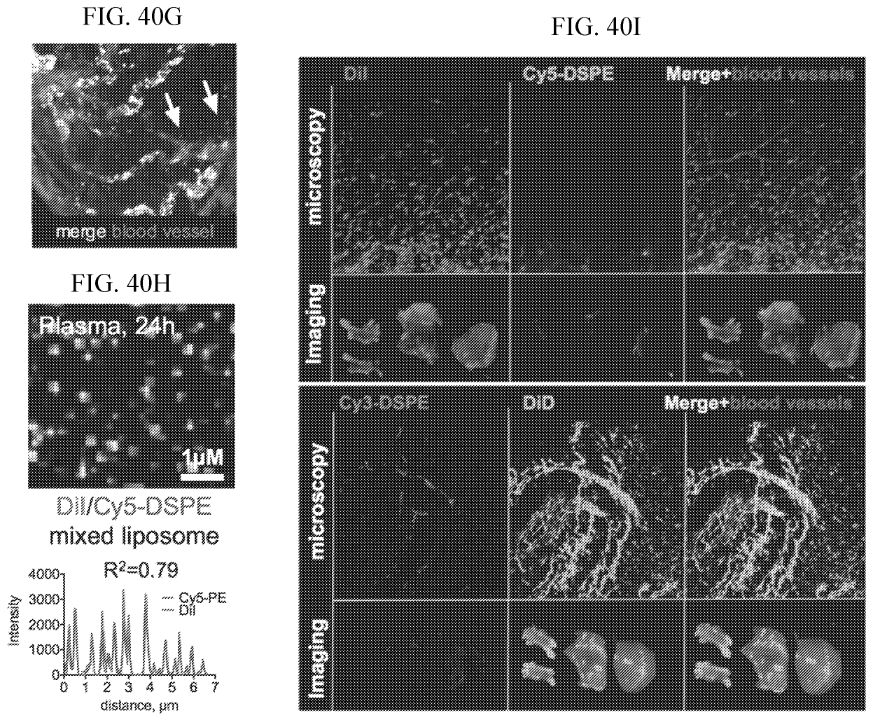

Referring to FIGS. 40A-40I, indocyanine lipids exhibit more efficient skin accumulation than phospholipids.

Since sometimes it is desirable to deliver higher amount of cargo to the skin, being able tune the composition of the construct to increase the skin accumulation is desirable.

### Example 6: Exemplary Construct for Targeting IL13Rα2 Positive Cells

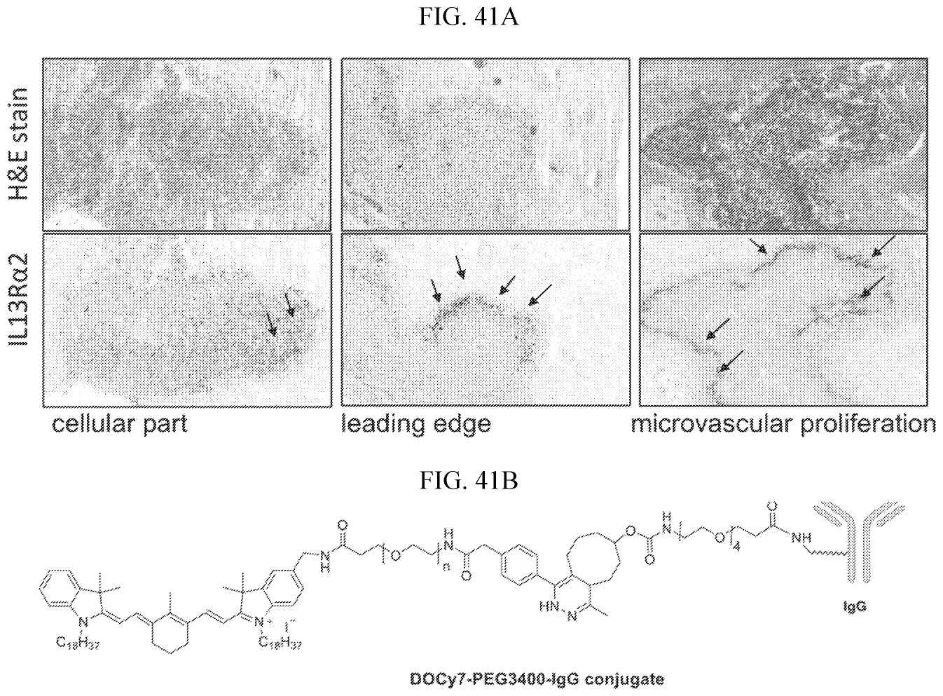

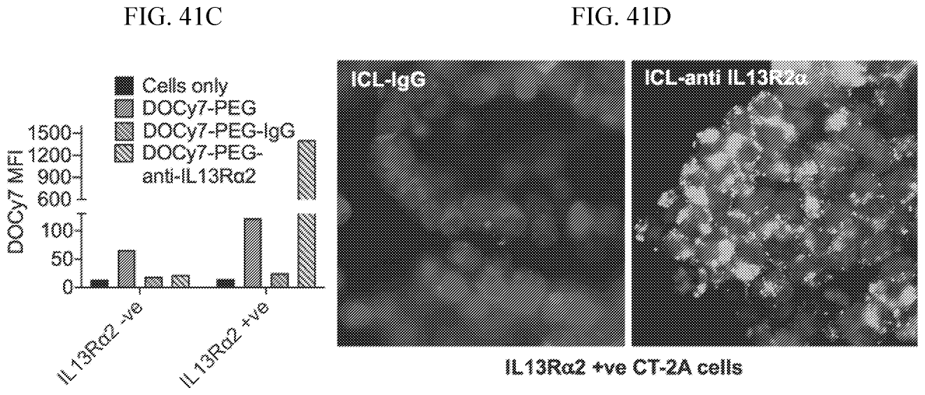

Referring to FIGS. 41A-41D, a non-limiting construct for delivering cargos to IL13Rα2 positive cells were constructed.

Specifically, anti-human IL13Rα2 full IgG was conjugated to the near-infrared ICL DOCy7 via PEG3400 linker (FIG. 41B) using click chemistry (Kolb et al., Angew Chem Int Ed Engl. 2001 Jun. 1; 40(11):2004-2021). Referring to FIGS. 41C-41D, it was demonstrated that the construct targets IL13Rα2 positive CT-2A glioma cells with high efficiency in vitro, but does not significantly target IL13Rα2 negative cells. Interestingly, the conjugation of an antibody improved the selectivity of uptake compared to nonmodified DOCy7-PEG3400.

### Example 7: ICLs have Better Delivery than Other Lipids in Breast Tumors

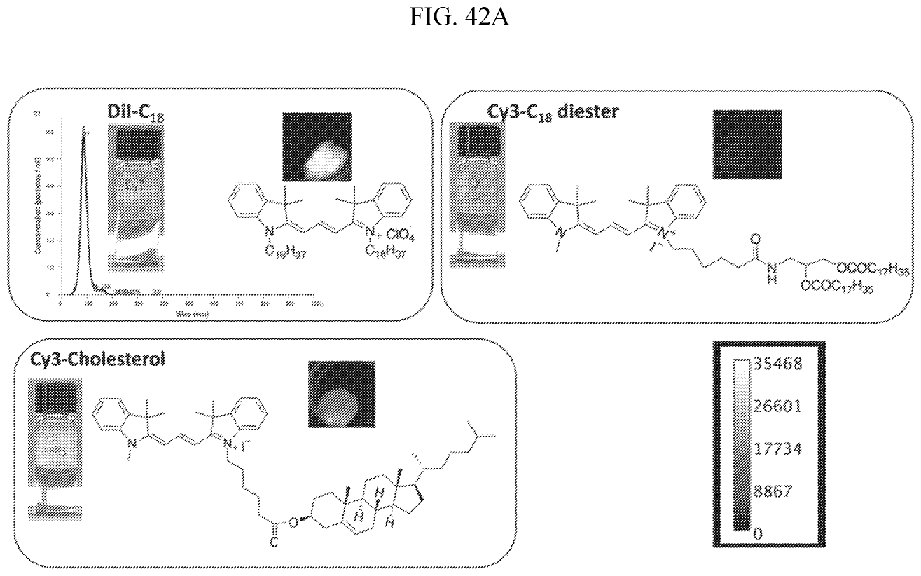

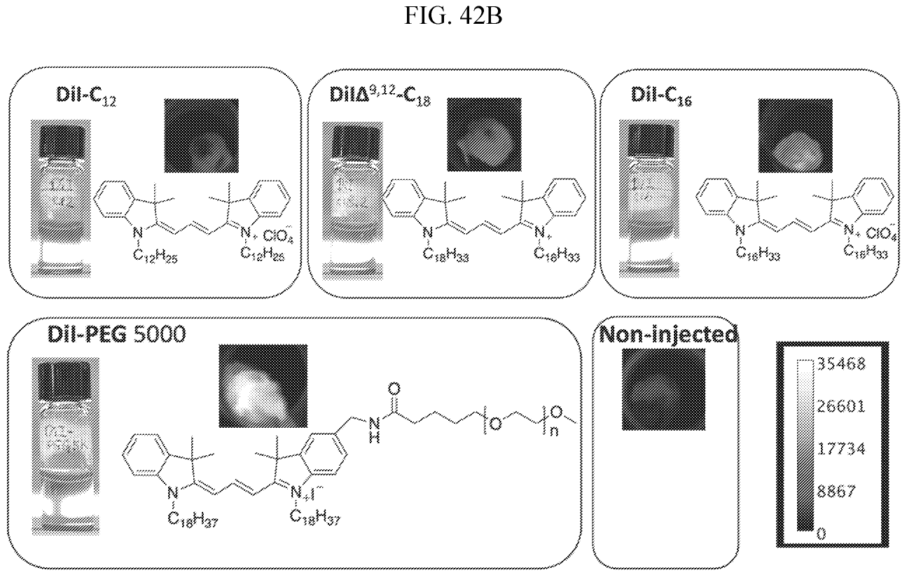

Referring to FIGS. 42A-42B, the present study demonstrated that ICLs have better delivery than other lipids in breast tumors.

Lipids were formulated with DSPE-PEG2000 at 1:2 ratio to form colloidally stable PEGylated lipid nanoparticles, which also include ICLs or other lipids. DiI-PEG5000 was used alone without DSPE-PEG2000.

4T1 mammary carcinoma cells, which are a transplantable tumor cell line, were perfused and excised 48 h post-injection and imaged for Cy3 fluorescence (pseudo-colored inserts). It was shown that the ICLs have better delivery than other lipids in the tumors of the breast cancer model.

### Enumerated Embodiments

The following enumerated embodiments are provided, the numbering of which is not to be construed as designating levels of importance:

Embodiment 1 provides a method of delivering a cargo to a tumor in a subject in need thereof, the method comprising administering a construct to the subject; wherein the construct comprises a lipophilic membrane dye covalently linked through a linker to a cargo; and wherein the lipophilic membrane dye comprises a compound of formula (I) or (II), or a salt, solvate, enantiomer, diastereoisomer, tautomer, or geometric isomer thereof:

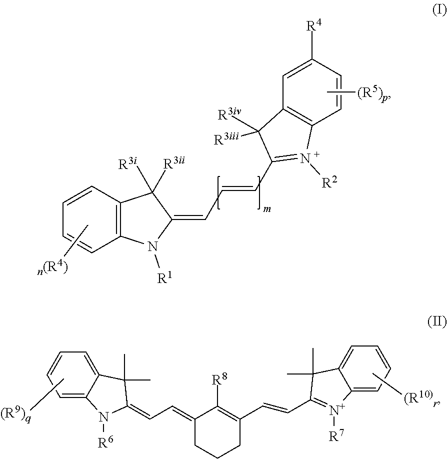

wherein:
R¹, R², R⁶, and R⁷ are each independently C₁₀-C₂₂ alkyl;
R³ⁱ, R³ⁱⁱ, R³ⁱⁱⁱ, and R^{3iv} are each independently selected from the group consisting of H and C₁-C₆ alkyl;
each occurrence of R⁴ is independently selected from the group consisting of C₁-C₆ alkyl, C₃-C₈ cycloalkyl, halo, —NO₂, —CN, —OR^{4a}, —NR^{4a}R^{4a}, —NR^{4a}—C(═O)R^{4a}, —NR^{4a}—SO₂R^{4a}, —C(═O)OR^{4a}, —C(═O)NR^{4a}R^{4a}, —SO₃H, —SR^{4a}, —S(═O)₁₋₂(C₁-C₆ alkyl), and combinations thereof, wherein each occurrence of R^{4a} is independently selected from the group consisting of H and C₁-C₆ alkyl;
each occurrence of R⁵ is independently selected from the group consisting of C₁-C₆ alkyl, C₃-C₈ cycloalkyl, halo, —NO₂, —CN, —OR^{5a}, —NR^{5a}R^{5a}, —NR^{5a}—C(═O)R^{5a}, —NR^{5a}—C(═O)OR^{5a}, —NR^{5a}—SO₂R^{5a}, —C(═O)OR^{5a}, —C(═O)NR^{5a}R^{5a}, —SO₃H, —SR^{5a}, —S(═O)₁₋₂(C₁-C₆ alkyl), and combinations thereof, wherein each occurrence of R^{5a} is independently selected from the group consisting of H and C₁-C₆ alkyl;
R⁸ is selected from the group consisting of C₁-C₆ alkyl, halo, —C(═O)OR^{8a}, —C(═O)NR^{8a}R^{8a}, —O—(C₆-C₁₂ aryl), —NR^{8a}—(C₆-C₁₂ aryl), —O—(C₄-C₁₀ heteroaryl), —NR^{8a}—(C₄-C₁₀ heteroaryl), —NR^{8a}R^{8a}, and combinations thereof, wherein R^{5a} is selected from the group consisting of H and C₁-C₆ alkyl;
each occurrence of R⁹ is independently selected from the group consisting of C₁-C₆ alkyl, C₃-C₈ cycloalkyl, halo, —NO₂, —CN, —OR^{9a}, —NR^{9a}R^{9a}, —NR^{9a}—C(═O)R^{9a}, —NR^{9a}—C(═O)OR^{9a}, —NR^{9a}—SO₂R^{9a}, —C(═O)OR^{9a}, —C(═O)NR^{9a}R^{9a}, —SO₃H, —SR^{9a}, —S(═O)₁₋₂(C₁-C₆ alkyl), and combinations thereof, wherein each occurrence of R^{9a} is independently selected from the group consisting of H and C₁-C₆ alkyl;
each occurrence of R¹⁰ is independently selected from the group consisting of C₁-C₆ alkyl, C₃-C₈ cycloalkyl, halo, —NO₂, —CN, —OR¹⁰a, —NR^{10a}R^{10a}, —NR^{10a}—C(═O)R^{10a}, —NR^{10a}—C(═O)OR^{10a}, —NR^{10a}—SO₂R^{10a}, —C(═O)OR^{10a}, —C(═O)NR^{10a}R^{10a}, —SO₃H, —SR¹⁰a, —S(═O)₁₋₂(C₁-C₆ alkyl), and combinations thereof, wherein each occurrence of R^{10a} is independently selected from the group consisting of H and C₁-C₆ alkyl;
m is 0, 1, 2, 3, 4, 5, or 6; and
n, p, q, and r each independently is 0, 1, or 2.

Embodiment 2 provides the method of Embodiment 1, wherein R³ⁱ, R³ⁱⁱ, R³ⁱⁱⁱ, and R^{3iv} are each methyl.

Embodiment 3 provides the method of any one of Embodiments 1-2, wherein in formula (I), the linker is attached directly to the phenyl ring of at least one indolinyl group, to R⁴, to R⁵, or a combination thereof.

Embodiment 4 provides the method of any one of Embodiments 1-2, wherein in formula (II), the linker is attached directly to the cyclohexene ring, to R⁸, or a combination thereof.

Embodiment 5 provides the method of any one of Embodiments 1-4, wherein the tumor is a brain cancer, head and neck cancer, or breast cancer tumor.

Embodiment 6 provides the method of any one of Embodiments 1-5, wherein the tumor is a glioblastoma multiforme tumor.

Embodiment 7 provides the method of any one of Embodiments 1-6, wherein the cargo is a small molecule, a nucleic acid, a peptide, a protein, or a combination thereof.

Embodiment 8 provides the method of any one of Embodiments 1-7, wherein the cargo is a therapeutic drug, a nucleic acid, a polypeptide, a protein, a chemokine, an aptamer, a nanobody, a minibody, an enzyme, an antibody, a bispecific antibody, a checkpoint inhibitor, a ligand, a biologically active lipid, a transporter substrate, a dye, a chromophore, a fluorophore, a bioluminescent label, a biosensor, a contrast agent, a radioisotope, a hydrophilic polymer, a hydrophilic copolymer, a chemiluminescent label, or a combination thereof.

Embodiment 9 provides the method of any one of Embodiments 1-8, wherein the cargo is a chemotherapeutic agent, an immunostimulant, an antibody, or a combination thereof.

Embodiment 10 provides the method of Embodiment 9, wherein at least one applies: (i) the chemotherapeutic agent is selected from the group consisting of: doxorubicin, auristatin, paclitaxel, cytarabine, trichostatin A, vorinostat, dasatinib, dinaciclib, camptothecin, a STING agonist, and combinations thereof, or (ii) the antibody is an IL13Rα2 scFv.

Embodiment 11 provides the method of any one of Embodiments 1-10, wherein at least one applies: (i) the construct is formulated into a liposome or lipid nanoparticle; or (ii) the cargo is a biologically active lipid selected from a liposome and a lipid nanoparticle.

Embodiment 12 provides the method of any one of Embodiments 1-11, wherein the compound of formula (I) comprises a compound of formula (III), or a salt, solvate, enantiomer, diastereoisomer, tautomer, or geometric isomer thereof:

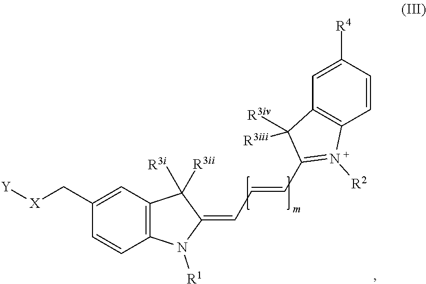

wherein:
R¹ and R² are each independently C₁₀-C₂₂ alkyl;
R³ⁱ, R³ⁱⁱ, R³ⁱⁱⁱ, and R^{3iv} are each independently selected from the group consisting of H and C₁-C₆ alkyl;
R⁴ is H or —SO₃H;
X is selected from —NR—, —NR—C(═O)—, —NR—C(═O)O—, —C(═O)—,

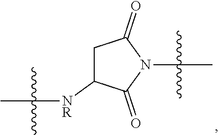

### —NR—S(═O)₂—, —O—, —OC(═O)—, —S—, —S(C═O)—, and —CRR—;

Y comprises the linker conjugated to the cargo;
each R is independently selected from H and C₁-C₆ alkyl; and
m is 0, 1, 2, 3, 4, 5, or 6.

Embodiment 13 provides the method of Embodiment 12, wherein X is —NH—.

Embodiment 14 provides the method of any one of Embodiments 1-11, wherein the compound of formula (II) comprises a compound of formula (IV), or a salt, solvate, enantiomer, diastereoisomer, tautomer, or geometric isomer thereof:

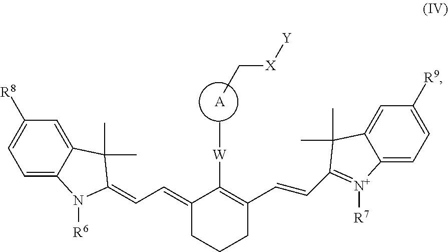

wherein:
R⁶ and R⁷ are each independently C₁₀-C₂₂ alkyl;
R⁸ and R⁹ are each independently H or —SO₃H;
W is —O— or —NR—;
[symbol] is C₆-C₁₂ aryl or C₄-C₁₀ heteroaryl;
X is present and selected from —NR—, —NR—C(═O)—, —NR—C(═O)O—, —C(═O)—,

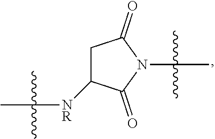

—NR—S(═O)₂—, —O—, —OC(═O)—, —S—, —S(C═O)—, and —CRR—, or X is absent;
Y comprises the linker conjugated to the cargo; and
each R is independently selected from H and C₁-C₆ alkyl.

Embodiment 15 provides the method of Embodiment 14, wherein

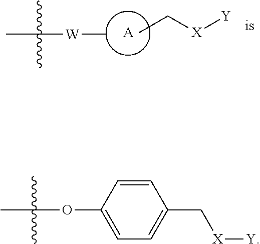

Embodiment 16 provides the method of any one of Embodiments 1-15, wherein the construct is administered to the subject using oral, parenteral, transdermal, transmucosal, intravesical, intrapulmonary, intraduodenal, intragastrical, intrathecal, subcutaneous, intramuscular, intradermal, intra-arterial, intravenous, intrabronchial, inhalation, or topical administration.

Embodiment 17 provides the method of any one of Embodiments 1-16, wherein the construct is configured to have enhanced delivery efficiency or reduced delivery efficiency to the skin.

Embodiment 18 provides a construct comprising a lipophilic membrane dye, wherein the lipophilic membrane dye is covalently linked through a linker to a cargo, and wherein the lipophilic membrane dye comprises a compound of formula (IV), or a salt, solvate, enantiomer, diastereoisomer, tautomer, or geometric isomer thereof:

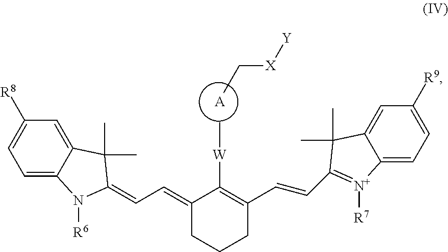

wherein:
R⁶ and R⁷ are each independently C₁₀-C₂₂ alkyl;
R⁸ and R⁹ are each independently H or —SO₃H;
W is —O— or —NR—;
[symbol] is C₆-C₁₂ aryl or C₄-C₁₀ heteroaryl;
X is present and selected from —NR—, —NR—C(═O)—, —NR—C(═O)O—, —C(═O)—,

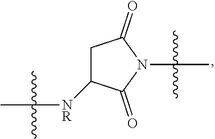

—NR—S(═O)₂—, —O—, —OC(═O)—, —S—, —S(C═O)—, and —CRR—, or X is absent;
Y comprises the linker conjugated to the cargo; and
each R is independently selected from H and C₁-C₆ alkyl.

Embodiment 19 provides the construct of Embodiment 18, wherein [symbol] is C₆ aryl and X is —NH—.

Embodiment 20 provides the construct of any one of Embodiments 18-19, wherein

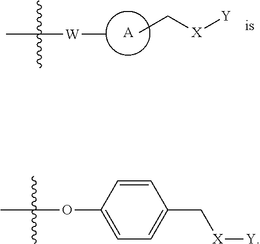

Embodiment 21 provides the construct of any one of Embodiments 18-20, wherein the cargo is a small molecule, a nucleic acid, a peptide, a protein, or a combination thereof.

Embodiment 22 provides the construct of any one of Embodiments 18-21, wherein the cargo is a therapeutic drug, a nucleic acid, a polypeptide, a protein, a chemokine, an aptamer, a nanobody, a minibody, an enzyme, an antibody, a bispecific antibody, a checkpoint inhibitor, a ligand, a biologically active lipid, a transporter substrate, a dye, a chromophore, a fluorophore, a bioluminescent label, a biosensor, a contrast agent, a radioisotope, a hydrophilic polymer, a hydrophilic copolymer, a chemiluminescent label, or a combination thereof.

Embodiment 23 provides the construct of any one of Embodiments 18-22, wherein the cargo is a chemotherapeutic agent, an immunostimulant, an antibody, or a combination thereof.

Embodiment 24 provides the construct of Embodiment 23, wherein at least one applies: (i) the chemotherapeutic agent is selected from the group consisting of doxorubicin, auristatin, paclitaxel, cytarabine, trichostatin A, vorinostat, dasatinib, dinaciclib, camptothecin, a STING agonist, and combinations thereof; or (ii) the antibody is an antibody targeting IL13Rα2.

Embodiment 25 provides the construct of Embodiment 24, wherein in (ii) the antibody is an IL13Rα2 scFv.

Embodiment 26 provides the construct of any one of Embodiments 18-25, wherein at least one applies: (i) the construct is formulated into a liposome or lipid nanoparticle; or (ii) the cargo is a biologically active lipid selected from a liposome and a lipid nanoparticle.

Embodiment 27 provides the construct of any one of Embodiments 18-26, wherein the construct is configured to have enhanced delivery efficiency or reduced delivery efficiency to the skin.

The disclosures of each and every patent, patent application, and publication cited herein are hereby incorporated herein by reference in their entirety. While this invention has been disclosed with reference to specific embodiments, it is apparent that other embodiments and variations of this invention can be devised by others skilled in the art without departing from the true spirit and scope of the invention. The appended claims are intended to be construed to include all such embodiments and equivalent variations.

1. A method of delivering a cargo to a tumor in a subject in need thereof, the method comprising administering a construct to the subject;
wherein the construct comprises a lipophilic membrane dye covalently linked through a linker to a cargo; and
wherein the lipophilic membrane dye comprises a compound of formula (I) or (II), or a salt, solvate, enantiomer, diastereoisomer, tautomer, or geometric isomer thereof: 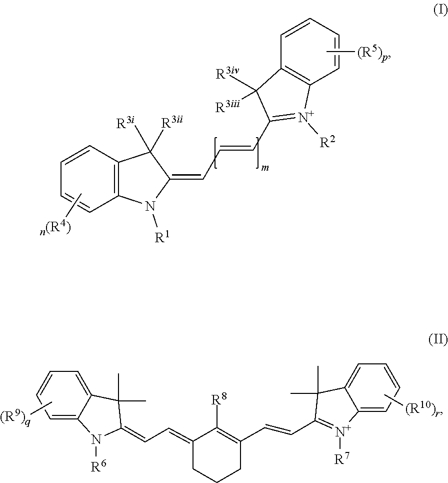
wherein:
R¹, R², R⁶, and R⁷ are each independently C₁₀-C₂₂ alkyl;
R³ⁱ, R³ⁱⁱ, R³ⁱⁱⁱ, and R^{3iv} are each independently selected from the group consisting of H and C₁-C₆ alkyl;
each occurrence of R⁴ is independently selected from the group consisting of C₁-C₆ alkyl, C₃-C₈ cycloalkyl, halogen, —NO₂, —CN, —OR⁴, —NR^{4a}R^{4a}, —NR^{4a}—C(═O)R^{4a}, —NR^{4a}—SO₂R^{4a}, —C(═O)OR^{4a}, —C(═O)NR^{4a}R^{4a}, —SO₃H, —SR^{4a}, —S(═O)₁₋₂(C₁-C₆ alkyl), and combinations thereof,
wherein each occurrence of R^{4a} is independently selected from the group consisting of H and C₁-C₆ alkyl;
each occurrence of R⁵ is independently selected from the group consisting of C₁-C₆ alkyl, C₃-C₈ cycloalkyl, halogen, —NO₂, —CN, —OR^{5a}, —NR^{5a}R^{5a}, —NR^{5a}—C(═O)R^{5a}, —NR^{5a}—C(═O)OR^{5a}, —NR^{5a}—SO₂R^{5a}, —C(═O)OR^{5a}, —C(═O)NR^{5a}R^{5a}, —SO₃H, —SR^{5a}, —S(═O)₁₋₂(C₁-C₆ alkyl), and combinations thereof,
wherein each occurrence of R^{1a} is independently selected from the group consisting of H and C₁-C₆ alkyl;
R⁸ is selected from the group consisting of C₁-C₆ alkyl, halogen, —C(═O)OR^{8a}, —C(═O)NR^{8a}R^{8a}, —O—(C₆-C₁₂ aryl), —NR^{8a}—(C₆-C₁₂ aryl), —O—(C₄-C₁₀ heteroaryl), —NR^{8a}—(C₄-C₁₀ heteroaryl), —NR^{8a}R^{8a}, and combinations thereof,
wherein R^{8a} is selected from the group consisting of H and C₁-C₆ alkyl;
each occurrence of R⁹ is independently selected from the group consisting of C₁-C₆ alkyl, C₃-C₈ cycloalkyl, halogen, —NO₂, —CN, —OR^{9a}, —NR^{9a}R^{9a}, —NR^{9a}—C(═O)R^{9a}, —NR^{9a}—C(═O)OR^{9a}, —NR^{9a}—SO₂R^{9a}, —C(═O)OR^{9a}, —C(═O)NR^{9a}R^{9a}, —SO₃H, —SR^{9a}, —S(═O)₁₋₂(C₁-C₆ alkyl), and combinations thereof,
wherein each occurrence of R^{9a} is independently selected from the group consisting of H and C₁-C₆ alkyl;
each occurrence of R¹⁰ is independently selected from the group consisting of C₁-C₆ alkyl, C₃-C₈ cycloalkyl, halogen, —NO₂, —CN, —OR^{10a}, —NR^{10a}R^{10a}, —NR^{10a}—C(═O)R^{10a}, —NR^{10a}—C(═O)OR^{10a}, —NR^{10a}—SO₂R^{10a}, —C(═O)OR^{10a}, —C(═O)NR^{10a}R^{10a}, —SO₃H, —SR^{10a}, —S(═O)₁₋₂(C₁-C₆ alkyl), and combinations thereof,
wherein each occurrence of R^{10a} is independently selected from the group consisting of H and C₁-C₆ alkyl;
m is 0, 1, 2, 3, 4, 5, or 6; and
n, p, q, and r each independently is 0, 1, or 2.

2. The method of claim 1, wherein R³ⁱ, R³ⁱⁱ, R³ⁱⁱ, and R^{3iv} are each methyl.

3. The method of claim 1,
wherein in formula (I), the linker is attached directly to the phenyl ring of at least one indolinyl group, to R⁴, to R⁵, or a combination thereof; or
wherein in formula (II), the linker is attached directly to the cyclohexene ring, to R⁸, or a combination thereof.

4. (canceled)

5. The method of claim 1, wherein the tumor is a brain cancer, head and neck cancer, or breast cancer tumor: optionally wherein the tumor is a glioblastoma multiforme tumor.

6. (canceled)

7. The method of claim 1, wherein the cargo is a small molecule, a nucleic acid, a peptide, a protein, or a combination thereof.

8. The method of claim 1, wherein the cargo is a therapeutic drug, a nucleic acid, a polypeptide, a protein, a chemokine, an aptamer, a nanobody, a minibody, an enzyme, an antibody, a bispecific antibody, a checkpoint inhibitor, a ligand, a biologically active lipid, a transporter substrate, a dye, a chromophore, a fluorophore, a bioluminescent label, a biosensor, a contrast agent, a radioisotope, a hydrophilic polymer, a hydrophilic copolymer, a chemiluminescent label, a chemotherapeutic agent, an immunostimulant, or a combination thereof.

9. (canceled)

10. The method of claim 8, wherein at least one of the following applies:
(i) the chemotherapeutic agent is selected from the group consisting of: doxorubicin, auristatin, paclitaxel, cytarabine, trichostatin A, vorinostat, dasatinib, dinaciclib, camptothecin, a STING agonist, and combinations thereof,
(ii) the antibody is an IL13Rα2 scFv.

11. The method of claim 1, wherein at least one of the following applies:
(i) the construct is formulated into a liposome or lipid nanoparticle;
(ii) the cargo is a biologically active lipid selected from a liposome and a lipid nanoparticle.

12. The method of claim 1, wherein the compound of formula (I) comprises a compound of formula (III), or a salt, solvate, enantiomer, diastereoisomer, tautomer, or geometric isomer thereof: 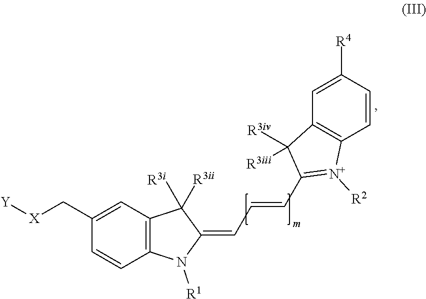
wherein:
R¹ and R² are each independently C₁₀-C₂₂ alkyl;
R³ⁱ, R³ⁱⁱ, R³ⁱⁱⁱ, and R^{3iv} are each independently selected from the group consisting of H and C₁-C₆ alkyl;
R⁴ is H or —SO₃H;
X is selected from —NR—, —NR—C(═O)—, —NR—C(═O)O—, —C(═O)—, 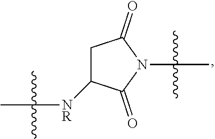
—NR—S(═O)₂—, —O—, —OC(═O)—, —S—, —S(C═O)—, and —CRR—, optionally wherein X is —NH—;
Y comprises the linker conjugated to the cargo;
each R is independently selected from H and C₁-C₆ alkyl; and
m is 0, 1, 2, 3, 4, 5, or 6.

13. (canceled)

14. The method of claim 1, wherein the compound of formula (II) comprises a compound of formula (IV), or a salt, solvate, enantiomer, diastereoisomer, tautomer, or geometric isomer thereof: 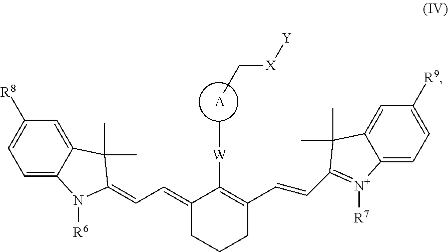
wherein:
R⁶ and R⁷ are each independently C₁₀-C₂₂ alkyl;
R⁸ and R⁹ are each independently H or —SO₃H;
W is —O— or —NR—; [symbol]
is C₆-C₁₂ aryl or C₄-C₁₀ heteroaryl;
X is present and selected from —NR—, —NR—C(═O)—, —NR—C(═O)O—, —C(═O)—, 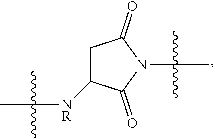
—NR—S(═O)₂—, —O—, —OC(═O)—, —S—, —S(C═O)—, and —CRR—, or X is absent;
Y comprises the linker conjugated to the cargo; and
each R is independently selected from H and C₁-C₆ alkyl.

15. The method of claim 14, wherein is

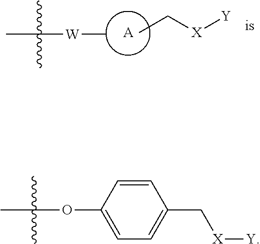

16. The method of claim 1, wherein at least one of the following applies:
(i) the construct is administered to the subject using oral, parenteral, transdermal, transmucosal, intravesical, intrapulmonary, intraduodenal, intragastrical, intrathecal, subcutaneous, intramuscular, intradermal, intra-arterial, intravenous, intrabronchial, inhalation, or topical administration;
(ii) the construct is configured to have enhanced delivery efficiency or reduced delivery efficiency to the skin.

17. (canceled)

18. A construct comprising a lipophilic membrane dye,
wherein the lipophilic membrane dye is covalently linked through a linker to a cargo, and
wherein the lipophilic membrane dye comprises a compound of formula (IV), or a salt, solvate, enantiomer, diastereoisomer, tautomer, or geometric isomer thereof: 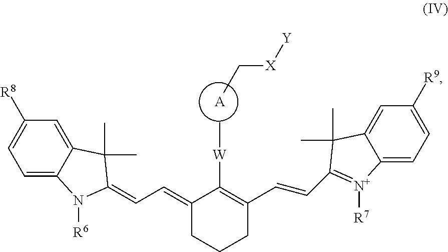
wherein:
R⁶ and R⁷ are each independently C₁₀-C₂₂ alkyl;
R⁸ and R⁹ are each independently H or —SO₃H;
W is —O— or —NR—; [symbol]
is C₆-C₁₂ aryl or C₄-C₁₀ heteroaryl;
X is present and selected from —NR—, —NR—C(═O)—, —NR—C(═O)O—, —C(═O)—, 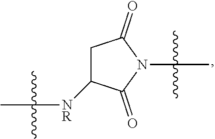
—NR—S(═O)₂—, —O—, —OC(═O)—, —S—, —S(C═O)—, and —CRR—, or X is absent;
Y comprises the linker conjugated to the cargo; and
each R is independently selected from H and C₁-C₆ alkyl.

19. The construct of claim 18, wherein at least one of the following applies:
(i) [symbol] is C₆ aryl and X is —NH—;
(ii)

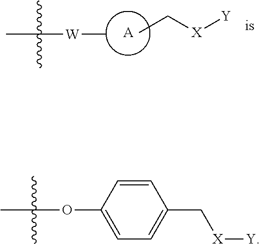

20. (canceled)

21. The construct of claim 18, wherein the cargo is a small molecule, a nucleic acid, a peptide, a protein, or a combination thereof.

22. The method of claim 18, wherein the cargo is a therapeutic drug, a nucleic acid, a polypeptide, a protein, a chemokine, an aptamer, a nanobody, a minibody, an enzyme, an antibody, a bispecific antibody, a checkpoint inhibitor, a ligand, a biologically active lipid, a transporter substrate, a dye, a chromophore, a fluorophore, a bioluminescent label, a biosensor, a contrast agent, a radioisotope, a hydrophilic polymer, a hydrophilic copolymer, a chemiluminescent label, a chemotherapeutic agent, an immunostimulant, or a combination thereof.

23. (canceled)

24. The construct of claim 22, wherein at least one applies:
(i) the chemotherapeutic agent is selected from the group consisting of doxorubicin, auristatin, paclitaxel, cytarabine, trichostatin A, vorinostat, dasatinib, dinaciclib, camptothecin, a STING agonist, and combinations thereof,
(ii) the antibody is an antibody targeting IL13Rα2, optionally wherein the antibody is an IL13Rα2 scFv.

25. (canceled)

26. The construct of claim 18, wherein at least one applies:
(i) the construct is formulated into a liposome or lipid nanoparticle;
(ii) the cargo is a biologically active lipid selected from a liposome and a lipid nanoparticle,
(iii) the construct is configured to have enhanced delivery efficiency or reduced delivery efficiency to the skin.

27. (canceled)
